(19) United States
(12) Patent Application Publication
(10) Pub. No.: US 2025/0017213 A1
(43) Pub. Date: Jan. 16, 2025

(54) COMPOSITION FOR MODULATING MICROBIAL GROWTH

(71) Applicant: DSM AUSTRIA GMBH, Getzersdorf (AT)

(72) Inventor: Mathias SPIEGELHOFER, Getzersdorf (AT)

(21) Appl. No.: 18/711,156

(22) PCT Filed: Nov. 22, 2022
(86) PCT No.: PCT/EP2022/082834

§ 371 Date: May 17, 2024

(30) Foreign Application Priority Data
Nov. 22, 2021 (EP) 21209558.2

(51) Int. Cl. A01N 59/26; A01N 37/10; A01P 1/00; A23K 30/00; A23K 30/15; A23L 3/3508; A23L 3/358; A61K 33/42

(52) U.S. Cl. CPC A01N 59/26; A01N 37/10; A23K 30/00; A23K 30/15; A23L 3/3508; A23L 3/358; A61K 33/42; A01P 1/00

(57) ABSTRACT

The present invention relates to a composition for modulating microbial growth which comprises at least one organic acid and/or at least one salt thereof; and hypophosphorous acid and/or at least one salt thereof as well as to an additive, a method for modulating microbial growth, a composition for use as medicament and a use of the composition.

The present invention relates to a composition for modulating microbial growth, an additive, a method for modulating microbial growth, a composition for use as medicament, and a use of the composition.

It is well known in the field of microbiology that the growth behavior of microorganisms depends on environmental conditions. Certain conditions may favor microbial growth while other conditions may reduce growth. Notably, certain environmental conditions may result in a growth reduction or even growth stagnation of some microorganisms, whereas other microorganisms may not be affected at all or may even show increased growth upon exposure to the same conditions.

In practice, environmental conditions are purposefully adapted by humans to either favor or disfavor microbial growth. For instance, efficient growth of selected suitable microorganisms such as bacteria or yeasts or fungi is desired in the milk processing industry e.g. to produce yogurt or cheese. Further examples where a controlled increase of microbial growth is favored include fermentation processes such as beer brewing or wine making.

On the other hand, attempts are made to avoid or at least delay spoilage of nutritional products such as food, feedstock or feed due to uncontrolled growth of undesired microorganisms. In this regard, temperature conditions may be chosen to disfavor microbial growth. Typically, products subjected to a risk of microbial spoilage may be kept frozen or at least may be stored at low temperatures, e.g. below 10° C. Further options to modulate or control microbial growth include the removal of water e.g. by lyophilization, smoking, salting, curing, sugaring or pickling. In some preservation processes, an acidic pH environment is established to disfavor the growth of unfavorable and potentially pathogenic microorganisms.

In this regard, acidifier products including organic acids may be used to maintain food and feed hygiene and prevent spoilage by microorganisms such as bacteria or molds. Organic acids e.g. formic acid, propionic acid or acetic acid as described in EP 2 642 874 may be used to control microbial growth in foodstuff, food or feed with the aim to minimize the risk for foodborne diseases. To this end, acidifier products may be added pre-and/or post-production. Additionally, acidifier products may be used to achieve a reduced growth of potentially pathogenic microorganisms in the gastrointestinal tract, but also to improve feed conversion rate and weight gain upon ingestion.

Notwithstanding the above, there is a constant need to improve said acidifier products, e.g. by improving the efficiency of the employed acids.

In view of the prior art as outlined above, it is an objective of the present invention to provide efficient means and methods to modulate microbial growth.

This objective is achieved by providing a composition for modulating microbial growth, wherein the composition comprises at least one organic acid and/or at least one salt thereof, and hypophosphorous acid and/or at least one salt thereof. It was unexpectedly found that such a composition was capable of modulating microbial growth, in particular of reducing or inhibiting the growth of potential pathogenic microorganisms. Typically, such potential pathogenic microorganisms belong to either of the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum. Especially strains of E. coli, Salmonella, Shigella, Clostridium, Staphylococcus are often causing pathogenic symptoms. Remarkably, it was found that the degree of inhibition achievable by organic acid(s) or salt(s) thereof alone could be increased beyond a merely additive effect by the addition of hypophosphorous acid or salt(s) thereof. By modulating the microbial growth of certain microorganisms, it is possible to modulate the composition of entire microbial communities (so-called microbiomes), such as the microbiome present in foodstuff, fodder, feed, food, silage, wet distillers grain with solubles, food matrix, feed matrix, or the gut microbiome of an individual, which represent a microbial community present in the gastrointestinal tract of said individual. Merely for clarification, a food/feed matrix is described by the presence of typical feed/food components (e.g. crop, hay, straw, food/feed protein sources such as soy protein, starch and/or other carbohydrates etc.) either prior to or during ingestion by a human or animal, but in any case prior to excretion of said food/feed components.

Such a composition according to the invention may be provided in a form wherein one or more or all of the components is/are provided in solid form (e.g. salt, powder, granulate, pellet etc.) or in liquid form (e.g. aqueous, gel, viscous). It is also considered that the composition may be provided in a manner, wherein one component (e.g. one or more organic acid(s) is provided in liquid form and a second component is provided in solid form (e.g. hypophosphite), and the composition is formed by combining the two components.

In an embodiment, the hypophosphorous acid and/or the at least one salt thereof comprised in the composition of the invention is selected from the group consisting of sodium hypophosphite, magnesium hypophosphite, manganese hypophosphite, potassium hypophosphite, aluminum hypophosphite, calcium hypophosphite, ammonium hypophosphite, ferric hypophosphite, preferably sodium hypophosphite, magnesium hypophosphite, manganese hypophosphite or potassium hypophosphite, more preferably sodium hypophosphite. The use of such hypophosphite compounds was found by the inventors to be especially convenient to handle in the process of preparing the composition. In particular, when this component of the composition is provided in the form of a hypophosphite salt as listed above, mixing and dosing with further component(s) of the composition could be achieved conveniently. It was further found that when the hypophosphorous acid and/or the at least one salt thereof is provided in the form of a hypophosphite salt as listed above, the composition is particularly effective since only surprisingly low amounts of hypophosphite salt are necessary.

In a particular embodiment, the at least one organic acid and/or the at least one salt thereof comprised in the composition referred to herein is selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and keto carboxylic acids, and/or at least one salt thereof. Merely for clarification, examples of short monocarboxylic acids having between 1 and 6 carbon atom(s) are formic acid, acetic acid, propionic acid, butyric acid, isobutyric acid, 3-methylbutyric acid, 2-methylbutyric acid, 2-ethylbutyric acid, valeric acid, hexanoic acid. Examples of saturated dicarboxylic acids are adipic acid, succinic acid. An example of an unsaturated dicarboxylic acid is fumaric acid. Examples of unsaturated carboxylic acids are sorbic acid or oleic acid. Examples of saturated carboxylic acids are stearic acid, octanoic acid (also referred to as caprylic acid), decanoic acid (also referred to as capric acid), dodecanoic acid (also referred to as lauric acid). Examples of hydroxylcarboxylic acids are lactic acid, malic acid (D-, or L-, or D/L-malic acid), citric acid, tartaric acid. Examples of aromatic carboxylic acids are benzoic acid, cinnamic acid. An example of keto carboxylic acid is pyruvic acid. By selecting the at least one organic acid and/or the at least one salt thereof from the group of acids listed above, a particularly well-balanced modulation of microbial growth can be achieved. In particular, growth inhibition of potential pathogens can be achieved, while the growth of non-pathogenic or even probiotic microorganisms is less affected or even not at all. In a further embodiment, the at least one organic acid and/or the at least one salt thereof is selected from the group consisting of formic acid, acetic acid, propionic acid, butyric acid, isobutyric acid, 3-methyl-butyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid, gallic acid, or at least one salt thereof (e.g. ammonium formate, potassium diformate, sodium diacetate, calcium acetate, ammonium propionate, sodium propionate, calcium propionate, calcium lactate, potassium sorbate, sodium formate, calcium formate, sodium butyrate, sodium benzoate, sodium sorbate, potassium citrate, sodium citrate, calcium citrate); preferably from formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, or at least one salt thereof; more preferably from acetic acid, benzoic acid, butyric acid, citric acid, formic acid, fumaric acid, lactic acid, octanoic acid, propionic acid, pyruvic acid, sorbic acid, succinic acid, valeric acid, or at least one salt thereof. When the at least one organic acid and/or the at least one salt thereof comprised in the composition referred to herein is/are selected from the group of acids described above, the composition was found to inhibit the growth of pathogenic microbials particularly efficiently.

When aiming to further optimize the efficiency of the composition according to the invention, said composition may be provided in a particular manner, wherein the molar ratio of the at least one organic acid and/or the at least one salt thereof to the hypophosphorous acid and/or the at least one salt thereof is from 0.1:200 to 500:0.1, preferably from 0.16:100 to 400:0.2 (e.g. from 0.31:100 to 100:0.2). For instance, the at least one organic acid and/or the at least one salt thereof may be provided at any concentration from 0.16 mM to 400 mM, in combination with the hypophosphorous acid and/or at least one salt thereof at any concentration from 0.2 mM to 100 mM. Surprisingly, the composition according to the invention was found to allow modulation of microbial growth throughout the entire large range of a molar ratio of the at least one organic acid and/or the at least one salt thereof to the hypophosphorous acid and/or the at least one salt thereof from 0.1:200 to 500:0.1

In further embodiments, compositions according to the invention may comprise more than one organic acid and/or the at least one salt thereof in combination with the hypophosphorous acid and/or at least one salt thereof. For example, such a composition may comprise two, three, four or even more organic acids in combination with hypophosphorous acid and/or at least one salt thereof. In specific embodiments, the composition comprises at least three organic acids, especially formic acid, acetic acid, propionic acid, and a salt of hypophosphorous acid selected from the group consisting of sodium hypophosphite, manganese hypophosphite, magnesium hypophosphite and potassium hypophosphite, preferably sodium hypophosphite or manganese hypophosphite, more preferably sodium hypophosphite. In further specific embodiments, the composition comprises at least four organic acids, especially formic acid, acetic acid, propionic acid, benzoic acid, and a salt of hypophosphorous acid selected from the group consisting of sodium hypophosphite, manganese hypophosphite, magnesium hypophosphite and potassium hypophosphite, preferably sodium hypophosphite or manganese hypophosphite, more preferably sodium hypophosphite. Such specific compositions of the invention were found to allow excellent targeting (i.e. reduction) of the growth of undesired microorganisms (namely potentially pathogenic microorganisms), while the growth of desirable microrganisms (namely probiotic microorganisms) was not or at least to a lesser extent affected.

It is a further objective of the invention to provide the means for modulating microbial growth (preferably for reducing microbial growth of potentially pathogenic microorganisms) in a form suitable for food, feed, silage etc. This objective was achieved by providing an additive (e.g. foodstuff additive; fodder additive; feed additive; silage additive) comprising a composition according to the invention. Hereby, a format was found for the composition according to the invention and as described above that is suitable for an application e.g. in food, foodstuff, feed, silage etc. Such an additive may comprise further components, such as one or more of the following: Fat-or water-soluble vitamin(s), trace and/or macromineral(s), (proteinogenic) amino acid(s), organic and/or inorganic adsorbant(s), polyunsaturated fatty acid(s), antimicrobial polypeptide(s), pre-and/or probiotic(s), flavoring and/or coloring agent(s), microorganism(s) and/or enzyme(s) to facilitate better nutrient availability and/or to mitigate undesired effect(s) of feed/food contaminant(s). Such an additive may be provided e.g. in liquid and/or in solid form (e.g. salt, powder, granulate, pellet etc., aqueous, gel, viscous; or mixtures thereof wherein one or more components are solid and one or more other component(s) are liquid). In another embodiment, the composition or the additive as referred to herein may be comprised in a feed premix. Hereby, the composition or additive can exert its function instantaneously in the feed premix as well as in the feed to which the feed premix will be added, and continue to modulate microbial growth even upon ingestion of the premix-containing feed.

In a further aspect, the invention relates to a method for modulating microbial growth, wherein the method comprises the steps of a) providing a composition for modulating microbial growth as described herein, and/or an additive as described herein; and b) applying a) to a foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof. It was found that microbial growth could be efficiently modulated or controlled in any of the following compositions: foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof; when a composition or an additive according to the invention is applied. A person having skill in the art is aware of the fact, that microbial growth will be affected from point of application of the composition to the material onwards. Notwithstanding, it is to be understood that the effect exerted by the composition on the microbe(s) may become more pronounced upon further incubation of the mixture of the composition and/or additive with the foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof, under conditions suitable for allowing growth of said microbe(s). It is considered that the method described herein may further relate to a modulation of a microbiome, preferably of the microbiome present in foodstuff, fodder, feed, food, silage, wet distillers grain with solubles, food matrix, feed matrix and/or in the gastrointestinal tract of an individual

In a particular embodiment, the invention relates to the method as described above, wherein the microbial growth is the growth of at least one microbe selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, preferably from Enterobacter, Escherichia, Salmonella, Shigella, Yersinia, Vibrio, Aeromonas, Clostridium, Staphylococcus, Brachyspira, Streptococcus, Moritella, Tenacibaculum more preferably from Escherichia, Salmonella, Shigella, Clostridium, most preferably the microbial growth is the growth of at least one microbe selected from the species of Escherichia coli, Salmonella enterica, Shigella sonnei, Clostridium perfringens. In particular, said microbial growth is reduced. By modulating or controlling, in particular by reducing or suppressing or preventing the microbial growth of one or more of these particular potentially pathogenic microorganisms, the risk for these microbes to cause any disadvantageous effect(s) on the individual (e.g. disease, discomfort, reduction in performance) can be mitigated. In particular, a dysbiosis caused by a high relative abundance of one or more of these potentially pathogenic microorganisms in the microbiome of an individual as a result of uncontrolled growth of one or more of these potentially pathogenic microorganisms can be avoided or reversed.

In a further aspect, the invention relates to a composition for use as a medicament and/or in treatment, amelioration, prophylaxis and/or diagnostics of a disease, such as in veterinary medicine, wherein the composition comprises hypophosphorous acid and/or at least one salt thereof; and optionally at least one organic acid and/or at least one salt thereof. Hereby, growth of microorganisms may be suppressed, e.g. in food or feed or similar matrixes, and at the same time this food or feed does not exert any adverse effect on the gastrointestinal tract of the animal. In a further particular aspect, the invention relates to a composition for use in the treatment, amelioration and/or prophylaxis of dysbiosis, wherein the composition comprises hypophosphorous acid and/or at least one salt thereof; and optionally at least one organic acid and/or at least one salt thereof f. By providing such a composition for use, a means was found which allows effective modulation of an individual's microbiome. In particular a modulation of the microbiome in a manner, wherein the growth of potential pathogens is inhibited or slowed down, while the growth of advantageous, non-pathogenic microorganisms (e.g. probiotic microorganisms) is less affected or even not affected. Hereby, it becomes possible to shift an individual's microbiome towards an increased relative abundance of beneficial (and/or in particular non-pathogenic) microorganisms, and concomittantly achieve a decrease in the relative abundance of unfavorable (i.e. potentially pathogenic) microorganisms.

In a particular embodiment, the invention relates to a composition for use in the treatment, amelioration and/or prophylaxis of dysbiosis according to claim 13, wherein the dysbiosis is caused by atypical concentration of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum, Lactobacillus, Bifidobacterium, Saccharomyces, Bacillus, Pediococcus, Enterococcus, Streptococcus, Propionibacterium, Pseudomonas, Citrobacter, preferably from Enterobacter, Escherichia, Salmonella, Shigella, Yersinia, Vibrio, Aeromonas, Clostridium, Staphylococcus, Brachyspira, Streptococcus, Lactobacillus, Bifidobacterium, Pediococcus, Enterococcus, more preferably from Escherichia, Salmonella, Shigella, Clostridium, Bacillus, Lactobacillus, Enterococcus, most preferably the at least one microorganism is selected from the species of Escherichia coli, Salmonella enterica, Shigella sonnei, Clostridium perfringens, Lactobacillus reuteri, Enterococcus faecium, Bacillus subtilis. A dysbiosis caused by an atypical concentration of such a microorganism or even by atypical concentrations of more than one of these microorganisms, may be treated, mitigated and/or avoided by the use of the composition of the invention.

Merely for the sake of clarity, a dysbiosis as referred to herein may be described by a higher relative concentration or relative abundance of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, in particular selected from the group consisting of Escherichia coli, Salmonella enterica, Shigella sonnei, Clostridium perfringens in the gastrointestinal tract, in particular in the intestinal tract (e.g. stomach, duodenum, jejunum, illeum, cecum, and/or large intestine) of an individual suffering from dysbiosis compared to the relative concentration or relative abundance of the same at least one microorganism of an individual not suffering from dysbiosis. In addition but also alternatively, a dysbiosis as referred to herein may be described by a lower relative concentration or relative abundance of at least one microorganism selected from the genera of Lactobacillus, Bifidobacterium, Saccharomyces, Bacillus, Pediococcus, Enterococcus, Streptococcus, Propionibacterium, Pseudomonas, Citrobacter, in particular selected from the group consisting of Lactobacillus reuteri, Enterococcus faecium, Bacillus subtilis in the gastrointestinal tract, in particular in the intestinal tract (e.g. stomach, duodenum, jejunum, illeum, cecum, and/or large intestine) of an individual suffering from dysbiosis compared to the relative concentration or relative abundance of the same at least one microorganism of an individual not suffering from dysbiosis. Methods for determining presence, concentration, absolute and/or relative abundances of microorganisms are well known in the art, such as metagenome sequence analysis.

In another aspect, the invention relates to a composition for use in the treatment, amelioration and/or prophylaxis of post-weaning diarrhea, wherein the composition comprises hypophosphorous acid and/or at least one salt thereof; and optionally at least one organic acid and/or at least one salt thereof. Post-weaning diarrhea is often caused by strains of Escherichia coli (e.g. Fairbrother et al. 2005. Anim Health Res Rev. 6(1): 17-39), resulting in considerable economic loss and animal malady. In particular, E. coli strains comprising the F4 (K88) adhesin or the F18 adhesin may be responsible for post-weaning diarrhea. Notably, growth of such strains can be reduced in individuals by administering the composition described herein to individuals (e.g. pigs) in risk or in need of post-weaning diarrhea (PWD). Hereby, PWD can be treated, ameliorated and/or prevented.

In a further aspect, the invention relates to a use of hypophosphorous acid and/or at least one salt thereof; and/or a composition according to the invention and as described herein; and/or an additive according to the invention and as described herein for modulating microbial growth. In a particular embodiment, the invention relates to said use, wherein the microbial growth of at least one microbe selected from Escherichia coli, Salmonella enterica, Shigella sonnei and Clostridium perfringens is reduced or suppressed or prevented.

In another embodiment, the invention relates to the use of hypophosphorous acid and/or at least one salt thereof; and/or a composition according to the invention and as described herein; and/or an additive according to the invention and as described herein for modulating microbial growth in a microbiome, thus for modulating the microbiome, preferably the microbiome present in foodstuff, fodder, feed, food, silage, wet distillers grain with solubles, food matrix. feed matrix and/or in the gastrointestinal tract of an individual.

The invention is further characterized by the following items:

Item 1: Composition for modulating (or controlling) microbial growth, wherein the composition comprises i) at least one organic acid and/or at least one salt thereof; and ii) hypophosphorous acid and/or at least one salt thereof.

Item 2: The composition according to item 1, wherein the hypophosphorous acid and/or the at least one salt thereof is/are selected from the group consisting of sodium hypophosphite, magnesium hypophosphite, manganese hypophosphite, potassium hypophosphite, aluminum hypophosphite, calcium hypophosphite, ammonium hypophosphite, ferric hypophosphite, preferably sodium hypophosphite, magnesium hypophosphite, manganese hypophosphite or potassium hypophosphite, more preferably sodium hypophosphite.

Item 3: The composition according to item 1 or 2, wherein the at least one organic acid and/or the at least one salt thereof is/are selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and keto carboxylic acids, and/or at least one salt thereof.

Item 4: The composition according to any one of the preceding items, wherein the at least one organic acid and/or the at least one salt thereof is/are selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid, gallic acid, or at least one salt thereof (e.g. ammonium formate, potassium diformate, sodium diacetate, calcium acetate, ammonium propionate, sodium propionate, calcium propionate, calcium lactate, potassium sorbate, sodium formate, calcium formate, sodium butyrate, sodium benzoate, sodium sorbate, potassium citrate, sodium citrate, calcium citrate); preferably from formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, or at least one salt thereof; more preferably from acetic acid, benzoic acid, butyric acid, citric acid, formic acid, fumaric acid, lactic acid, octanoic acid, propionic acid, pyruvic acid, sorbic acid, succinic acid, valeric acid, or at least one salt thereof.

Item 5: The composition according to any one of the preceding items, wherein the composition comprises at least one organic acid and at least one salt of hypophosphorous acid (i.e. a hypophosphite salt), preferably sodium hypophosphite or manganese hypophosphite, more preferably sodium hypophosphite

Item 6: The composition according to any one of the preceding items, wherein the molar ratio of the at least one organic acid and/or the at least one salt thereof to the hypophosphorous acid and/or at least one salt thereof is from 0.1:200 to 500:0.1, preferably from 0.16:100 to 400:0.2 (e.g. from 0.31:100 to 100:0.2).

Item 7: The composition according to any one of the preceding items, wherein the composition comprises the at least one organic acid and/or the at least one salt thereof at a concentration of 0.3-200 mM (e.g. from 0.3 mM to 200 mM, from 0.3 mM to 100 mM, 0.3 mM, 1.0 mM, 10 mM, 20 mM, 50 mM, 100 mM, 150 mM, 200 mM), preferably at a concentration of 1.0-100 mM (e.g. 1.0 mM, 1.1 mM, 5 mM, 20 mM, 50 mM, 100 mM), more preferably at a concentration of 0.5-50 mM (e.g. 0.6 mM, 0.63 mM, 1 mM, 1.25 mM, 2.5 mM, 5 mM, 6.25 mM, 10 mM, 12 mM, 12.5 mM, 20 mM, 25 mM, 50 mM).

Item 8: The composition according to any one of the preceding items, wherein the composition comprises the hypophosphorous acid and/or at least one salt thereof at a concentration of 0.1-100 mM, preferably at a concentration of 0.2-100 mM (e.g. from 0.2 mM to 100 mM, from 0.2 to 6.25 mM, 0.2 mM, 0.3 mM, 0.39 mM, 0.4 mM, 0.5 mM, 0.6 mM, 0.7 mM 0.78 mM, 0.8 mM, 0.9 mM, 1 mM, 1.0 mM, 1.5 mM, 1.56 mM, 2 mM, 2.0 mM, 2.5 mM, 3 mM, 3.13 mM, 4 mM, 5 mM, 6 mM, 6.25 mM, etc.).

Item 9: The composition according to any one of the preceding items, wherein the composition comprises the at least one organic acid and/or the at least one salt thereof at a concentration of 0.3-200 mM (e.g. from 0.3 mM to 200 mM, from 0.5 mM to 100 mM, 0.3 mM, 1 mM, 1.0 mM, 100 mM, 200 mM), preferably at a concentration of 0.5-100 mM (e.g. from 0.5 mM to 100 mM, from 1.0 mM to 100 mM), more preferably at a concentration of 0.5-50 mM (e.g. 0.6 mM, 0.63 mM, 1 mM, 1.25 mM, 2.5 mM, 5 mM, 6.25 mM, 10 mM, 12 mM, 12.5 mM, 20 mM, 25 mM, 50 mM); and wherein the composition comprises the hypophosphorous acid and/or at least one salt thereof at a concentration of 0.1-100 mM, preferably at a concentration of 0.2-100 mM (e.g. 0.2-6.25 mM, 0.2 mM, 0.3 mM, 0.39 mM, 0.4 mM, 0.5 mM, 0.6 mM, 0.7 mM 0.78 mM, 0.8 mM, 0.9 mM, 1 mM. 1.0 mM, 1.5 mM, 1.56 mM, 2 mM, 2.5 mM, 3 mM, 3.13 mM, 4 mM, 5 mM, 6 mM, 6.25 mM, etc.).

Item 10: The composition according to any one of the preceding items, wherein the composition comprises at least three organic acids, especially formic acid, acetic acid, propionic acid, and a salt of hypophosphorous acid selected from the group consisting of sodium hypophosphite, manganese hypophosphite, magnesium hypophosphite and potassium hypophosphite, preferably sodium hypophosphite or manganese hypophosphite, more preferably sodium hypophosphite.

Item 11: The composition according to item 10, wherein the composition further comprises benzoic acid.

Item 12: The composition according to any one of the preceding items, wherein the composition is capable of reducing the microbial growth of at least one microbe selected from Escherichia coli, Salmonella enterica, Shigella sonnei and Clostridium perfringens; and/or wherein the composition is capable of reducing the microbial growth of at least one microorganism selected from the group consisting of Lactobacillus reuteri, Enterococcus faecium, Bacillus subtilis less than reducing the microbial growth of at least one microbe selected from Escherichia coli, Salmonella enterica, Shigella sonnei and Clostridium perfringens; wherein the microbial growth is reduced in foodstuff, fodder, feed, food, silage, wet distillers grain with solubles, food matrix, feed matrix and/or in the environmental conditions of the gastrointestinal tract of a subject (i.e. an individual, human, animal, patient).

Item 13: The composition according to item 12, wherein the composition is capable of reducing the microbial growth in the environmental conditions of the gastrointestinal tract of a subject (i.e. an individual, human, animal, patient), in particular in the intestinal tract of the subject (e.g. stomach, duodenum, jejunum, illeum, cecum, and/or large intestine).

Item 14: Additive (e.g. foodstuff additive; fodder additive; feed additive; silage additive) comprising a composition for modulating (or controlling) microbial growth according to any one of the preceding items.

Item 15: Method for modulating (or controlling) microbial growth, the method comprising the steps of
a) providing at least one organic acid and/or at least one salt thereof;
b) providing hypophosphorous acid and/or at least one salt thereof;
c) forming a mixture by combining a ) and b);
d) applying the mixture of c) to a foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof; and optionally
e) incubating the product of step d) at a temperature of 5-50° C., preferably 10-40° C., more preferably 15-35° C., for at least 10 min, preferably for at least 30 min, more preferably for at least 1 h (e.g. at least 3, 4, 5, 6, 7, 8, 9, 10, 11, 12, 13, 14, 15, 16, 17, 18, 19, 20 h etc.).

Item 16: Method for modulating (or controlling) microbial growth, the method comprising the steps of
a) providing a composition for modulating (or controlling) microbial growth according to any one of items 1-13, and/or an additive according to item 14;
b) applying a) to a foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof; and optionally
c) incubating the product of step b) at a temperature of 5-50° C., preferably 10-40° C., more preferably 15-35° C., for at least 10 min, preferably for at least 30 min, more preferably for at least 1 h (e.g. at least 3, 4, 5, 6, 7, 8, 9, 10, 11, 12, 13, 14, 15, 16, 17, 18, 19, 20 h etc.).

Item 17: The method according to any one of items 15 or 16, wherein the microbial growth is the growth of at least one microbe selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum, preferably from Enterobacter, Escherichia, Salmonella, Shigella, Yersinia, Vibrio, Aeromonas, Clostridium, Staphylococcus, Brachyspira, Streptococcus, more preferably from Escherichia, Salmonella, Shigella, Clostridium, most preferably the microbial growth is the growth of at least one microbe selected from the species of Escherichia coli, Salmonella enterica, Shigella sonnei, Clostridium perfringens.

Item 18: The method according to any one of items 15-17, wherein the microbial growth is reduced.

Item 19: The method according to any one of items 15-18, wherein the microbial growth is reduced in foodstuff, fodder, feed, food, silage, wet distillers grain with solubles, food matrix, feed matrix; and/or in the environmental conditions of the gastrointestinal tract of a subject (i.e. an individual, human, animal), in particular in the intestinal tract of the subject (e.g. stomach, duodenum, jejunum, illeum, cecum, and/or large intestine).

Item 20: The method according to any one of items 15-19, wherein a modulation (or control) of the microbial growth, in particular a reduction of the microbial growth is determined by
i) providing at least two separate cell suspensions each comprising at least one microbe in a solution suitable for culturing such a microbe;
wherein the at least one microbe is selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum, Lactobacillus, Bifidobacterium, Saccharomyces, Bacillus, Pediococcus, Enterococcus, Streptococcus, Propionibacterium, Pseudomonas, Citrobacter, preferably from Enterobacter, Escherichia, Salmonella, Shigella, Yersinia, Vibrio, Aeromonas, Clostridium, Staphylococcus, Brachyspira, Streptococcus, more preferably from Escherichia, Salmonella, Shigella, Clostridium, most preferably the microbial growth is the growth of at least one microbe selected from the species of Escherichia coli, Salmonella enterica, Shigella sonnei, Clostridium perfringens;
ii) addition of the mixture of c) of item 15, or the composition of any one of items 1-13, and/or the additive of item 14 to a first cell suspension of the at least two separate cell suspensions of i), thus forming a test culture mixture;
iii) cultivating the test culture mixture of ii) as well as an at least second cell suspension (not comprising the mixture of c) of item 15, or the composition of any one of items 1-13, and/or the additive of item 14) of the at least two separate cell suspensions, which at least second cell suspension comprises the same microbe as the test culture mixture as a growth control mixture, under conditions suitable for culturing such a microbe;
iv) determining an increase in biomass in the test culture mixture and in the growth control mixture (e.g. by means of optical density, cell wet weight, cell dry weight etc.) during culturing;
iv) determining a difference in the biomass formed in the test culture mixture and in the growth control mixture, thus identifying the reduction of the microbial growth of said microbe(s) upon treatment with the mixture of c) of item 15, or the composition of any one of items 1-13, and/or the additive of item 14 compared to the microbial growth of the growth control mixture.

Item 21: The method according to any one of the items 15-20, wherein the microbial growth of at least one microorganism selected from the group consisting of Lactobacillus reuteri, Enterococcus faecium, Bacillus subtilis is less reduced than the microbial growth of at least one microbe selected from Escherichia coli, Salmonella enterica, Shigella sonnei and Clostridium perfringens, preferably the microbial growth of Lactobacillus reuteri is less reduced than the microbial growth of Escherichia coli.

Item 22: A composition for use as a medicament and/or in treatment, amelioration, prophylaxis and/or diagnostics of a disease (e.g. in veterinary medicine), wherein the composition comprises i) hypophosphorous acid and/or at least one salt thereof; and optionally ii) at least one organic acid and/or at least one salt thereof.

Item 22b: The composition for use as a medicament and/or in treatment, amelioration, prophylaxis and/or diagnostics of a disease (e.g. in veterinary medicine) according to item 22, wherein the composition comprises i) hypophosphorous acid and/or at least one salt thereof; and ii) at least one organic acid and/or at least one salt thereof

Item 23: A composition for use in the treatment of dysbiosis, wherein the composition comprises i) hypophosphorous acid and/or at least one salt thereof; and optionally ii) at least one organic acid and/or at least one salt thereof.

Item 23b: The composition for use in the treatment of dysbiosis according to item 23, wherein the composition comprises i) hypophosphorous acid and/or at least one salt thereof; and ii) at least one organic acid and/or at least one salt thereof.

Item 24: The composition for use (as a medicament and/or in treatment, amelioration, prophylaxis and/or diagnostics of a disease (e.g. in veterinary medicine) according to item 22 or item 22b; and/or in the treatment of dysbiosis according to item 23 or item 23b, wherein the hypophosphorous acid and/or the at least one salt thereof is/are selected from the group consisting of sodium hypophosphite, magnesium hypophosphite, manganese hypophosphite, potassium hypophosphite, aluminum hypophosphite, calcium hypophosphite, ammonium hypophosphite, ferric hypophosphite, preferably sodium hypophosphite, magnesium hypophosphite, manganese hypophosphite or potassium hypophosphite, more preferably sodium hypophosphite.

Item 25: The composition for use according to any one of items 22b or 23b, wherein the hypophosphorous acid and/or the at least one salt thereof is/are selected from the group consisting of sodium hypophosphite, magnesium hypophosphite, manganese hypophosphite, potassium hypophosphite, aluminum hypophosphite, calcium hypophosphite, ammonium hypophosphite, ferric hypophosphite, preferably sodium hypophosphite, magnesium hypophosphite, manganese hypophosphite or potassium hypophosphite, more preferably sodium hypophosphite; and wherein the at least one organic acid and/or the at least one salt thereof is/are selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and keto carboxylic acids, and/or at least one salt thereof.

Item 26: The composition for use according to any one of items 22b, 23b, 25, wherein the at least one organic acid and/or the at least one salt thereof is/are selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid, gallic acid, or at least one salt thereof (e.g. ammonium formate, potassium diformate, sodium diacetate, calcium acetate, ammonium propionate, sodium propionate, calcium propionate, calcium lactate, potassium sorbate, sodium formate, calcium formate, sodium butyrate, sodium benzoate, sodium sorbate, potassium citrate, sodium citrate, calcium citrate); preferably from formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, or at least one salt thereof; more preferably from acetic acid, benzoic acid, butyric acid, citric acid, formic acid, fumaric acid, lactic acid, octanoic acid, propionic acid, pyruvic acid, sorbic acid, succinic acid, valeric acid, or at least one salt thereof.

Item 27: The composition for use according to any one of items 22-26, wherein the composition comprises at least one organic acid and at least one salt of hypophosphorous acid (i.e. a hypophosphite salt), preferably sodium hypophosphite or manganese hypophosphite, more preferably sodium hypophosphite.

Item 28: The composition for use according to any one of items 22-27, wherein the molar ratio of the at least one organic acid and/or the at least one salt thereof to the hypophosphorous acid and/or at least one salt thereof is from 0.1:200 to 500:0.1, preferably from 0.16:100 to 400:0.2 (e.g. from 0.31:100 to 100:0.2).

Item 29: The composition for use according to any one of items 22-28, wherein the composition comprises the at least one organic acid and/or the at least one salt thereof at a concentration of at least 0.3 mM, such as at a concentration of 0.3-200 mM, preferably at a concentration of 0.5-100 mM, more preferably at a concentration of 0.5-50 mM (e.g. 0.6 mM, 0.63 mM, 1 mM, 1.25 mM, 2.5 mM, 5 mM, 6.25 mM, 10 mM, 12 mM, 12.5 mM, 20 mM, 25 mM, 50 mM).

Item 30: The composition for use according to any one of items 22-29, wherein the composition comprises the hypophosphorous acid and/or at least one salt thereof at a concentration of at least 0.1 mM, such as at a concentration of 0.1-100 mM, preferably at a concentration of 0.2-100 mM (e.g. 0.2-6.25 mM, 0.2 mM, 0.3 mM, 0.39 mM,0.4 mM, 0.5 mM, 0.6 mM, 0.7 mM 0.78 mM, 0.8 mM, 0.9 mM, 1 mM, 1.5 mM, 1.56 mM, 2 mM, 2.5 mM, 3 mM, 3.13 mM, 4 mM, 5 mM, 6 mM, 6.25 mM, etc.).

Item 31: The composition for use according to any one of items 22-30, wherein the composition comprises the at least one organic acid and/or the at least one salt thereof at a concentration of at least 0.3 mM, such as 0.3-200 mM, preferably at a concentration of 0.5-100 mM, more preferably at a concentration of 0.5-50 mM (e.g. 0.6 mM, 0.63 mM, 1 mM, 1.25 mM, 2.5 mM, 5 mM, 6.25 mM, 10 mM, 12 mM, 12.5 mM, 20 mM, 25 mM, 50 mM); and wherein the composition comprises the hypophosphorous acid and/or at least one salt thereof at a concentration of at least 0.1 mM, such as 0.1-100 mM, preferably at a concentration of 0.2-100 mM (e.g. 0.2-6.25 mM, 0.2 mM, 0.3 mM, 0.39 mM, 0.4 mM, 0.5 mM, 0.6 mM, 0.7 mM 0.78 mM, 0.8 mM, 0.9 mM, 1 mM, 1.5 mM, 1.56 mM, 2 mM, 2.5 mM, 3 mM, 3.13 mM, 4 mM, 5 mM, 6 mM, 6.25 mM, etc.)

Item 32: The composition for use according to any one of items 22-31, wherein the composition comprises the at least one organic acid and/or the at least one salt thereof at a concentration of 2-20 mM; and wherein the composition comprises the hypophosphorous acid and/or at least one salt thereof at a concentration of 0.2-50 mM.

Item 33: The composition for use according to any one of items 22-32, wherein the composition comprises at least three organic acids, especially formic acid, acetic acid, propionic acid, and a salt of hypophosphorous acid selected from the group consisting of sodium hypophosphite, manganese hypophosphite, magnesium hypophosphite and potassium hypophosphite, preferably sodium hypophosphite or manganese hypophosphite, more preferably sodium hypophosphite.

Item 34: The composition for use according to any one of items 22-33, wherein the composition further comprises benzoic acid.

Item 35: The composition for use according to any one of items 22-34, wherein the composition is capable of reducing the microbial growth of at least one microbe selected from Escherichia coli, Salmonella enterica, Shigella sonnei and Clostridium perfringens; and/or wherein the composition is capable of reducing the microbial growth of at least one microorganism selected from the group consisting of Lactobacillus reuteri, Enterococcus faecium, Bacillus subtilis less than reducing the microbial growth of at least one microbe selected from Escherichia coli, Salmonella enterica, Shigella sonnei and Clostridium perfringens.

Item 36: The composition for use according to any one of items 22-35, wherein the composition is capable of reducing the microbial growth in the gastrointestinal tract of a subject (e.g. an individual, a human, an animal, a patient), in particular in the intestinal tract of the subject (e.g. stomach, duodenum, jejunum, illeum, cecum, and/or large intestine).

Item 37: The composition for use according to any one of items 23-36, wherein the dysbiosis is caused by atypical (or aberrant) concentration of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, Lactobacillus, Bifidobacterium, Saccharomyces, Bacillus, Pediococcus, Enterococcus, Streptococcus, Propionibacterium, Pseudomonas, Citrobacter, preferably from Enterobacter, Escherichia, Salmonella, Shigella, Yersinia, Vibrio, Aeromonas, Clostridium, Staphylococcus, Brachyspira, Streptococcus, Lactobacillus, Bifidobacterium, Pediococcus, Enterococcus, more preferably from Escherichia, Salmonella, Shigella, Clostridium, Bacillus, Lactobacillus, Enterococcus, most preferably the at least one microorganism is selected from the species of Escherichia coli, Salmonella enterica, Shigella sonnei, Clostridium perfringens, Lactobacillus reuteri, Enterococcus faecium, Bacillus subtilis.

Item 38: The composition for use according to any one of items 23-37, wherein the dysbiosis is caused by atypically (or aberrantly) high concentration of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, in particular selected from the group consisting of Escherichia coli, Salmonella enterica, Shigella sonnei, Clostridium perfringens in the gastrointestinal tract of a subject (e.g. an individual, a human, an animal, a patient); and/or by atypically low concentration of at least one microorganism selected from the genera of Lactobacillus, Bifidobacterium, Saccharomyces, Bacillus, Pediococcus, Enterococcus, Streptococcus, Propionibacterium, Pseudomonas, Citrobacter, in particular selected from the group consisting of Lactobacillus reuteri, Enterococcus faecium, Bacillus subtilis in the gastrointestinal tract of a subject (e.g. an individual, a human, an animal, a patient), in particular in the intestinal tract of the subject (e.g. cecum, small intestine, and/or large intestine).

Item 39: The composition for use according to any one of items 23-38, wherein the relative concentration or abundance of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, in particular selected from the group consisting of Escherichia coli, Salmonella enterica, Shigella sonnei, Clostridium perfringens in the gastrointestinal tract, in particular in the intestinal tract (e.g. stomach, duodenum, jejunum, illeum, cecum, and/or large intestine) of an individual suffering from dysbiosis is higher than the relative concentration or abundance of the same microorganism(s) in the gastrointestinal tract, in particular in the intestinal tract (e.g. stomach, duodenum, jejunum, illeum, cecum, and/or large intestine) of an individual not suffering from dysbiosis; and/or wherein the relative concentration or abundance of at least one microorganism selected from the genera of Lactobacillus, Bifidobacterium, Saccharomyces, Bacillus, Pediococcus, Enterococcus, Streptococcus, Propionibacterium, Pseudomonas, Citrobacter, in particular selected from the group consisting of Lactobacillus reuteri, Enterococcus faecium, Bacillus subtilis in the gastrointestinal tract, in particular in the intestinal tract (e.g. stomach, duodenum, jejunum, illeum, cecum, and/or large intestine) of an individual suffering from dysbiosis is lower than the relative concentration or abundance of the same microorganism(s) in the gastrointestinal tract, in particular in the intestinal tract (e.g. stomach, duodenum, jejunum, illeum, cecum, and/or large intestine) of an individual suffering from dysbiosis of an individual not suffering from dysbiosis.

Item 40: A composition for use in the treatment, amelioration and/or prophylaxis of post-weaning diarrhea in an individual suffering from and/or in risk of post-weaning diarrhea, wherein the composition comprises i) hypophosphorous acid and/or at least one salt thereof; and optionally ii) at least one organic acid and/or at least one salt thereof.

Item 41: The composition for use in the treatment, amelioration and/or prophylaxis of post-weaning diarrhea according to item 40, wherein the post-weaning diarrhea is caused by one or more strain(s) of enterotoxigenic Escherichia coli (ETEC), and/or by one or more E. coli strain(s) comprising F4 (K88) and/or F18 adhesin(s).

Item 42: The composition for use in the treatment, amelioration and/or prophylaxis of post-weaning diarrhea according to any one of items 40 and 41, wherein the individual suffering from and/or in risk of post-weaning diarrhea belongs to the genus Sus, in particular the individual suffering from and/or in risk of post-weaning diarrhea belongs to the species Sus domesticus.

Item 43: The composition for use in the treatment, amelioration and/or prophylaxis of post-weaning diarrhea according to any one of items 40-42, wherein the composition is the composition according to any one of items 2-13 and/or the additive according to item 14.

Item 44: Use of hypophosphorous acid and/or at least one salt thereof; and/or a composition according to any one of items 1-13; and/or an additive according to item 14 for modulating (or controlling) microbial growth, in particular for reducing or inhibiting microbial growth.

Item 45: The use according to item 44, wherein the microbial growth of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, in particular selected from the group consisting of Escherichia coli, Salmonella enterica, Shigella sonnei, Clostridium perfringens is reduced; and/or wherein microbial growth of at least one microorganism selected from the genera of Lactobacillus, Bifidobacterium, Saccharomyces, Bacillus, Pediococcus, Enterococcus, Streptococcus, Propionibacterium, Pseudomonas, Citrobacter, in particular selected from the group consisting of Lactobacillus reuteri, Enterococcus faecium, Bacillus subtilis is less reduced than the microbial growth of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Moritella, Tenacibaculum, in particular selected from the group consisting of Escherichia coli, Salmonella enterica, Shigella sonnei, Clostridium perfringens.

Item 46: The use according to any one of items 44 or 45, wherein the microbial growth is modulated (or controlled) in the environmental conditions of the gastrointestinal tract, in particular in the environmental conditions of the intestinal tract (e.g. stomach, duodenum, jejunum, illeum, cecum, and/or large intestine) of an individual (e.g. human, animals such as pet, dog, cat, canary, hamster, deer, boar, poultry, swine, ruminant, chicken, cow, sheep, goat, pig, piglet, turkey, aquaculture, fish, shrimp, prawn, crayfish, crab, oyster, mussel, clam, trout, tilapia, salmon, carp, catfish, tuna); and/or wherein the microbial growth is modulated (or controlled) in foodstuff; fodder; feed; silage; wet distillers grain; dried distillers grain with solubles; nutritional supplement; prebiotic; probiotic; intermediate/s thereof; and/or mixture/s thereof.

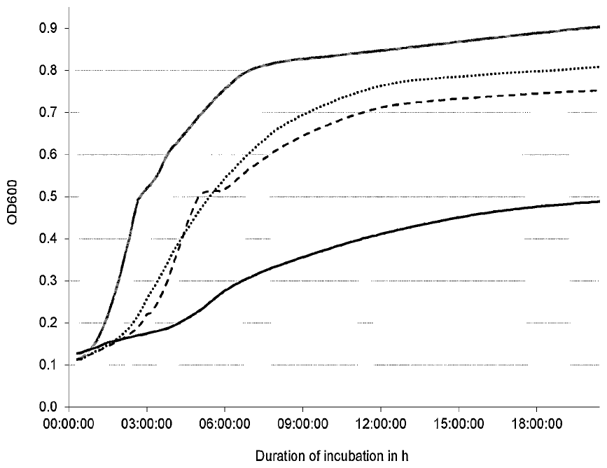

In the following, the present invention is further described by non-limiting figures and examples, wherein FIG. 1 shows the growth of E. coli when treated with either a composition comprising organic acid, or with a composition comprising hypophosphite, or with a composition comprising organic acid and hypophosphite, as well as the growth of E. coli when not treated with either of these compositions. The x-axis shows the absorption at 600 nm (OD600), the y-axis shows the time course of the incubation. The FIGURE shown herein and described below serves merely as illustrative example and is not to be construed as limiting embodiment(s) of the present invention.

### EXAMPLES

The present invention as disclosed herein is not limited to specific embodiments, figures, methodology, examples, protocols etc. described herein but solely defined by the claims. Examples disclosed in the following may only be considered to refer to exemplary embodiments of the underlying invention as defined in the claims.

### Example 1: Modulation of Microbial Growth

To identify compositions capable of modulating microbial growth, compositions comprising either organic acid(s) and/or salt(s) thereof alone; or comprising hypophosphoric acid(s) and/or salt(s) thereof alone; or comprising combinations of i) organic acid(s) and/or salt(s) thereof with ii) hypophosphoric acid(s) and/or salt(s) thereof were tested in growth inhibition tests. A first goal of the performed experiments was to identify compositions capable of reducing microbial growth of typical potential pathogens, such as Escherichia coli, Salmonella enterica enterica, Shigella sonnei or Clostridium perfringens. Based thereon, it was another goal to identify compositions that not only reduce the growth of pathogenic microbes but which compositions at the same time do not affect the growth of desirable, helpful (i.e. typical probiotic) microbes, such as Lactobacillus reuteri, Enterococcus faecium or Bacillus subtilis to the same extent as of the typical potential patogens.

Notably, it was found that compositions comprising organic acid(s) and/or salt(s) thereof and hypophosphorous acid(s) and/or salt(s) thereof were capable of inhibiting the growth of typical pathogenic microorganisms while inhibiting the growth of typical probiotic microorganisms to a lesser extent. For instance, while growth of E. coli was inhibited by more than 95% when treated with a composition comprising 25 mM acetic acid and any one of the concentrations 0.2, 0.39, 0.78, 1.56, 3.13, 6.25, 12.5, 25, 50, or 100 mM of sodium hypophosphite, growth of Lactobacillus reuteri (L. reuteri F 275 (DSMZ 20016)) was not affected (i.e. less than 1% inhibition). Also when either formic acid, or propionic acid, or a combination of formic acid, acetic acid and propionic acid, or a combination of formic acid, acetic acid, propionic acid and benzoic acid was used at combined concentration of 25 mM of acid and either 0.2, 0.39, 0.78, 1.56, 3.13, 6.25, 12.5, 25, 50, or 100 mM of sodium hypophosphite, growth of probiotic bacteria (such as Lactobacillus reuteri, Enterococcus faecium or Bacillus subtilis) was less affected than growth of typical pathogens. For instance, growth of Enterococcus faecium (E. faecium D, serotype 11 (DSMZ 20477)) was only inhibited at most by approximately 2.5% when treated with 25 mM of acetic acid in combination with either 0.2, 0.39, 0.78, 1.56, 3.13, 6.25, 12.5, 25, 50, or 100 mM of sodium hypophosphite. Similarly, growth of Bacillus subtilis (B. subtilis strain Marburg (DSMZ 10)) was found to be inhibited at most by approximately 8.5% when treated with 12.5 mM formic acid in combination with either 0.2, 0.39, 0.78, 1.56, 3.13, 6.25, 12.5, 25, 50, or 100 mM of sodium hypophosphite.

As an example, the growth of E. coli in a typical growth medium (Mueller-Hinton broth) is shown in FIG. 1 when treated with a composition comprising only organic acid, compared to treatment with a composition comprising only hypophosphite salt, compared to treatment with a composition according to the invention (i.e. comprising both, organic acid(s) as well as hypophosphite salt(s), compared to a growth control (i.e. without treatment with a composition comprising either organic salt(s) and/or hypophosphite(s)). In FIG. 1, the solid grey line shows the growth of the E. coli growth control. The dashed black line shows the growth of E. coli when treated with a composition comprising 12.5 mM formic acid. The dotted black line shows the growth of E. coli when treated with a composition comprising 12.5 mM sodium hypophosphite. The solid black line shows the growth of E. coli when treated with a composition comprising both, 12.5 mM formic acid and 12.5 mM sodium hypophosphite. Notably, the microbial growth was found to by synergistically inhibited upon treatment with a combination composition comprising organic acid as well as hypophosphite already after 4 h of incubation at the latest. Exemplary amounts of inhibition of microbial growth throughout the incubation are shown in Table 1 below.

TABLE 1
The amount of inhibition of E. coli growth upon treatment with
either of three compositions (comprising either organic acid,
or hypophosphite salt, or both) is shown, compared to
the microbial growth without such treatment.

| | | % inhibition | % inhibition |
|---|---|---|---|
| incubation | % inhibition | hypophosphite | organic acid + hypophosphite |
| time | organic acid | salt | salt |
| 4 h | 45.5 | 40.5 | 68.9 |
| 5 h | 27.5 | 32.7 | 66.9 |
| 7.5 h | 27.3 | 22.0 | 60.2 |
| 10 h | 19.4 | 13.5 | 54.9 |
| 15 h | 15.6 | 9.6 | 48.1 |
| 20 h | 16.6 | 10.5 | 46.0 |

In contrast to the synergistic inhibition of the microbial growth of a potential pathogen, i.e. E. coli, as shown above, the growth of probiotic bacteria was considerably less affected by treatment with a combination composition comprising both, organic acid as well as hypophosphite salt. Exemplarily, amounts of inhibition of the growth of Lactobacillus reuteri upon treatment with either a composition comprising 12.5 mM formic acid, or with a composition comprising 12.5 mM sodium hypophosphite, or with a composition comprising both 12.5 mM formic acid and 12.5 mM sodium hypophosphite, compared to growth without such treatment are shown in Table 2 below. Notably, treatment with either hypophosphite alone or in combination with organic acid did not affect the growth of the probiotic. Thus, no synergistic effects could be determined with regard to the inhibition of the microbial growth of probiotic bacteria upon treatment with a combination composition comprising organic acid(s) and hypophosphite.

TABLE 2
The amount of inhibition of L. reuteri growth upon treatment with
either of three compositions (comprising either organic acid,
or hypophosphite salt, or both) is shown, compared to
the microbial growth without such treatment.

| | % inhibition | % inhibition | % inhibition |
|---|---|---|---|
| time of | organic | hypophosphite | organic acid + hypophosphite |
| incubation | acid | salt | salt |
| 4 h | 23.4 | 0.6 | 22.9 |
| 5 h | 38.2 | 0.5 | 37.3 |
| 6 h | 39.8 | 0.1 | 39.7 |
| 7 h | 39.7 | 0.3 | 39.6 |
| 10 h | 39.5 | 2.0 | 38.8 |

In a systematic approach, compositions comprising either 1.56, 3.13, 6.25, 12.5, 25, 50 or 100 mM of either acetic acid; butyric acid; citric acid; formic acid; lactic acid; octanoic acid; propionic acid; pyruvic acid; succinic acid; or valeric acid; or comprising acid combinations of formic acid+acetic acid+propionic acid (ratio of 2:1:0.5 of formic acid: acetic acid: propionic acid; e.g. 57 mM formic acid+29 mM acetic acid+14 mM propionic acid for a target concentration of 100 mM acid); or formic acid+acetic acid+propionic acid+benzoic acid (ratio of 2:1: 0.5:1.18 of formic acid: acetic acid: propionic acid: benzoic acid; e.g. 34 mM formic acid+17 mM acetic acid+9 mM propionic acid+20 mM benzoic acid for a target concentration of 80 mM acid), wherein the total acid concentration in these acid combinations added up to either concentration of 1.56, 3.13, 6.25, 12.5, 25, 50 or 100 mM (in case of the combination formic acid+acetic acid+propionic acid) or to either concentration of 1,25, 2,5, 5, 10, 20, 40 or 80 mM (in case of the combination formic acid+acetic acid+propionic acid+benzoic acid) were prepared. Further, compositions comprising either 0.31, 0.63, 1.25, 2.5, 5, 10, 20 mM of benzoic acid; compositions comprising either 6.25, 12.5, 25, 50, 100, 200 or 400 mM of calcium formate; or of sodium benzoate; compositions comprising either 0.39, 0.78, 1.56, 3.13, 6.25, 12,5 or 25 mM of fumaric acid; as well as compositions comprising either 0.16, 0.31, 0.63, 1.25, 2.5, 5, or 10 mM of sorbic acid were prepared. Also, compositions comprising either 0.2, 0.39, 0.78, 1.56, 3.13, 6.25, 12.5, 25, 50, or 100 mM of either sodium hypophosphite; magnesium hypophosphite; or potassium hypophosphite were prepared. Microbial growth was recorded for typical pathogen microbes selected from Escherichia coli (E. coli U5/41 (DSMZ 30083)), Salmonella enterica (S. enterica subsp. enterica serotype typhimurium SL1344 (DSMZ 24522), Shigella sonnei (S. sonnei ATCC 29930, I virulent, WDCM 00127, CECT 4887 (DSMZ 5570), Clostridium perfringens (C. perfringens (CCUG 47895; type A as described in Johansson et al. 2004. Vet Microbiol. 99 (3-4): 251-257)), and for typical probiotic microbes selected from Lactobacillus reuteri (L. reuteri F 275 (DSMZ 20016), Enterococcus faecium (E. faecium D, serotype 11 (DSMZ 20477)), Bacillus subtilis (B. subtilis strain Marburg (DSMZ 10)), upon treatment with compositions as described above, wherein the influence of compositions comprising either organic acid(s) or salt(s) thereof alone on the microbial growth, and the influence of compositions comprising either hypophosphoric acid(s) or salt(s) thereof alone, was compared to the influence of compositions comprising organic acid(s) or salt(s) thereof as well as hypophosphoric acid(s) or salt(s) thereof on the microbial growth.

According to the goals of these experiments, the potential of these compositions either taken alone or in combinations of the organic acid(s) or the salt(s) thereof with sodium hypophosphite, magnesium hypophosphite or potassium hypophosphite to modulate microbial growth was studied. Hereto, compositions comprising acid(s) or salt(s) thereof and/or sodium hypophosphite, magnesium hypophosphite, or potassium hypophosphite as described above were prepared in Mueller-Hinton broth and sterilized by micro-filtration using a 0.22 μm filter. Either of the microorganisms listed above were cultivated in Mueller-Hinton broth (Mueller et al. 1941. Experimental Biology and Medicine 48(1): 330-333) until late exponential phase. Those liquid cultures were used to inoculate the compositions prepared as described above in 96-well microtiter plates to reach a final density of 5*10⁵ cfu/mL. As sterility control, one well containing Mueller-Hinton broth was not inoculated with a microorganism. As growth control, at least 4 wells of the 96-well microtiter plate were prepared, wherein only Mueller-Hinton broth was inoculated with either of the studied microorganisms. In other words, the growth controls were not treated with a composition as referred to herein comprising organic acid(s) or salt(s) thereof and/or any hypophosphite.

The thus inoculated microtiter plates were incubated overnight at 37° C. under anaerobic conditions, provided by a saturated nitrogen atmosphere in anaerobic jars to simulate conditions in a gastrointestinal tract. To evaluate microbial growth, the optical density at 600 nanometers (OD600) of each well of the incubated microtiter plates was measured approximately 20-26 h after incubation start, typically after approximately 24 h, using a multimode plate reader. The initial optical density value prior to overnight incubation was subtracted from the final value obtained after the overnight incubation period to calculate the growth values. These growth values were used to calculate the amount of inhibition compared to the growth values of the growth control wells. For instance, if the growth value of the growth control was 0.8308 after approximately 10 h, the growth value of an E. coli culture comprising 12.5 mM formic acid was 0.6624, the growth value of an E. coli culture comprising 12.5 mM sodium hypophosphite was 0.7128, and the growth value of an E. coli culture comprising 12.5 mM formic acid and 12.5 mM sodium hypophosphite was 0.3692, then the growth of the culture comprising 12.5 mM formic acid compared to the growth of the growth control would be calculated as 0.6624*100/0.8308=79.72%, the growth of the culture comprising 12.5 mM sodium hypophosphite compared to the growth of the growth control would be calculated as 0.7128*100/0.8308=85.80%, and the growth of the culture comprising 12.5 mM formic acid and 12.5 mM sodium hypophosphite compared to the growth of the growth control would be calculated as 0.3692*100/0.8308=44.44%. In other words, the composition comprising 12.5 mM formic acid inhibited the microbial growth by 100−79.92=20.28%, the composition comprising 12.5 mM sodium hypophosphite inhibited the microbial growth by 100−85.80=14.20%, and the composition comprising 12.5 mM formic acid and 12.5 mM sodium hypophosphite inhibited the microbial growth by 100−44.44=55.56%.

Synergistic inhibition was assessed as published by Colby et al. (1967. Weeds 15(1): 20-22) using the following formula:

$$E = X + ( Y / 1 \; 00 ) ⋆ ( 100 - X )$$

Therein, “E” represents an expectation value; “X” represents the degree of inhibition observed upon treatment with a substance 1 (herein: organic acid(s) and/or salt(s) thereof); and “Y” represents the degree of inhibition observed upon treatment with a substance 2 (herein: hypophosphorous acid(s) and/or salt(s) thereof). The expectation value indicates the theoretical amount of inhibition that would be observable in case of merely additive (i.e. non-synergistic) inhibition of a combination of substance 1 and substance 2. Both, “X” and “Y” are calculated in percent of inhibition. A growth control that was not treated with substance 1 and/or substance 2, served as base value having no inhibition, i.e. 0% of inhibition.

In a next step, the actually observed amount of inhibition of a combination of substance 1 and substance 2 was compared to the expectation value, and used to quantify the synergy according to the following formula:

$$S = O - E$$

Therein, “S” represents the difference in the amount of inhibition between the calculated additive inhibition (“E”) and the actually observed amount of inhibition (“O”); “E” represents the expectation value as described above; and “O” denotes the observed amount of inhibition. In the event that “S” is a positive number, i.e. larger than 0, a combination of substance 1 and substance 2 was confirmed to inhibit the growth of the studied microbe synergistically.

For instance, in the example above, wherein a composition comprising 12.5 mM formic acid inhibited the microbial growth by 20.28%, a composition comprising 12.5 mM sodium hypophosphite inhibited the microbial growth by 14.20%, the expectation value “E” would be calculated as 20.28+(14.20/100)*(100−20.28)=31.60%. In other words, in case of an additive or non-synergistic inhibition, a composition comprising both 12.5 mM formic acid and 12.5 mM sodium hypophosphite would be expected to inhibit the microbial growth by 31.60%. However, in contrast to this expectation, the microbial growth was experimentally determined to be inhibited by 55.56%. Thus, “S” would be calculated as 55.56−31.60=23,96%, indicating synergistic inhibition.

Synergistic modulation (or control) of microbial growth, in particular synergistically reduced growth of potential pathogens was found upon treatment with compositions comprising a combination of i) one or more organic acid(s) or salt(s) thereof with ii) hypophosphorous acid(s) or salt(s) thereof. In the following Tables 3-13, examples of synergistic inhibition of microbial growth by compositions according to the invention are shown. Therein, the column labeled “SHP mM”/“MHP mM”/“PHP mM” indicates the concentration of hypophosphite that was comprised in a combination composition as described above. The column labeled “acid/salt mM” indicates the concentration of organic acid(s) or salt(s) thereof that was comprised in a combination composition as described above. The column labeled “details” indicates i) which organic acid, or which combination of organic acids, or which salt(s) thereof was/were comprised in the combination composition. And in addition, the synergistic inhibition effect (“S”) is given in brackets after the organic acid(s) or salt(s) thereof with which the synergistic inhibition was observed in combination with the respective hypophosphite.

TABLE 3
Synergistic inhibition of microbial growth of Escherichia coli U5/41 (DSMZ 30083) upon treatment with different
concentrations of sodium hypophosphite (SHP) in combination with different concentrations of organic acid(s)
or salt(s) thereof. In the details column, the tested organic acid(s) or salt(s) thereof is indicated followed by
the observed “S” value in brackets. Individual experimental results are separated by semicolon(s).

| SHP | acid/salt | |
|---|---|---|
| mM | mM | details |
| 100.00 | 200.00 | calcium formate (3.32); |
| 100.00 | 100.00 | acetic (0.15); calcium formate (15.64); octanoic (8.87); pyruvic (0.2); valeric (0.04); |
| 100.00 | 50.00 | acetic (0.02); calcium formate (26.49); octanoic (8.55); pyruvic (0.31); |
| 100.00 | 25.00 | acetic (0.21); butyric (0.08); calcium formate (16.62); lactic (0.88); octanoic (10.38); pyruvic (1.11); |
| | | succinic (0.42); valeric (0.16); |
| 100.00 | 20.00 | formic + acetic + propionic + benzoic (7.27); |
| 100.00 | 12.50 | acetic (12.27); butyric (4.27); citric (1.35); formic (23.13); fumaric (15.66); lactic (2.39); octanoic (10.72); |
| | | propionic (2.9); succinic (4.14); valeric (3.19); |
| 100.00 | 10.00 | benzoic (1.55); formic + acetic + propionic + benzoic (16.76); |
| 100.00 | 6.25 | acetic (2.91); formic + acetic + propionic (1.18); citric (9.01); formic (16.2); fumaric (13.63); |
| | | octanoic (7.06); succinic (1.93); valeric (1.03); |
| 100.00 | 5.00 | formic + acetic + propionic + benzoic (4.73); |
| 100.00 | 3.13 | citric (8.09); formic (1.9); fumaric (16.99); octanoic (2.67); |
| 100.00 | 1.56 | citric (5.57); fumaric (7.53); octanoic (2.52); succinic (4.91); |
| 100.00 | 0.78 | fumaric (5.1); |
| 50.00 | 100.00 | acetic (0.05); butyric (0.2); formic (0.15); octanoic (10.98); pyruvic (0.03); |
| 50.00 | 50.00 | octanoic (12.48); pyruvic (0.26); |
| 50.00 | 25.00 | acetic (0.13); butyric (0.16); calcium formate (8.88); lactic (1.58); octanoic (14.39); pyruvic (1.05); |
| | | succinic (0.48); |
| 50.00 | 20.00 | formic + acetic + propionic + benzoic (14.74); |
| 50.00 | 12.50 | acetic (13.79); butyric (5.45); citric (0.8); formic (22.34); fumaric (7.22); lactic (8.47); octanoic (11.49); |
| | | propionic (4.79); succinic (1.36); valeric (3.87); |
| 50.00 | 10.00 | formic + acetic + propionic + benzoic (42.99); |
| 50.00 | 6.25 | acetic (1.76); formic + acetic + propionic (1.11); citric (7.86); formic (20.57); lactic (6.21); |
| | | octanoic (5.83); propionic (6.75); |
| 50.00 | 5.00 | formic + acetic + propionic + benzoic (27.69); |
| 50.00 | 5.00 | benzoic (2.59); sorbic (10.69); |
| 50.00 | 3.13 | formic (1.73); fumaric (2.22); lactic (3.18); octanoic (2.55); |
| 50.00 | 2.50 | benzoic (0.16); |
| 50.00 | 1.56 | fumaric (10.16); lactic (3.68); octanoic (2.02); succinic (8.05); |
| 50.00 | 1.25 | formic + acetic + propionic + benzoic (1.95); |
| 50.00 | 0.63 | benzoic (0.1); |
| 50.00 | 0.39 | fumaric (7.19); |
| 50.00 | 0.31 | benzoic (5.49); |
| 25.00 | 100.00 | acetic (0.01); formic (0.03); octanoic (11.64); pyruvic (0.24); valeric (0.04); |
| 25.00 | 50.00 | acetic (0.13); calcium formate (5.45); octanoic (14.56); valeric (0.01); |
| 25.00 | 25.00 | acetic (0.37); calcium formate (19.23); lactic (0.09); octanoic (15.91); pyruvic (0.81); succinic (0.1); |
| 25.00 | 20.00 | formic + acetic + propionic + benzoic (18.17); |
| 25.00 | 12.50 | acetic (14.4); butyric (5.53); calcium formate (12.62); citric (1.06); formic (14.97); fumaric (9.58); |
| | | lactic (3.64); octanoic (11.92); propionic (4.31); succinic (3.17); valeric (3.78); |
| 25.00 | 10.00 | formic + acetic + propionic + benzoic (51.83); |
| 25.00 | 6.25 | acetic (5.58); citric (11.1); formic (20.78); sodium benzoate (5.05); octanoic (3.04); |
| 25.00 | 5.00 | sorbic (8.39); formic + acetic + propionic + benzoic (11.75); |
| 25.00 | 3.13 | acetic (0.67); citric (0.97); octanoic (3.72); succinic (1.67); |
| 25.00 | 1.56 | octanoic (1.76); |
| 25.00 | 0.78 | fumaric (7.3); |
| 12.50 | 100.00 | acetic (0.01); formic (0.15); octanoic (15.76); pyruvic (0.21); |
| 12.50 | 50.00 | acetic (0.09); octanoic (16.7); pyruvic (0.15); |
| 12.50 | 25.00 | calcium formate (13.66); lactic (0.71); octanoic (19.51); pyruvic (0.97); succinic (0.14); |
| 12.50 | 20.00 | formic + acetic + propionic + benzoic (25.12); |
| 12.50 | 12.50 | acetic (14.96); butyric (4.74); calcium formate (7.73); citric (1.82); formic (21.79); fumaric (9.1); |
| | | lactic (5.48); octanoic (8.64); propionic (4.45); valeric (4.76); |
| 12.50 | 10.00 | benzoic (2.35); sorbic (0.44); formic + acetic + propionic + benzoic (74.6); |
| 12.50 | 6.25 | acetic (9.16); citric (12.77); formic (23.1); sodium benzoate (11.28); octanoic (6.44); |
| 12.50 | 5.00 | benzoic (3.07); sorbic (5.08); |
| 12.50 | 3.13 | citric (3.77); formic (7.14); fumaric (9.11); octanoic (4.9); succinic (2.78); |
| 12.50 | 1.56 | citric (8.28); formic (3.38); octanoic (4.64); succinic (6.99); |
| 12.50 | 0.78 | fumaric (5.2); |
| 12.50 | 0.39 | fumaric (7.2); |
| 12.50 | 0.31 | benzoic (0.42); |
| 6.25 | 100.00 | formic (0.03); octanoic (16.62); pyruvic (0.33); |
| 6.25 | 80.00 | formic + acetic + propionic + benzoic (0.73); |
| 6.25 | 50.00 | octanoic (18.98); |
| 6.25 | 25.00 | acetic (0.26); calcium formate (9.43); lactic (2.01); octanoic (20.21); pyruvic (0.35); |
| 6.25 | 20.00 | formic + acetic + propionic + benzoic (30.38); |
| 6.25 | 12.50 | acetic (15.16); butyric (5.82); calcium formate (6.97); citric (1.36); formic (21.67); lactic (15.15); |
| | | sodium benzoate (0.01); octanoic (10.58); propionic (4.35); succinic (3.92); valeric (4.58); |
| 6.25 | 10.00 | benzoic (2.31); sorbic (0.28); formic + acetic + propionic + benzoic (53.06); |
| 6.25 | 6.25 | acetic (9.89); citric (14); formic (9.79); lactic (5.23); sodium benzoate (13.39); octanoic (6.65); |
| 6.25 | 5.00 | benzoic (6.43); sorbic (11.24); formic + acetic + propionic + benzoic (14.27); |
| 6.25 | 3.13 | citric (6.98); formic (5.04); fumaric (8.81); octanoic (3.27); succinic (8.72); |
| 6.25 | 2.50 | benzoic (2.95); formic + acetic + propionic + benzoic (5.49); |
| 6.25 | 1.56 | citric (1.34); lactic (0.99); octanoic (5.28); succinic (6.52); |
| 6.25 | 1.25 | formic + acetic + propionic + benzoic (7.65); |
| 6.25 | 1.25 | benzoic (0.98); |
| 6.25 | 0.78 | fumaric (18.57); |
| 6.25 | 0.63 | benzoic (0.41); |
| 6.25 | 0.31 | benzoic (1.44); |
| 3.13 | 100.00 | octanoic (19.07); pyruvic (0.27); valeric (0.12); |
| 3.13 | 80.00 | formic + acetic + propionic + benzoic (0.93); |
| 3.13 | 50.00 | octanoic (21.16); |
| 3.13 | 25.00 | lactic (1.74); octanoic (22.36); pyruvic (1.86); succinic (0.73); |
| 3.13 | 20.00 | formic + acetic + propionic + benzoic (32.14); |
| 3.13 | 12.50 | acetic (14.43); butyric (5.47); calcium formate (0.67); citric (1.04); formic (20.64); fumaric (14.88); |
| | | lactic (11.37); octanoic (10.14); propionic (5.37); succinic (4.63); valeric (4.66); |
| 3.13 | 10.00 | formic + acetic + propionic + benzoic (55.93); |
| 3.13 | 6.25 | acetic (4.96); citric (12.43); formic (4.5); fumaric (8.65); lactic (7.05); octanoic (5.66); propionic (1.06); |
| 3.13 | 5.00 | formic + acetic + propionic + benzoic (8.88); |
| 3.13 | 3.13 | citric (6.44); formic (2.53); fumaric (13.81); lactic (0.63); octanoic (4.51); pyruvic (2.71); succinic (11.92); |
| 3.13 | 2.50 | formic + acetic + propionic + benzoic (6.25); |
| 3.13 | 1.56 | citric (2.87); fumaric (4.87); octanoic (3.26); succinic (8.84); |
| 3.13 | 1.25 | formic + acetic + propionic + benzoic (11.53); |
| 1.56 | 100.00 | octanoic (23.32); pyruvic (0.46); valeric (0.01); |
| 1.56 | 80.00 | formic + acetic + propionic + benzoic (1.16); |
| 1.56 | 50.00 | octanoic (25.76); |
| 1.56 | 25.00 | lactic (2.59); octanoic (27.03); pyruvic (1.46); succinic (0.89); |
| 1.56 | 20.00 | formic + acetic + propionic + benzoic (33.13); |
| 1.56 | 12.50 | acetic (13.39); butyric (6.92); citric (1.03); formic (20.87); fumaric (15.97); lactic (17.1); octanoic (6.44); |
| | | propionic (6.57); succinic (5.33); valeric (5.66); |
| 1.56 | 10.00 | formic + acetic + propionic + benzoic (50.52); |
| 1.56 | 6.25 | acetic (9.74); citric (12.1); formic (5.66); fumaric (4.99); lactic (6.98); octanoic (5.06); propionic (4.09); |
| 1.56 | 5.00 | formic + acetic + propionic + benzoic (7.73); |
| 1.56 | 3.13 | citric (6.12); formic (4.22); fumaric (17.34); lactic (9.52); octanoic (9.38); pyruvic (1.19); succinic (7.82); |
| 1.56 | 2.50 | benzoic (2.69); formic + acetic + propionic + benzoic (8.15); |
| 1.56 | 1.56 | citric (2.55); formic (0.16); fumaric (11.51); lactic (1.79); octanoic (7.75); propionic (1.56); succinic (6.02); |
| 1.56 | 1.25 | benzoic (1.45); formic + acetic + propionic + benzoic (12.8); |
| 1.56 | 0.63 | benzoic (1.21); |
| 1.56 | 0.39 | fumaric (3.98); |
| 1.56 | 0.31 | benzoic (0.58); |
| 0.78 | 100.00 | butyric (0.05); formic (0.16); octanoic (20.56); pyruvic (0.45); |
| 0.78 | 80.00 | formic + acetic + propionic + benzoic (1.15); |
| 0.78 | 50.00 | octanoic (23.96); pyruvic (0.02); |
| 0.78 | 40.00 | formic + acetic + propionic + benzoic (0.01); |
| 0.78 | 25.00 | acetic (0.14); lactic (2.71); octanoic (24.4); pyruvic (1.87); succinic (0.62); |
| 0.78 | 20.00 | formic + acetic + propionic + benzoic (33.81); |
| 0.78 | 12.50 | acetic (13.73); butyric (7.83); citric (2.81); formic (18.31); fumaric (17.65); lactic (21.71); octanoic (2.09); |
| | | propionic (6.32); succinic (5.53); valeric (6.11); |
| 0.78 | 10.00 | benzoic (2.44); sorbic (2.01); formic + acetic + propionic + benzoic (47.22); |
| 0.78 | 6.25 | acetic (5.51); citric (21.71); formic (0.62); fumaric (11.48); lactic (10.86); octanoic (1.44); propionic (3); |
| 0.78 | 5.00 | formic + acetic + propionic + benzoic (6.5); |
| 0.78 | 3.13 | acetic (2.12); citric (13.95); formic (0.69); fumaric (14.47); lactic (6.03); pyruvic (2.62); succinic (5.72); |
| 0.78 | 2.50 | benzoic (5.51); formic + acetic + propionic + benzoic (6.6); |
| 0.78 | 1.56 | citric (7.56); fumaric (2.29); lactic (2.08); octanoic (0.65); pyruvic (1.23); succinic (2.9); |
| 0.78 | 1.25 | benzoic (2.51); formic + acetic + propionic + benzoic (12.42); |
| 0.78 | 0.78 | fumaric (1.85); |
| 0.78 | 0.63 | benzoic (0.35); |
| 0.78 | 0.31 | benzoic (2.22); |
| 0.39 | 100.00 | butyric (0.08); formic (0.24); octanoic (24.71); succinic (0.05); |
| 0.39 | 80.00 | formic + acetic + propionic + benzoic (1.54); |
| 0.39 | 50.00 | octanoic (24.85); pyruvic (0.09); |
| 0.39 | 40.00 | formic + acetic + propionic + benzoic (0.57); |
| 0.39 | 25.00 | acetic (0.06); formic + acetic + propionic (0.17); lactic (1.88); octanoic (28.25); pyruvic (0.81); |
| 0.39 | 20.00 | formic + acetic + propionic + benzoic (35.78); |
| 0.39 | 12.50 | acetic (8.75); butyric (4.21); citric (3.35); formic (12.97); fumaric (18.25); lactic (21.17); octanoic (1.39); |
| | | propionic (7.47); succinic (2.61); valeric (5.29); |
| 0.39 | 10.00 | benzoic (1.27); sorbic (1.6); formic + acetic + propionic + benzoic (57.11); |
| 0.39 | 6.25 | formic + acetic + propionic (0.16); citric (23.43); fumaric (9.91); lactic (9.36); octanoic (2.91); |
| 0.39 | 5.00 | benzoic (3.43); formic + acetic + propionic + benzoic (9.27); |
| 0.39 | 3.13 | citric (16.12); fumaric (12.05); lactic (4.7); octanoic (7.27); succinic (0.95); |
| 0.39 | 2.50 | benzoic (4.97); formic + acetic + propionic + benzoic (10.36); |
| 0.39 | 1.56 | citric (9.63); formic (0.34); fumaric (6.54); lactic (1.83); octanoic (8.66); succinic (1.9); |
| 0.39 | 1.25 | benzoic (2.82); formic + acetic + propionic + benzoic (15.47); |
| 0.39 | 0.63 | benzoic (3.7); |
| 0.39 | 0.39 | fumaric (0.06); |
| 0.39 | 0.31 | benzoic (3.12); |
| 0.20 | 100.00 | butyric (0.09); formic (0.13); octanoic (23.7); pyruvic (1.24); |
| 0.20 | 80.00 | formic + acetic + propionic + benzoic (1.02); |
| 0.20 | 50.00 | acetic (0.16); formic + acetic + propionic (0.29); octanoic (26.27); |
| 0.20 | 40.00 | formic + acetic + propionic + benzoic (0.91); |
| 0.20 | 25.00 | acetic (0.09); octanoic (13.53); pyruvic (2.25); valeric (0.05); |
| 0.20 | 20.00 | formic + acetic + propionic + benzoic (35.64); |
| 0.20 | 12.50 | acetic (10.12); citric (3.95); fumaric (16.5); lactic (16.95); octanoic (3.93); propionic (6.18); valeric (0.12); |
| 0.20 | 10.00 | formic + acetic + propionic + benzoic (38.03); |
| 0.20 | 6.25 | formic + acetic + propionic (0.42); citric (22.95); formic (2.23); fumaric (1.41); lactic (7.26); |
| 0.20 | 5.00 | formic + acetic + propionic + benzoic (9.59); |
| 0.20 | 3.13 | formic + acetic + propionic (2.16); citric (9.6); formic (2.64); fumaric (8.08); lactic (5.97); octanoic (5.99); |
| | | pyruvic (9.74); |
| 0.20 | 2.50 | formic + acetic + propionic + benzoic (9.47); |
| 0.20 | 1.56 | citric (5.61); formic (2); fumaric (1.56); lactic (1.12); octanoic (0.49); pyruvic (4.76); valeric (4.23); |
| 0.20 | 1.25 | benzoic (4.01); formic + acetic + propionic + benzoic (16.52); |
| 0.20 | 0.78 | fumaric (2.76); |
| 0.20 | 0.63 | benzoic (2.95); |
| 0.20 | 0.39 | fumaric (4.48); |
| 0.20 | 0.31 | benzoic (4.07); |

TABLE 4
Synergistic inhibition of microbial growth of Escherichia coli U5/41 (DSMZ 30083) upon treatment with different
concentrations of magnesium hypophosphite (MHP) in combination with different concentrations of organic
acid(s) or salt(s) thereof. In the details column, the tested organic acid(s) or salt(s) thereof is indicated followed
by the observed “S” value in brackets. Individual experimental results are separated by semicolon(s).

| MHP | acid/salt | |
|---|---|---|
| mM | mM | details |
| 100.00 | 100.00 | calcium formate (19.84); |
| 100.00 | 50.00 | calcium formate (18.84); |
| 100.00 | 25.00 | calcium formate (11.47); |
| 100.00 | 20.00 | formic + acetic + propionic + benzoic (8.04); |
| 100.00 | 12.50 | formic + acetic + propionic (1.09 calcium formate (11.63); citric (1.77); |
| 100.00 | 10.00 | formic + acetic + propionic + benzoic (20.8); |
| 100.00 | 6.25 | formic + acetic + propionic (5.78); calcium formate (3.83); formic (8.87); lactic (7.62); |
| 100.00 | 5.00 | formic + acetic + propionic + benzoic (7.16); |
| 100.00 | 3.13 | acetic (3.99); formic (6.69); lactic (8.95); |
| 100.00 | 1.56 | acetic (4.91); formic (7.96); lactic (6.63); |
| 50.00 | 50.00 | calcium formate (5.13); |
| 50.00 | 25.00 | calcium formate (5.36); |
| 50.00 | 20.00 | formic + acetic + propionic + benzoic (9.98); |
| 50.00 | 12.50 | formic + acetic + propionic (0.9); calcium formate (10.65); citric (3.78); |
| 50.00 | 10.00 | formic + acetic + propionic + benzoic (22.9); |
| 50.00 | 6.25 | formic + acetic + propionic (2.09); calcium formate (5.33); formic (5.96); |
| 50.00 | 5.00 | formic + acetic + propionic + benzoic (7.35); |
| 50.00 | 3.13 | acetic (5.54); formic (5.7); propionic (2.57); |
| 50.00 | 1.56 | acetic (4.89); formic (6.14); propionic (5.7); |
| 50.00 | 0.63 | benzoic (0.45); |
| 50.00 | 0.31 | benzoic (3.75); |
| 25.00 | 100.00 | citric (0.06); |
| 25.00 | 25.00 | |
| 25.00 | 20.00 | formic + acetic + propionic + benzoic (14.35); |
| 25.00 | 12.50 | formic + acetic + propionic (0.94); calcium formate (8.47); citric (3.59); sodium benzoate (1.53); |
| 25.00 | 10.00 | formic + acetic + propionic + benzoic (35.69); |
| 25.00 | 6.25 | calcium formate (0.73); formic (6.38); sodium benzoate (4.56); |
| 25.00 | 5.00 | formic + acetic + propionic + benzoic (17.49); |
| 25.00 | 3.13 | acetic (3.52); formic (1.46); propionic (5.32); |
| 25.00 | 1.56 | acetic (6.09); formic (4.02); lactic (6.32); propionic (7.28); |
| 25.00 | 0.31 | benzoic (8.5); |
| 12.50 | 50.00 | sodium benzoate (2.05); |
| 12.50 | 25.00 | sodium benzoate (0.53); |
| 12.50 | 20.00 | formic + acetic + propionic + benzoic (24.33); |
| 12.50 | 12.50 | formic + acetic + propionic (4.04); calcium formate (2.26); citric (3.4); sodium benzoate (8.49); |
| 12.50 | 10.00 | formic + acetic + propionic + benzoic (64); |
| 12.50 | 6.25 | formic (2.24); sodium benzoate (13.45); |
| 12.50 | 5.00 | formic + acetic + propionic + benzoic (9.7); |
| 12.50 | 3.13 | acetic (3.37); formic (2.18); |
| 12.50 | 1.56 | acetic (1.55); formic (2.32); lactic (1.32); |
| 6.25 | 100.00 | sodium benzoate (0.06); |
| 6.25 | 80.00 | formic + acetic + propionic + benzoic (0.45); |
| 6.25 | 50.00 | sodium benzoate (2.18); |
| 6.25 | 40.00 | formic + acetic + propionic + benzoic (0.02); |
| 6.25 | 25.00 | sodium benzoate (1.78); |
| 6.25 | 20.00 | formic + acetic + propionic + benzoic (31.53); |
| 6.25 | 12.50 | formic + acetic + propionic (9.92); calcium formate (6.11); citric (3.36); sodium benzoate (9.25); |
| 6.25 | 10.00 | formic + acetic + propionic + benzoic (76.19); |
| 6.25 | 6.25 | sodium benzoate (15.12); |
| 6.25 | 5.00 | formic + acetic + propionic + benzoic (16.33); |
| 6.25 | 1.56 | acetic (2.55); lactic (1.41); |
| 3.13 | 100.00 | acetic (0.04); |
| 3.13 | 80.00 | formic + acetic + propionic + benzoic (0.8); |
| 3.13 | 40.00 | formic + acetic + propionic + benzoic (0.61); |
| 3.13 | 20.00 | formic + acetic + propionic + benzoic (33.46); |
| 3.13 | 12.50 | calcium formate (0.3); citric (3.6); |
| 3.13 | 10.00 | formic + acetic + propionic + benzoic (58.6); |
| 3.13 | 6.25 | acetic (1.17); formic (4.24); lactic (10.62); |
| 3.13 | 5.00 | benzoic (18.1); formic + acetic + propionic + benzoic (10.71); |
| 3.13 | 3.13 | acetic (33.89); formic (12.43); lactic (23.13); propionic (15.71); |
| 3.13 | 1.56 | lactic (0.01); |
| 3.13 | 1.25 | benzoic (14.99); |
| 3.13 | 0.63 | benzoic (55.02); |
| 3.13 | 0.31 | benzoic (8.51); |
| 1.56 | 80.00 | formic + acetic + propionic + benzoic (0.97); |
| 1.56 | 40.00 | formic + acetic + propionic + benzoic (0.49); |
| 1.56 | 20.00 | formic + acetic + propionic + benzoic (34.72); |
| 1.56 | 20.00 | benzoic (0.04); |
| 1.56 | 12.50 | formic + acetic + propionic (3.1); citric (3.91); |
| 1.56 | 10.00 | formic + acetic + propionic + benzoic (61.74); |
| 1.56 | 6.25 | formic (2.84); lactic (9.7); |
| 1.56 | 5.00 | benzoic (21.08); formic + acetic + propionic + benzoic (7.65); |
| 1.56 | 3.13 | lactic (4.78); |
| 1.56 | 2.50 | formic + acetic + propionic + benzoic (0.36); |
| 1.56 | 1.56 | lactic (9.15); |
| 1.56 | 1.25 | benzoic (5.31); formic + acetic + propionic + benzoic (0.44); |
| 1.56 | 0.63 | benzoic (7.02); |
| 1.56 | 0.31 | benzoic (6.28); |
| 0.78 | 100.00 | formic (0.11); |
| 0.78 | 80.00 | formic + acetic + propionic + benzoic (0.85); |
| 0.78 | 40.00 | formic + acetic + propionic + benzoic (0.54); |
| 0.78 | 25.00 | formic (0); lactic (0.11); |
| 0.78 | 20.00 | benzoic (0.13); formic + acetic + propionic + benzoic (35.4); |
| 0.78 | 12.50 | formic + acetic + propionic (6.1); citric (4.07); |
| 0.78 | 10.00 | benzoic (6.05); formic + acetic + propionic + benzoic (55.81); |
| 0.78 | 6.25 | calcium formate (1.58); formic (1.62); lactic (20.06); propionic (0.78); |
| 0.78 | 5.00 | benzoic (23.02); formic + acetic + propionic + benzoic (5.43); |
| 0.78 | 3.13 | acetic (3.5); formic (1.61); lactic (18.37); |
| 0.78 | 1.56 | acetic (34.81); formic (26.94); lactic (41.1); propionic (29.56); |
| 0.78 | 1.25 | benzoic (5.15); formic + acetic + propionic + benzoic (1.17); |
| 0.78 | 0.63 | benzoic (11.58); |
| 0.78 | 0.31 | benzoic (62.65); |
| 0.39 | 80.00 | formic + acetic + propionic + benzoic (1.05); |
| 0.39 | 50.00 | formic + acetic + propionic (0.41); |
| 0.39 | 40.00 | formic + acetic + propionic + benzoic (1.08); |
| 0.39 | 25.00 | formic + acetic + propionic (0.52); lactic (0.11); |
| 0.39 | 20.00 | formic + acetic + propionic + benzoic (36.74); |
| 0.39 | 12.50 | formic + acetic + propionic (10.33); calcium formate (9.21); citric (4.26); |
| 0.39 | 10.00 | formic + acetic + propionic + benzoic (55.14); |
| 0.39 | 6.25 | calcium formate (0.98); lactic (15.95); |
| 0.39 | 5.00 | benzoic (16.22); formic + acetic + propionic + benzoic (5.54); |
| 0.39 | 3.13 | lactic (3.2); |
| 0.39 | 2.50 | formic + acetic + propionic + benzoic (1.19); |
| 0.39 | 1.56 | lactic (5.42); |
| 0.39 | 1.25 | formic + acetic + propionic + benzoic (1.2); |
| 0.39 | 0.63 | benzoic (2.35); |
| 0.39 | 0.31 | benzoic (5.23); |
| 0.20 | 400.00 | calcium formate (2.14); |
| 0.20 | 100.00 | formic + acetic + propionic (0.24); propionic (0.16); |
| 0.20 | 80.00 | formic + acetic + propionic + benzoic (1.1); |
| 0.20 | 50.00 | acetic (0.04); formic + acetic + propionic (1.02); |
| 0.20 | 40.00 | formic + acetic + propionic + benzoic (1.29); |
| 0.20 | 25.00 | formic + acetic + propionic (0.61); |
| 0.20 | 20.00 | benzoic (0.16); formic + acetic + propionic + benzoic (36.78); |
| 0.20 | 12.50 | formic + acetic + propionic (8.53); calcium formate (2.99); citric (4.82); |
| 0.20 | 10.00 | formic + acetic + propionic + benzoic (36.6); |
| 0.20 | 6.25 | acetic (0.4); formic + acetic + propionic (2.84); lactic (9.62); propionic (0.26); |
| 0.20 | 5.00 | benzoic (13.01); formic + acetic + propionic + benzoic (6.07); |
| 0.20 | 3.13 | acetic (2.24); formic + acetic + propionic (1.85); formic (6.02); propionic (5.49); |
| 0.20 | 2.50 | formic + acetic + propionic + benzoic (0.31); |
| 0.20 | 1.56 | formic (5.91); |
| 0.20 | 1.25 | formic + acetic + propionic + benzoic (4.58); |
| 0.20 | 0.31 | benzoic (4.84); |

TABLE 5
Synergistic inhibition of microbial growth of Escherichia coli U5/41 (DSMZ 30083) upon treatment with different concentra-
tions of potassium hypophosphite (PHP) in combination with different concentrations of organic acid(s) or salt(s) thereof. In the
details column, the tested organic acid(s) or salt(s) thereof is indicated followed by the observed “S” value in brackets. Individual
experimental results are separated by semicolon(s).

| PHP | acid/salt | |
|---|---|---|
| mM | mM | details |
| 100.00 | 100.00 | acetic (0.02); sodium benzoate (1.09); octanoic (5.95); |
| 100.00 | 50.00 | butyric (0.07); octanoic (5.36); succinic (0.06); |
| 100.00 | 25.00 | butyric (0.07); octanoic (7.47); succinic (0.74); |
| 100.00 | 20.00 | formic + acetic + propionic + benzoic (6.13); |
| 100.00 | 12.50 | acetic (1); butyric (1.09); formic (9.55); fumaric (6.91); lactic (1.18); octanoic (6.62); propionic (1.77); |
| | | succinic (7.86); |
| 100.00 | 10.00 | sorbic (2.86); formic + acetic + propionic + benzoic (13.38); |
| 100.00 | 6.25 | acetic (1.69); butyric (1.96); formic + acetic + propionic (2.14); formic (9.5); fumaric (10.2); lactic (7.65); |
| | | succinic (2.5); |
| 100.00 | 5.00 | benzoic (0.74); sorbic (6.8); |
| 100.00 | 3.13 | lactic (0.26); |
| 100.00 | 2.50 | sorbic (9.8); |
| 100.00 | 1.56 | formic (5.58); lactic (5.18); succinic (0.74); |
| 100.00 | 1.25 | sorbic (1.09); |
| 100.00 | 0.63 | benzoic (6); |
| 100.00 | 0.31 | benzoic (3.39); |
| 50.00 | 100.00 | acetic (0); butyric (0.11); octanoic (6.33); succinic (0.06); |
| 50.00 | 50.00 | octanoic (5.7); succinic (0.05); |
| 50.00 | 25.00 | butyric (0.1); octanoic (8.33); succinic (0.68); |
| 50.00 | 20.00 | formic + acetic + propionic + benzoic (14.62); |
| 50.00 | 12.50 | acetic (4.11); butyric (0.75); formic (8.36); fumaric (6.72); octanoic (5.27); propionic (3.78); succinic |
| | | (8.14); |
| 50.00 | 10.00 | formic + acetic + propionic + benzoic (45.31); |
| 50.00 | 6.25 | acetic (4.12); formic (6.85); fumaric (5.2); succinic (2.84); valeric (0.14); |
| 50.00 | 5.00 | benzoic (1.45); sorbic (7.48); formic + acetic + propionic + benzoic (32.58); |
| 50.00 | 3.13 | acetic (5.89); |
| 50.00 | 2.50 | sorbic (9.49); |
| 50.00 | 1.56 | lactic (2.95); succinic (1.02); |
| 50.00 | 1.25 | sorbic (1.2); |
| 50.00 | 0.63 | sorbic (0.53); |
| 50.00 | 0.31 | benzoic (12.56); |
| 25.00 | 100.00 | sodium benzoate (1.35); octanoic (6.01); propionic (0.06); valeric (0.03); |
| 25.00 | 50.00 | butyric (0.02); sodium benzoate (3.51); octanoic (5.33); succinic (0.02); |
| 25.00 | 25.00 | octanoic (8.76); succinic (0.48); |
| 25.00 | 20.00 | formic + acetic + propionic + benzoic (23.58); |
| 25.00 | 12.50 | acetic (3.76); butyric (0.1); formic + acetic + propionic (2.81); formic (12.18); fumaric (7.06); sodium |
| | | benzoate (4.21); octanoic (3.02); propionic (3.59); succinic (7.27); |
| 25.00 | 10.00 | sorbic (2.96); formic + acetic + propionic + benzoic (61.1); |
| 25.00 | 6.25 | acetic (2.79); formic (16.12); fumaric (7.36); sodium benzoate (8.09); succinic (1.63); |
| 25.00 | 5.00 | sorbic (8.09); formic + acetic + propionic + benzoic (21.87); |
| 25.00 | 3.13 | acetic (1.1); formic (2.98); fumaric (0.82); |
| 25.00 | 2.50 | sorbic (6.48); |
| 25.00 | 1.56 | formic (1.1); |
| 25.00 | 1.25 | sorbic (1.13); |
| 25.00 | 0.63 | benzoic (3.29); sorbic (0.54); |
| 25.00 | 0.31 | benzoic (3.14); sorbic (0.17); |
| 25.00 | 0.16 | sorbic (0.06); |
| 12.50 | 100.00 | formic (0.02); sodium benzoate (1.73); octanoic (6.69); |
| 12.50 | 50.00 | formic (0.08); sodium benzoate (6.5); octanoic (5.84); |
| 12.50 | 25.00 | octanoic (9.16); succinic (0.53); |
| 12.50 | 20.00 | formic + acetic + propionic + benzoic (24.93); |
| 12.50 | 12.50 | acetic (3.41); formic + acetic + propionic (4.31); formic (8.28); fumaric (7.3); sodium benzoate (7.2); |
| | | octanoic (2.49); propionic (3.4); succinic (7.32); |
| 12.50 | 10.00 | formic + acetic + propionic + benzoic (66.35); |
| 12.50 | 6.25 | formic (12.28); fumaric (5.91); sodium benzoate (3.43); succinic (3.07); |
| 12.50 | 5.00 | benzoic (5.38); sorbic (8.76); |
| 12.50 | 3.13 | formic (1.25); fumaric (1.32); succinic (1.15); |
| 12.50 | 2.50 | sorbic (8.53); |
| 12.50 | 1.56 | succinic (1.51); |
| 12.50 | 1.25 | sorbic (3.08); |
| 12.50 | 0.63 | benzoic (8.13); sorbic (1.22); |
| 12.50 | 0.31 | benzoic (2.75); sorbic (0.92); |
| 12.50 | 0.16 | sorbic (1.19); |
| 6.25 | 200.00 | calcium formate (3.95); |
| 6.25 | 100.00 | formic (0); sodium benzoate (1); octanoic (6.83); succinic (0.11); |
| 6.25 | 50.00 | sodium benzoate (5.58); octanoic (6.49); |
| 6.25 | 25.00 | octanoic (9.71); succinic (0.34); |
| 6.25 | 20.00 | formic + acetic + propionic + benzoic (31.32); |
| 6.25 | 12.50 | acetic (4.29); formic + acetic + propionic (15.11); formic (4.39); fumaric (6.24); sodium benzoate (8.94); |
| | | octanoic (0.87); propionic (3.36); succinic (7.28); |
| 6.25 | 10.00 | sorbic (3.27); formic + acetic + propionic + benzoic (55.94); |
| 6.25 | 6.25 | formic (1.98); fumaric (4.31); sodium benzoate (6.33); |
| 6.25 | 5.00 | benzoic (7.41); sorbic (8.62); formic + acetic + propionic + benzoic (12.98); |
| 6.25 | 3.13 | fumaric (2.5); succinic (3.37); |
| 6.25 | 2.50 | sorbic (7.92); |
| 6.25 | 1.56 | fumaric (0.21); succinic (3.02); |
| 6.25 | 1.25 | formic + acetic + propionic + benzoic (0.32); |
| 6.25 | 0.78 | fumaric (0.82); |
| 6.25 | 0.63 | benzoic (10.8); sorbic (1.47); |
| 6.25 | 0.31 | benzoic (11.62); sorbic (1.63); |
| 3.13 | 200.00 | calcium formate (7.93); |
| 3.13 | 100.00 | octanoic (5.98); valeric (0.02); |
| 3.13 | 80.00 | formic + acetic + propionic + benzoic (0.63); |
| 3.13 | 50.00 | octanoic (5.81); |
| 3.13 | 25.00 | octanoic (9.32); succinic (0.17); |
| 3.13 | 20.00 | formic + acetic + propionic + benzoic (31.37); |
| 3.13 | 12.50 | acetic (3.65); formic + acetic + propionic (5.87); formic (6.5); fumaric (6.57); octanoic (1.2); propionic (3.6); |
| | | succinic (2.66); |
| 3.13 | 10.00 | formic + acetic + propionic + benzoic (50.71); |
| 3.13 | 6.25 | fumaric (2.83); |
| 3.13 | 5.00 | sorbic (8.98); formic + acetic + propionic + benzoic (3.64); |
| 3.13 | 3.13 | fumaric (4.24); lactic (1.73); succinic (0.56); |
| 3.13 | 2.50 | sorbic (10.03); |
| 3.13 | 1.56 | citric (8.05); formic (0.1); fumaric (1.56); lactic (12.78); succinic (1.7); |
| 3.13 | 1.25 | benzoic (2.36); sorbic (3.76); formic + acetic + propionic + benzoic (1.49); |
| 3.13 | 0.78 | fumaric (0.81); |
| 3.13 | 0.63 | benzoic (15.23); sorbic (3.52); |
| 3.13 | 0.31 | benzoic (15.11); sorbic (1.69); |
| 3.13 | 0.16 | sorbic (2.39); |
| 1.56 | 200.00 | calcium formate (4.28); |
| 1.56 | 100.00 | octanoic (6.97); succinic (0.04); |
| 1.56 | 80.00 | formic + acetic + propionic + benzoic (0.16); |
| 1.56 | 50.00 | octanoic (5.49); |
| 1.56 | 25.00 | octanoic (7.64); succinic (0.99); |
| 1.56 | 20.00 | formic + acetic + propionic + benzoic (31.02); |
| 1.56 | 12.50 | acetic (2.66); formic + acetic + propionic (9.86); formic (6.37); fumaric (7.48); octanoic (0.1); propionic |
| | | (3.91); succinic (7.53); |
| 1.56 | 10.00 | sorbic (3.5); formic + acetic + propionic + benzoic (53.05); |
| 1.56 | 6.25 | fumaric (4.27); succinic (4.89); |
| 1.56 | 5.00 | benzoic (3.79); sorbic (9.67); formic + acetic + propionic + benzoic (4.98); |
| 1.56 | 3.13 | formic (3.48); fumaric (4.64); succinic (6.03); |
| 1.56 | 2.50 | sorbic (9.52); |
| 1.56 | 1.56 | formic (2.77); fumaric (2.82); lactic (2.73); succinic (3.88); |
| 1.56 | 1.25 | sorbic (2.49); formic + acetic + propionic + benzoic (6.33); |
| 1.56 | 0.78 | fumaric (3.27); |
| 1.56 | 0.63 | benzoic (8.75); sorbic (4.93); |
| 1.56 | 0.39 | fumaric (0.25); |
| 1.56 | 0.31 | benzoic (9.73); sorbic (3.11); |
| 1.56 | 0.16 | sorbic (1.46); |
| 0.78 | 200.00 | calcium formate (4.41); |
| 0.78 | 100.00 | octanoic (6.91); |
| 0.78 | 80.00 | formic + acetic + propionic + benzoic (0.75); |
| 0.78 | 50.00 | octanoic (7.62); |
| 0.78 | 25.00 | octanoic (8.63); succinic (0.59); |
| 0.78 | 20.00 | formic + acetic + propionic + benzoic (35.08); |
| 0.78 | 12.50 | acetic (3.75); formic + acetic + propionic (15.19); formic (8.2); fumaric (7.43); propionic (4.07); succinic |
| | | (8.55); |
| 0.78 | 10.00 | formic + acetic + propionic + benzoic (43.35); |
| 0.78 | 6.25 | formic (1.01); fumaric (4.85); succinic (4.5); |
| 0.78 | 5.00 | benzoic (11.38); sorbic (10.53); formic + acetic + propionic + benzoic (4.99); |
| 0.78 | 3.13 | formic (1.58); fumaric (3.27); succinic (6.06); |
| 0.78 | 2.50 | sorbic (8.68); formic + acetic + propionic + benzoic (1.58); |
| 0.78 | 1.56 | formic (2.77); fumaric (2.46); lactic (5.1); succinic (5.36); |
| 0.78 | 1.25 | benzoic (3.27); sorbic (3.46); formic + acetic + propionic + benzoic (6.48); |
| 0.78 | 0.78 | fumaric (2.45); |
| 0.78 | 0.63 | benzoic (10.45); sorbic (5.27); |
| 0.78 | 0.39 | fumaric (0.73); |
| 0.78 | 0.31 | benzoic (9.47); sorbic (3.36); |
| 0.78 | 0.16 | sorbic (2.09); |
| 0.39 | 100.00 | formic + acetic + propionic (0.31); formic (0.06); octanoic (4.8); succinic (0.11); |
| 0.39 | 80.00 | formic + acetic + propionic + benzoic (0.8); |
| 0.39 | 50.00 | formic + acetic + propionic (0.16); formic (0.15); octanoic (4.38); |
| 0.39 | 40.00 | formic + acetic + propionic + benzoic (0.35); |
| 0.39 | 25.00 | formic + acetic + propionic (0.63); formic (0.01); fumaric (0.05); octanoic (4.03); succinic (0.63); |
| 0.39 | 20.00 | formic + acetic + propionic + benzoic (36.55); |
| 0.39 | 12.50 | acetic (3.12); formic + acetic + propionic (22.98); fumaric (5.17); propionic (4.26); succinic (7.22); |
| 0.39 | 10.00 | sorbic (3.67); formic + acetic + propionic + benzoic (47.76); |
| 0.39 | 6.25 | formic + acetic + propionic (0.75); formic (2.1); fumaric (6.71); succinic (1.55); valeric (0.68); |
| 0.39 | 5.00 | benzoic (12.28); sorbic (10.1); formic + acetic + propionic + benzoic (7.47); |
| 0.39 | 3.13 | formic (4.39); fumaric (2.24); |
| 0.39 | 2.50 | formic + acetic + propionic + benzoic (1.42); |
| 0.39 | 1.56 | formic (3.92); fumaric (1.58); succinic (3.03); |
| 0.39 | 1.25 | benzoic (1.81); sorbic (2.17); formic + acetic + propionic + benzoic (7.24); |
| 0.39 | 0.78 | fumaric (1.67); |
| 0.39 | 0.63 | benzoic (5.57); sorbic (2.86); |
| 0.39 | 0.39 | fumaric (0.63); |
| 0.39 | 0.31 | benzoic (4.87); sorbic (1.9); |
| 0.39 | 0.16 | sorbic (1.09); |
| 0.20 | 200.00 | calcium formate (12.51); |
| 0.20 | 100.00 | acetic (0.05); butyric (0.04); formic + acetic + propionic (0.68); succinic (0.1); |
| 0.20 | 80.00 | formic + acetic + propionic + benzoic (0.52); |
| 0.20 | 50.00 | acetic (0.03); formic + acetic + propionic (0.8); formic (0.11); |
| 0.20 | 40.00 | formic + acetic + propionic + benzoic (0.97); |
| 0.20 | 25.00 | formic + acetic + propionic (0.79); succinic (0.61); |
| 0.20 | 20.00 | formic + acetic + propionic + benzoic (31.19); |
| 0.20 | 12.50 | acetic (3.1); formic + acetic + propionic (19.81); fumaric (7.87); propionic (4.82); succinic (7.59); |
| 0.20 | 10.00 | sorbic (3.73); formic + acetic + propionic + benzoic (33.35); |
| 0.20 | 6.25 | formic + acetic + propionic (7.14); fumaric (4.17); valeric (0.64); |
| 0.20 | 5.00 | formic + acetic + propionic + benzoic (8.17); |
| 0.20 | 3.13 | formic + acetic + propionic (1.06); formic (5.07); succinic (1.63); |
| 0.20 | 2.50 | sorbic (4.2); formic + acetic + propionic + benzoic (2.7); |
| 0.20 | 1.56 | formic + acetic + propionic (2.42); formic (3.9); fumaric (0.56); butyric (4.11); |
| 0.20 | 1.25 | sorbic (1.9); formic + acetic + propionic + benzoic (8.39); |
| 0.20 | 0.63 | benzoic (4.64); sorbic (2.02); |
| 0.20 | 0.31 | benzoic (2.39); sorbic (1.64); |
| 0.20 | 0.16 | sorbic (1.45); |

TABLE 6
Synergistic inhibition of microbial growth of Salmonella enterica subsp. enterica serotype typhimurium SL1344 (DSMZ
24522) upon treatment with different concentrations of SHP in combination with different concentrations of organic
acid(s) or salt(s) thereof. In the details column, the tested organic acid(s) or salt(s) thereof is indicated followed by
the observed “S” value in brackets. Individual experimental results are separated by semicolon(s).

| SHP | acid/salt | |
|---|---|---|
| mM | mM | details |
| 100.00 | 100.00 | octanoic (0); valeric (0.02); |
| 100.00 | 50.00 | butyric (0.05); octanoic (0.41); pyruvic (0.26); |
| 100.00 | 25.00 | butyric (0.32); lactic (1.4); octanoic (1.25); pyruvic (0.95); valeric (0.06); |
| 100.00 | 20.00 | formic + acetic + propionic + benzoic (15.77); |
| 100.00 | 12.50 | formic + acetic + propionic (15.81); citric (0.12); fumaric (5.73); octanoic (9.16); valeric (2.23); |
| 100.00 | 10.00 | formic + acetic + propionic + benzoic (12.02); |
| 100.00 | 6.25 | formic (4); fumaric (1.07); lactic (5.31); sodium benzoate (7.18); |
| 100.00 | 5.00 | sorbic (2.55); formic + acetic + propionic + benzoic (2.74); |
| 100.00 | 3.13 | acetic (1.93); formic (1.01); fumaric (4.15); lactic (3.9); propionic (2.42); valeric (3.53); |
| 100.00 | 1.56 | acetic (3.44); formic (5.15); fumaric (0.62); lactic (4.22); pyruvic (0.96); valeric (7.12); |
| 100.00 | 0.78 | fumaric (1.06); |
| 100.00 | 0.16 | sorbic (4.62); |
| 50.00 | 100.00 | butyric (0.31); pyruvic (0); |
| 50.00 | 50.00 | octanoic (1.21); pyruvic (0.05); |
| 50.00 | 25.00 | butyric (0.48); lactic (1.49); octanoic (2.71); pyruvic (0.8); |
| 50.00 | 20.00 | formic + acetic + propionic + benzoic (13.28); |
| 50.00 | 12.50 | butyric (0.01); formic + acetic + propionic (15.9); fumaric (4.31); octanoic (10.59); valeric (2.71); |
| 50.00 | 10.00 | benzoic (1.23); sorbic (8.02); formic + acetic + propionic + benzoic (5.89); |
| 50.00 | 6.25 | formic (0.37); lactic (2.66); sodium benzoate (8.29); valeric (11.11); |
| 50.00 | 5.00 | benzoic (4.16); sorbic (21.72); |
| 50.00 | 3.13 | fumaric (0.98); propionic (0.15); |
| 50.00 | 2.50 | sorbic (1.13); |
| 50.00 | 1.56 | acetic (1.95); formic (1.84); valeric (9.79); |
| 50.00 | 1.25 | sorbic (0.34); |
| 50.00 | 0.78 | fumaric (0.58); |
| 50.00 | 0.39 | fumaric (6.2); |
| 50.00 | 0.31 | sorbic (11.65); |
| 50.00 | 0.16 | sorbic (10.55); |
| 25.00 | 100.00 | pyruvic (0.08); |
| 25.00 | 50.00 | butyric (0.03); pyruvic (0.17); |
| 25.00 | 25.00 | lactic (0.43); octanoic (1.56); pyruvic (0.9); valeric (0.02); |
| 25.00 | 20.00 | formic + acetic + propionic + benzoic (14.28); |
| 25.00 | 12.50 | formic + acetic + propionic (14.22); fumaric (0.67); octanoic (6.64); valeric (2.78); |
| 25.00 | 10.00 | formic + acetic + propionic + benzoic (5.96); |
| 25.00 | 6.25 | calcium formate (2.78); sodium benzoate (16.76); octanoic (4.16); valeric (13.91); |
| 25.00 | 5.00 | sorbic (28.51); |
| 25.00 | 3.13 | propionic (1.14); valeric (3.47); |
| 25.00 | 2.50 | sorbic (5.04); |
| 25.00 | 1.56 | acetic (4.67); formic (0.1); lactic (0.68); valeric (14.59); |
| 25.00 | 1.25 | sorbic (4.89); |
| 25.00 | 0.63 | sorbic (13.54); |
| 25.00 | 0.31 | sorbic (25.64); |
| 25.00 | 0.16 | sorbic (24.95); |
| 12.50 | 200.00 | calcium formate (2.57); |
| 12.50 | 100.00 | calcium formate (4.79); octanoic (0.93); valeric (0.01); |
| 12.50 | 50.00 | calcium formate (6.79); octanoic (2.58); pyruvic (0.22); |
| 12.50 | 25.00 | lactic (0.49); octanoic (2.39); pyruvic (0.85); valeric (0.02); |
| 12.50 | 20.00 | formic + acetic + propionic + benzoic (16.74); |
| 12.50 | 12.50 | formic + acetic + propionic (9.66); fumaric (4.41); sodium benzoate (9.94); propionic (0.12); valeric (3.05); |
| 12.50 | 10.00 | formic + acetic + propionic + benzoic (4.07); |
| 12.50 | 6.25 | calcium formate (16.84); fumaric (7.51); sodium benzoate (31.08); octanoic (1.79); valeric (14.92); |
| 12.50 | 5.00 | benzoic (1.13); sorbic (15.47); |
| 12.50 | 3.13 | fumaric (2.71); valeric (4.67); |
| 12.50 | 1.56 | acetic (4.91); formic (1.57); fumaric (4.69); valeric (17.58); |
| 12.50 | 0.78 | fumaric (6.91); |
| 12.50 | 0.39 | fumaric (9.99); |
| 12.50 | 0.31 | sorbic (13.94); |
| 12.50 | 0.16 | sorbic (23.1); |
| 6.25 | 200.00 | calcium formate (9.36); |
| 6.25 | 100.00 | butyric (0.06); calcium formate (3.69); sodium benzoate (9.78); octanoic (3.42); pyruvic (0.01); |
| 6.25 | 50.00 | butyric (0.02); calcium formate (7.19); sodium benzoate (15.86); octanoic (5.11); |
| 6.25 | 25.00 | lactic (1); octanoic (3.58); pyruvic (0.57); |
| 6.25 | 20.00 | formic + acetic + propionic + benzoic (47.47); |
| 6.25 | 12.50 | formic + acetic + propionic (13.06); formic (0.27); fumaric (0.35); lactic (5.98); sodium benzoate (12.26); |
| | | octanoic (4.66); valeric (3.18); |
| 6.25 | 10.00 | benzoic (0.92); sorbic (25.99); formic + acetic + propionic + benzoic (36.27); |
| 6.25 | 6.25 | calcium formate (14.2); sodium benzoate (41.63); octanoic (11.04); valeric (16.85); |
| 6.25 | 5.00 | sorbic (29.36); |
| 6.25 | 3.13 | citric (5.78); octanoic (1.24); valeric (9.06); |
| 6.25 | 2.50 | sorbic (3.18); |
| 6.25 | 1.56 | formic (0.08); octanoic (4.75); valeric (9.63); |
| 6.25 | 1.25 | sorbic (5.2); |
| 6.25 | 0.63 | sorbic (9.93); |
| 6.25 | 0.39 | fumaric (1.13); |
| 6.25 | 0.31 | sorbic (26.98); |
| 6.25 | 0.16 | sorbic (34.8); |
| 3.13 | 200.00 | calcium formate (10.46); |
| 3.13 | 100.00 | calcium formate (1.32); sodium benzoate (2.68); octanoic (8.01); |
| 3.13 | 50.00 | calcium formate (7.19); sodium benzoate (4.93); octanoic (10.57); |
| 3.13 | 25.00 | octanoic (8.9); pyruvic (0.74); |
| 3.13 | 20.00 | formic + acetic + propionic + benzoic (53); |
| 3.13 | 12.50 | formic + acetic + propionic (0.86); citric (0.45); formic (1.09); fumaric (5.26); lactic (16.98); octanoic (14.66); |
| 3.13 | 10.00 | formic + acetic + propionic + benzoic (58.94); |
| 3.13 | 6.25 | calcium formate (9.86); citric (2.85); fumaric (7.12); octanoic (21.56); pyruvic (7.77); valeric (4.12); |
| 3.13 | 5.00 | sorbic (27.72); |
| 3.13 | 3.13 | citric (22.06); fumaric (2.11); octanoic (11.99); pyruvic (0.55); |
| 3.13 | 1.56 | citric (11.59); octanoic (14.92); |
| 3.13 | 1.25 | sorbic (1.21); |
| 3.13 | 0.63 | sorbic (3.88); |
| 3.13 | 0.39 | fumaric (0.31); |
| 3.13 | 0.31 | sorbic (17.34); |
| 3.13 | 0.16 | sorbic (28.66); |
| 1.56 | 200.00 | calcium formate (12.83); |
| 1.56 | 100.00 | calcium formate (4.36); sodium benzoate (2.8); octanoic (7.13); |
| 1.56 | 50.00 | calcium formate (9.99); octanoic (9.88); |
| 1.56 | 25.00 | calcium formate (4.63); lactic (2.46); octanoic (9.02); pyruvic (0.39); |
| 1.56 | 20.00 | formic + acetic + propionic + benzoic (54.44); |
| 1.56 | 12.50 | formic + acetic + propionic (5.17); citric (1.52); formic (1.64); fumaric (4); lactic (33.82); octanoic (16.01); |
| | | pyruvic (4.86); valeric (3.23); |
| 1.56 | 10.00 | benzoic (2.46); sorbic (15.95); formic + acetic + propionic + benzoic (44.74); |
| 1.56 | 6.25 | calcium formate (4.5); citric (3.63); fumaric (4.45); lactic (11.36); octanoic (9.33); pyruvic (17.2); valeric |
| | | (17.6); |
| 1.56 | 5.00 | benzoic (6.8); sorbic (13.38); |
| 1.56 | 3.13 | formic + acetic + propionic (0.86); citric (28.76); fumaric (6.71); lactic (3.63); octanoic (15.21); pyruvic (0.66); |
| | | valeric (8.79); |
| 1.56 | 1.56 | citric (9.04); lactic (2.88); octanoic (14.76); pyruvic (2.2); valeric (8.52); |
| 1.56 | 0.39 | fumaric (0.94); |
| 1.56 | 0.16 | sorbic (2.9); |
| 0.78 | 200.00 | calcium formate (10.18); |
| 0.78 | 100.00 | calcium formate (3.28); octanoic (4.56); |
| 0.78 | 80.00 | formic + acetic + propionic + benzoic (0.21); |
| 0.78 | 50.00 | calcium formate (10.14); octanoic (7.04); |
| 0.78 | 40.00 | formic + acetic + propionic + benzoic (0.02); |
| 0.78 | 25.00 | calcium formate (3.09); lactic (2.7); octanoic (1.1); |
| 0.78 | 20.00 | formic + acetic + propionic + benzoic (69); |
| 0.78 | 12.50 | formic + acetic + propionic (6.76); calcium formate (2.54); citric (2.58); formic (2.58); fumaric (8.79); lactic |
| | | (40.84); octanoic (9.44); propionic (0.08); pyruvic (9.37); succinic (1.85); valeric (3.99); |
| 0.78 | 10.00 | formic + acetic + propionic + benzoic (57.51); |
| 0.78 | 6.25 | acetic (7.4); calcium formate (10.59); citric (6.9); fumaric (14.56); lactic (0.67); pyruvic (15.23); valeric |
| | | (8.85); |
| 0.78 | 5.00 | benzoic (12.79); sorbic (16.78); formic + acetic + propionic + benzoic (6.02); |
| 0.78 | 3.13 | citric (37.03); fumaric (9.13); lactic (1.84); octanoic (9.48); pyruvic (1.7); valeric (13.85); |
| 0.78 | 2.50 | benzoic (3.85); |
| 0.78 | 1.56 | citric (10.07); lactic (5.37); pyruvic (3.34); valeric (1.18); |
| 0.78 | 0.39 | fumaric (0.85); |
| 0.39 | 200.00 | calcium formate (8.97); |
| 0.39 | 100.00 | calcium formate (9.03); formic (0.2); octanoic (5.23); pyruvic (0.02); |
| 0.39 | 80.00 | formic + acetic + propionic + benzoic (0.85); |
| 0.39 | 50.00 | calcium formate (10.56); octanoic (10.2); |
| 0.39 | 40.00 | formic + acetic + propionic + benzoic (0.48); |
| 0.39 | 25.00 | calcium formate (3.6); lactic (1.8); octanoic (11.65); valeric (0.03); |
| 0.39 | 20.00 | formic + acetic + propionic + benzoic (72.65); |
| 0.39 | 12.50 | acetic (0.89); butyric (0.4); formic + acetic + propionic + benzoic (36.54); calcium formate (7.28); citric (5.45); |
| | | formic (3.67); fumaric (17.37); lactic (46.53); octanoic (9.23); propionic (0.44); pyruvic (9.76); succinic |
| | | (10.17); valeric (3.33); |
| 0.39 | 10.00 | benzoic (3.22); sorbic (15.23); |
| 0.39 | 6.25 | acetic (15.1); calcium formate (10.03); citric (13.37); fumaric (33.93); lactic (13.18); pyruvic (12.74); |
| | | valeric (14.84); |
| 0.39 | 5.00 | formic + acetic + propionic + benzoic (8.69); |
| 0.39 | 3.13 | citric (47.96); fumaric (36.02); lactic (7.2); pyruvic (7.04); succinic (6.61); |
| 0.39 | 2.50 | benzoic (18.5); sorbic (3.54); formic + acetic + propionic + benzoic (2.95); |
| 0.39 | 1.56 | citric (20.44); formic (3.9); fumaric (27.76); lactic (7.17); octanoic (3.87); pyruvic (7.43); succinic (13.18); |
| 0.39 | 1.25 | sorbic (3.02); formic + acetic + propionic + benzoic (1.32); |
| 0.39 | 0.78 | fumaric (15.08); |
| 0.39 | 0.39 | fumaric (20.49); |
| 0.39 | 0.31 | sorbic (7.57); |
| 0.39 | 0.16 | sorbic (6.63); |
| 0.20 | 200.00 | calcium formate (8.12); |
| 0.20 | 100.00 | acetic (0.07); butyric (0.31); calcium formate (3.2); formic (0.13); octanoic (4.67); |
| 0.20 | 80.00 | formic + acetic + propionic + benzoic (0.05); |
| 0.20 | 50.00 | acetic (0.13); calcium formate (5.94); octanoic (8.03); |
| 0.20 | 40.00 | formic + acetic + propionic + benzoic (0.66); |
| 0.20 | 25.00 | acetic (0.22); calcium formate (0.93); lactic (5.04); octanoic (11.16); pyruvic (0.47); valeric (0.07); |
| 0.20 | 20.00 | formic + acetic + propionic + benzoic (63.93); |
| 0.20 | 12.50 | acetic (4.36); butyric (5.38); calcium formate (3.52); fumaric (16.5); lactic (42.06); propionic (4.2); pyruvic |
| | | (16.01); succinic (6.29); |
| 0.20 | 10.00 | sorbic (1.77); formic + acetic + propionic + benzoic (13.25); |
| 0.20 | 6.25 | acetic (22.39); butyric (11.37); calcium formate (15.85); citric (16.51); formic (5.7); fumaric (24.03); lactic |
| | | (27.11); propionic (11.12); pyruvic (12.76); |
| 0.20 | 5.00 | benzoic (14.94); sorbic (11.46); formic + acetic + propionic + benzoic (9.39); |
| 0.20 | 3.13 | acetic (2.19); citric (45.22); formic (6.67); fumaric (35.71); lactic (15.99); pyruvic (12.59); succinic (0.71); |
| 0.20 | 2.50 | benzoic (1.72); formic + acetic + propionic + benzoic (7.14); |
| 0.20 | 1.56 | citric (27.34); formic (13.41); fumaric (1.29); lactic (8.32); |
| 0.20 | 1.25 | formic + acetic + propionic + benzoic (6.96); |
| 0.20 | 0.78 | fumaric (0.87); |
| 0.20 | 0.39 | fumaric (5.87); |
| 0.20 | 0.16 | sorbic (5.31); |

TABLE 7
Synergistic inhibition of microbial growth of Salmonella enterica subsp. enterica serotype typhimurium SL1344 (DSMZ
24522) upon treatment with different concentrations of MHP in combination with different concentrations of organic
acid(s) or salt(s) thereof. In the details column, the tested organic acid(s) or salt(s) thereof is indicated followed by
the observed “S” value in brackets. Individual experimental results are separated by semicolon(s).

| MHP | acid/salt | |
|---|---|---|
| mM | mM | details |
| 100.00 | 50.00 | calcium formate (1.95); sodium benzoate (6.75); |
| 100.00 | 25.00 | sodium benzoate (2.11); |
| 100.00 | 20.00 | formic + acetic + propionic + benzoic (7.68); |
| 100.00 | 12.50 | formic+acetic+propionic (12.01); calcium formate (9.39); |
| 100.00 | 10.00 | formic + acetic + propionic + benzoic (16.29); |
| 100.00 | 6.25 | formic + acetic + propionic (7.2); calcium formate (5.64); formic (1.8); |
| 100.00 | 5.00 | formic + acetic + propionic + benzoic (7.63); |
| 100.00 | 3.13 | acetic (8.19); |
| 100.00 | 1.56 | acetic (6.9); formic (3.45); |
| 100.00 | 1.25 | benzoic (7.4); |
| 100.00 | 0.63 | benzoic (7.34); |
| 100.00 | 0.31 | benzoic (8.39); |
| 50.00 | 50.00 | sodium benzoate (1.4); |
| 50.00 | 20.00 | formic + acetic + propionic + benzoic (5.9); |
| 50.00 | 12.50 | formic + acetic + propionic (13.74); calcium formate (4.98); |
| 50.00 | 10.00 | formic + acetic + propionic + benzoic (8.88); |
| 50.00 | 6.25 | formic + acetic + propionic (7.37); calcium formate (4.42); formic (0.64); |
| 50.00 | 5.00 | formic + acetic + propionic + benzoic (4.26); |
| 50.00 | 3.13 | acetic (4.74); formic (1.24); propionic (5.36); |
| 50.00 | 1.56 | acetic (7.5); formic (5.07); propionic (7.49); |
| 50.00 | 1.25 | benzoic (2.43); |
| 50.00 | 0.63 | benzoic (0); |
| 50.00 | 0.31 | benzoic (1.77); |
| 25.00 | 100.00 | acetic (0.04); |
| 25.00 | 50.00 | sodium benzoate (1.79); |
| 25.00 | 25.00 | lactic (0.05); sodium benzoate (0.43); |
| 25.00 | 20.00 | formic + acetic + propionic + benzoic (5.25); |
| 25.00 | 12.50 | formic + acetic + propionic (9.04); formic + acetic + propionic + benzoic (7.29); |
| 25.00 | 6.25 | acetic (4.24); formic + acetic + propionic (1.09); calcium formate (10.36); formic (2.29); |
| 25.00 | 3.13 | acetic (13.56); formic (3.97); propionic (1.29); |
| 25.00 | 1.56 | acetic (11.42); formic (6.45); propionic (4.24); |
| 12.50 | 200.00 | calcium formate (18.67); |
| 12.50 | 25.00 | lactic (0.18); |
| 12.50 | 20.00 | formic + acetic + propionic + benzoic (7.69); |
| 12.50 | 12.50 | formic + acetic + propionic (6.59); calcium formate (22.04); |
| 12.50 | 10.00 | formic + acetic + propionic + benzoic (11.63); |
| 12.50 | 6.25 | calcium formate (8.65); |
| 12.50 | 5.00 | formic + acetic + propionic + benzoic (1.43); |
| 12.50 | 3.13 | acetic (6.2); formic (4.5); lactic (9); propionic (5.18); |
| 12.50 | 1.56 | acetic (6.55); formic (4.74); lactic (7.58); propionic (4.42); |
| 12.50 | 1.25 | benzoic (3.5); |
| 12.50 | 0.63 | benzoic (8.74); |
| 12.50 | 0.31 | benzoic (9.54); |
| 6.25 | 200.00 | calcium formate (25.8); |
| 6.25 | 100.00 | calcium formate (4.02); |
| 6.25 | 50.00 | calcium formate (3.17); |
| 6.25 | 20.00 | formic + acetic + propionic + benzoic (9.35); |
| 6.25 | 12.50 | formic + acetic + propionic (8.9); |
| 6.25 | 10.00 | formic + acetic + propionic + benzoic (21.24); |
| 6.25 | 6.25 | calcium formate (3.56); |
| 6.25 | 3.13 | propionic (2.79); |
| 6.25 | 1.56 | acetic (3.57); formic (0.23); lactic (1.89); propionic (4.81); |
| 6.25 | 0.31 | benzoic (2.56); |
| 3.13 | 200.00 | calcium formate (29.24); |
| 3.13 | 100.00 | acetic (0.02); calcium formate (3.68); |
| 3.13 | 25.00 | lactic (0.47); |
| 3.13 | 20.00 | formic + acetic + propionic + benzoic (18.36); |
| 3.13 | 10.00 | formic + acetic + propionic + benzoic (43.93); |
| 3.13 | 6.25 | lactic (4.3); |
| 3.13 | 5.00 | benzoic (0.33); |
| 3.13 | 3.13 | acetic (15.81); formic (10.76); lactic (17.78); propionic (6.41); |
| 3.13 | 1.56 | acetic (4.42); lactic (5.39); propionic (2.42); |
| 3.13 | 0.63 | benzoic (13.45); |
| 3.13 | 0.31 | benzoic (0.78); |
| 1.56 | 200.00 | calcium formate (36.07); |
| 1.56 | 100.00 | acetic (0.02); calcium formate (7.09); |
| 1.56 | 50.00 | calcium formate (2.11); |
| 1.56 | 25.00 | calcium formate (1.19); |
| 1.56 | 20.00 | formic + acetic + propionic + benzoic (18.02); |
| 1.56 | 12.50 | calcium formate (9.79); |
| 1.56 | 10.00 | formic + acetic + propionic + benzoic (39.28); |
| 1.56 | 5.00 | benzoic (0.81); |
| 1.56 | 3.13 | acetic (1.31); |
| 1.56 | 1.56 | acetic (3.1); |
| 0.78 | 400.00 | calcium formate (0.67); |
| 0.78 | 200.00 | calcium formate (40.84); |
| 0.78 | 100.00 | calcium formate (8.85); |
| 0.78 | 50.00 | calcium formate (10.15); |
| 0.78 | 25.00 | calcium formate (6.4); lactic (0.74); |
| 0.78 | 20.00 | formic + acetic + propionic + benzoic (25.82); |
| 0.78 | 12.50 | calcium formate (27.42); |
| 0.78 | 10.00 | formic + acetic + propionic + benzoic (22.61); |
| 0.78 | 6.25 | lactic (11.39); |
| 0.78 | 5.00 | benzoic (0.04); |
| 0.78 | 3.13 | acetic (6); lactic (18.46); |
| 0.78 | 1.56 | acetic (18.7); citric (3.05); formic (9.49); lactic (30.79); propionic (13.71); |
| 0.78 | 1.25 | benzoic (3.73); |
| 0.78 | 0.63 | benzoic (11.51); |
| 0.78 | 0.31 | benzoic (25.87); |
| 0.39 | 200.00 | calcium formate (39.63); |
| 0.39 | 100.00 | calcium formate (9.03); |
| 0.39 | 50.00 | calcium formate (11.39); |
| 0.39 | 25.00 | calcium formate (2.23); lactic (1.26); |
| 0.39 | 20.00 | formic + acetic + propionic + benzoic (32.44); |
| 0.39 | 12.50 | formic + acetic + propionic (0.54); calcium formate (34.92); |
| 0.39 | 10.00 | formic + acetic + propionic + benzoic (36.11); |
| 0.39 | 6.25 | lactic (11.46); |
| 0.39 | 5.00 | formic + acetic + propionic + benzoic (2.02); |
| 0.39 | 1.25 | benzoic (10.41); |
| 0.39 | 0.63 | benzoic (3.75); |
| 0.39 | 0.31 | benzoic (3.13); |
| 0.20 | 200.00 | calcium formate (37.5); |
| 0.20 | 100.00 | calcium formate (3.11); lactic (0.29); |
| 0.20 | 50.00 | acetic (0.04); calcium formate (12.65); lactic (0.09); |
| 0.20 | 25.00 | calcium formate (6); lactic (1.49); |
| 0.20 | 20.00 | formic + acetic + propionic + benzoic (37.41); |
| 0.20 | 12.50 | acetic (1.31); formic + acetic + propionic (12.87); calcium formate (41.12); |
| 0.20 | 10.00 | formic + acetic + propionic + benzoic (29.72); |
| 0.20 | 6.25 | lactic (28.24); |
| 0.20 | 5.00 | benzoic (2.7); formic + acetic + propionic + benzoic (5.06); |
| 0.20 | 3.13 | acetic (3.93); lactic (6.47); |
| 0.20 | 1.56 | lactic (0.91); |
| 0.20 | 1.25 | benzoic (9.87); |
| 0.20 | 0.63 | benzoic (0.58); |

TABLE 8
Synergistic inhibition of microbial growth of Salmonella enterica subsp. enterica serotype typhimurium SL1344
(DSMZ 24522) upon treatment with different concentrations of PHP in combination with different concentrations
of organic acid(s) or salt(s) thereof. In the details column, the tested organic acid(s) or salt(s) thereof is indicated
followed by the observed “S” value in brackets. Individual experimental results are separated by semicolon(s).

| PHP | acid/salt | |
|---|---|---|
| mM | mM | details |
| 100.00 | 100.00 | octanoic (18.22); valeric (0.08); |
| 100.00 | 50.00 | butyric (0.03); octanoic (17.68); |
| 100.00 | 25.00 | butyric (0.03); octanoic (18.45); succinic (0.16); valeric (0); |
| 100.00 | 12.50 | formic + acetic + propionic (13); formic (4.04); fumaric (8.16); octanoic (19.55); propionic (1.36); succinic |
| | | (1.92); |
| 100.00 | 10.00 | formic + acetic + propionic + benzoic (13.88); |
| 100.00 | 6.25 | formic + acetic + propionic (9.01); formic (2.14); fumaric (19.89); lactic (15.02); sodium benzoate (2.86); |
| | | octanoic (19.99); propionic (0.2); |
| 100.00 | 5.00 | sorbic (18.8); formic + acetic + propionic + benzoic (3.69); |
| 100.00 | 3.13 | formic + acetic + propionic (5.7); fumaric (29.08); lactic (10.5); octanoic (19.24); succinic (17.75); |
| 100.00 | 2.50 | benzoic (29.76); sorbic (36.28); |
| 100.00 | 1.56 | formic + acetic + propionic (5.54); formic (0.57); fumaric (27.6); lactic (2.26); octanoic (20.84); succinic |
| | | (19.27); |
| 100.00 | 1.25 | benzoic (18.82); sorbic (38.52); formic + acetic + propionic + benzoic (1.81); |
| 100.00 | 0.78 | fumaric (25.19); |
| 100.00 | 0.63 | benzoic (19.42); sorbic (35.11); |
| 100.00 | 0.39 | fumaric (33.71); |
| 100.00 | 0.31 | benzoic (5.46); sorbic (35.1); |
| 100.00 | 0.16 | sorbic (35.91); |
| 50.00 | 100.00 | butyric (0.01); octanoic (15.31); |
| 50.00 | 50.00 | octanoic (14.48); |
| 50.00 | 25.00 | butyric (0.08); fumaric (0.01); octanoic (15.14); succinic (0.01); |
| 50.00 | 12.50 | acetic (0.8); formic + acetic + propionic (7.12); formic (4.74); fumaric (7.71); octanoic (15.78); propionic |
| | | (1.73); valeric (0.02); |
| 50.00 | 10.00 | sorbic (5.34); formic + acetic + propionic + benzoic (2.19); |
| 50.00 | 6.25 | formic + acetic + propionic (4.11); formic (2.3); fumaric (20.1); sodium benzoate (6.03); octanoic (14.28); |
| 50.00 | 5.00 | sorbic (15.19); |
| 50.00 | 3.13 | formic + acetic + propionic (1.94); fumaric (30.57); octanoic (12.33); succinic (10.4); |
| 50.00 | 2.50 | benzoic (14.65); sorbic (25.51); |
| 50.00 | 1.56 | acetic (1.27); formic + acetic + propionic (1.76); fumaric (28.05); lactic (3.75); octanoic (14.56); succinic |
| | | (13.21); |
| 50.00 | 1.25 | benzoic (0.63); sorbic (26.04); |
| 50.00 | 0.78 | fumaric (25.94); |
| 50.00 | 0.63 | sorbic (25.19); |
| 50.00 | 0.39 | fumaric (34.71); |
| 50.00 | 0.31 | sorbic (23.31); |
| 50.00 | 0.16 | sorbic (23.84); |
| 25.00 | 400.00 | calcium formate (7.37); |
| 25.00 | 100.00 | calcium formate (21.2); octanoic (10.38); valeric (0.05); |
| 25.00 | 50.00 | calcium formate (13.49); octanoic (9.99); valeric (0.04); |
| 25.00 | 25.00 | calcium formate (4.01); fumaric (0.12); octanoic (9.77); valeric (0); |
| 25.00 | 12.50 | formic + acetic + propionic (3.43); calcium formate (8.99); formic (0.42); fumaric (9.89); sodium benzoate |
| | | (13.14); octanoic (8.45); propionic (1.25); |
| 25.00 | 10.00 | sorbic (6.12); formic + acetic + propionic + benzoic (1.77); |
| 25.00 | 6.25 | calcium formate (17.11); fumaric (28.08); sodium benzoate (25.38); octanoic (5.79); succinic (4.17); |
| 25.00 | 5.00 | sorbic (18.17); |
| 25.00 | 3.13 | fumaric (37.99); octanoic (4.73); succinic (18.27); |
| 25.00 | 2.50 | benzoic (18.18); sorbic (29.25); |
| 25.00 | 1.56 | fumaric (35.48); octanoic (4.92); succinic (19.46); |
| 25.00 | 1.25 | benzoic (5.76); sorbic (26.93); |
| 25.00 | 0.78 | fumaric (33.07); |
| 25.00 | 0.63 | sorbic (26.57); |
| 25.00 | 0.39 | fumaric (42.36); |
| 25.00 | 0.31 | sorbic (26.41); |
| 25.00 | 0.16 | sorbic (28.7); |
| 12.50 | 400.00 | calcium formate (7.68); |
| 12.50 | 100.00 | calcium formate (29.46); sodium benzoate (2.04); octanoic (8.92); |
| 12.50 | 50.00 | calcium formate (19.47); sodium benzoate (2.67); octanoic (8.46); valeric (0.04); |
| 12.50 | 25.00 | calcium formate (12.54); sodium benzoate (7.58); octanoic (8.1); |
| 12.50 | 12.50 | acetic (0.31); formic + acetic + propionic (0.37); formic (3.33); fumaric (8.17); sodium benzoate (25.45); |
| | | octanoic (1.93); propionic (1.8); |
| 12.50 | 10.00 | formic + acetic + propionic + benzoic (20.74); |
| 12.50 | 6.25 | calcium formate (14.05); formic (0.88); fumaric (21.85); sodium benzoate (43.98); succinic (4.77); |
| 12.50 | 5.00 | sorbic (22.84); |
| 12.50 | 3.13 | fumaric (28.19); succinic (10.05); |
| 12.50 | 2.50 | benzoic (19.31); sorbic (32.35); |
| 12.50 | 1.56 | formic (1.33); fumaric (28.48); octanoic (0.41); succinic (12.59); |
| 12.50 | 1.25 | benzoic (3.63); sorbic (27.92); |
| 12.50 | 0.78 | fumaric (26.71); |
| 12.50 | 0.63 | sorbic (33.61); |
| 12.50 | 0.39 | fumaric (36.38); |
| 12.50 | 0.31 | sorbic (35.3); |
| 12.50 | 0.16 | sorbic (35.94); |
| 6.25 | 400.00 | calcium formate (1.98); |
| 6.25 | 200.00 | calcium formate (1.69); |
| 6.25 | 100.00 | calcium formate (22.27); sodium benzoate (13.58); octanoic (18.29); |
| 6.25 | 50.00 | calcium formate (17.3); sodium benzoate (21.37); octanoic (17.96); |
| 6.25 | 25.00 | calcium formate (10.99); fumaric (0.01); sodium benzoate (11.19); octanoic (18.23); |
| 6.25 | 12.50 | formic (2.72); fumaric (7.49); sodium benzoate (28.83); octanoic (11.99); propionic (2.05); valeric (0.06); |
| 6.25 | 10.00 | sorbic (6.44); formic + acetic + propionic + benzoic (56.86); |
| 6.25 | 6.25 | calcium formate (16.14); fumaric (19.74); sodium benzoate (48.79); octanoic (10.17); propionic (1.2); |
| | | succinic (6); valeric (0.64); |
| 6.25 | 5.00 | sorbic (18.95); |
| 6.25 | 3.13 | fumaric (28.54); octanoic (2.22); succinic (13.13); |
| 6.25 | 2.50 | benzoic (16.2); sorbic (26.95); |
| 6.25 | 1.56 | formic (1.73); fumaric (25.27); octanoic (11.28); propionic (1.31); succinic (10.86); |
| 6.25 | 1.25 | sorbic (18.45); |
| 6.25 | 0.78 | fumaric (23.72); |
| 6.25 | 0.63 | sorbic (21.11); |
| 6.25 | 0.39 | fumaric (33.29); |
| 6.25 | 0.31 | sorbic (27.33); |
| 6.25 | 0.16 | sorbic (27.45); |
| 3.13 | 400.00 | calcium formate (0.45); |
| 3.13 | 100.00 | calcium formate (21.77); sodium benzoate (13.63); octanoic (18.21); valeric (0); |
| 3.13 | 50.00 | calcium formate (18.53); sodium benzoate (22.11); octanoic (17.75); |
| 3.13 | 25.00 | calcium formate (6.73); sodium benzoate (11.59); octanoic (17.2); |
| 3.13 | 12.50 | formic (0.09); fumaric (4.2); sodium benzoate (26.49); octanoic (12.1); propionic (1.33); |
| 3.13 | 10.00 | sorbic (4.97); formic + acetic + propionic + benzoic (66.7); |
| 3.13 | 6.25 | calcium formate (10.41); fumaric (11.64); sodium benzoate (43.01); octanoic (3.87); succinic (0.15); |
| 3.13 | 5.00 | sorbic (15.52); |
| 3.13 | 3.13 | fumaric (15.99); octanoic (1.44); succinic (1.09); |
| 3.13 | 2.50 | benzoic (16.93); sorbic (18.78); |
| 3.13 | 1.56 | fumaric (13.37); lactic (1.56); octanoic (1.76); succinic (0.43); |
| 3.13 | 1.25 | benzoic (2.68); sorbic (1.2); |
| 3.13 | 0.78 | fumaric (13.22); |
| 3.13 | 0.63 | sorbic (2.75); |
| 3.13 | 0.39 | fumaric (18.62); |
| 3.13 | 0.31 | sorbic (11.01); |
| 3.13 | 0.16 | sorbic (12.46); |
| 1.56 | 100.00 | calcium formate (24.85); sodium benzoate (14.49); octanoic (21.66); valeric (0); |
| 1.56 | 80.00 | formic + acetic + propionic + benzoic (0.58); |
| 1.56 | 50.00 | calcium formate (20.09); sodium benzoate (23.01); octanoic (20.83); |
| 1.56 | 25.00 | calcium formate (12.91); sodium benzoate (10.18); octanoic (19.53); succinic (0.28); |
| 1.56 | 12.50 | calcium formate (0.4); formic (1.64); fumaric (5.08); sodium benzoate (25.1); octanoic (10.48); propionic |
| | | (1.6); succinic (1.63); |
| 1.56 | 10.00 | sorbic (5.04); formic + acetic + propionic + benzoic (70.66); |
| 1.56 | 6.25 | calcium formate (16.91); fumaric (15.3); sodium benzoate (46.99); succinic (13.59); |
| 1.56 | 5.00 | sorbic (14.44); formic + acetic + propionic + benzoic (4.75); |
| 1.56 | 3.13 | fumaric (19.26); lactic (8.01); succinic (19.92); |
| 1.56 | 2.50 | benzoic (16.68); sorbic (11.66); |
| 1.56 | 1.56 | fumaric (17.15); lactic (9.29); octanoic (0.27); succinic (17.07); |
| 1.56 | 0.78 | fumaric (15.34); |
| 1.56 | 0.39 | fumaric (20.21); |
| 1.56 | 0.16 | sorbic (2.96); |
| 0.78 | 100.00 | calcium formate (12.4); sodium benzoate (1.99); octanoic (24.31); |
| 0.78 | 80.00 | formic + acetic + propionic + benzoic (0.21); |
| 0.78 | 50.00 | calcium formate (9.88); sodium benzoate (5.83); octanoic (23.52); |
| 0.78 | 25.00 | calcium formate (3.58); sodium benzoate (12.99); octanoic (22.02); succinic (0.31); |
| 0.78 | 12.50 | acetic (0.31); formic (10.45); fumaric (4.38); sodium benzoate (35.48); octanoic (10.01); propionic (2.08); |
| | | succinic (3.43); |
| 0.78 | 10.00 | sorbic (5.75); formic + acetic + propionic + benzoic (36.85); |
| 0.78 | 6.25 | calcium formate (3.09); fumaric (13.87); sodium benzoate (55.14); succinic (13.53); valeric (0.03); |
| 0.78 | 5.00 | sorbic (16.89); formic + acetic + propionic + benzoic (7.85); |
| 0.78 | 3.13 | fumaric (15.1); succinic (17.79); |
| 0.78 | 2.50 | benzoic (13.26); formic + acetic + propionic + benzoic (0.34); |
| 0.78 | 1.56 | fumaric (5.53); lactic (0.7); succinic (8.02); |
| 0.78 | 1.25 | formic + acetic + propionic + benzoic (0.97); |
| 0.78 | 0.39 | fumaric (4.17); |
| 0.78 | 0.31 | benzoic (0.38); |
| 0.39 | 200.00 | sodium benzoate (2.08); |
| 0.39 | 100.00 | calcium formate (23.22); formic (0.12); sodium benzoate (6.45); octanoic (30.16); succinic (0.09); |
| 0.39 | 80.00 | formic + acetic + propionic + benzoic (0.58); |
| 0.39 | 50.00 | calcium formate (12.87); sodium benzoate (0.84); octanoic (29.68); |
| 0.39 | 40.00 | formic + acetic + propionic + benzoic (0.01); |
| 0.39 | 25.00 | calcium formate (15.41); sodium benzoate (10.68); octanoic (30.21); succinic (0.35); valeric (0.01); |
| 0.39 | 20.00 | formic + acetic + propionic + benzoic (0.42); |
| 0.39 | 12.50 | acetic (2.45); calcium formate (14.6); formic (2.62); fumaric (7.42); sodium benzoate (32.15); octanoic |
| | | (10.33); propionic (3); succinic (4.95); |
| 0.39 | 10.00 | formic + acetic + propionic + benzoic (26.7); |
| 0.39 | 6.25 | acetic (3.61); calcium formate (11.96); fumaric (22.2); sodium benzoate (47.9); octanoic (2.69); propionic |
| | | (1.15); succinic (21.89); valeric (2.28); |
| 0.39 | 5.00 | sorbic (14.32); formic + acetic + propionic + benzoic (7.46); |
| 0.39 | 3.13 | fumaric (22.32); lactic (3.99); succinic (24.67); |
| 0.39 | 2.50 | formic + acetic + propionic + benzoic (2.61); |
| 0.39 | 1.56 | fumaric (2.19); lactic (3.51); octanoic (0.92); succinic (7.95); |
| 0.39 | 1.25 | formic + acetic + propionic + benzoic (2.62); |
| 0.39 | 0.39 | fumaric (2.74); |
| 0.20 | 200.00 | sodium benzoate (2.56); |
| 0.20 | 100.00 | calcium formate (20.57); formic (0.17); sodium benzoate (1.59); octanoic (36.61); propionic (0.09); |
| | | succinic (0.12); |
| 0.20 | 80.00 | formic + acetic + propionic + benzoic (0.46); |
| 0.20 | 50.00 | calcium formate (7.43); sodium benzoate (1.83); octanoic (35.49); |
| 0.20 | 40.00 | formic + acetic + propionic + benzoic (0.3); |
| 0.20 | 25.00 | calcium formate (16.97); fumaric (0.01); sodium benzoate (6.78); octanoic (35.68); |
| 0.20 | 20.00 | formic + acetic + propionic + benzoic (0.49); |
| 0.20 | 12.50 | acetic (6.73); formic + acetic + propionic (7.73); calcium formate (12.04); fumaric (9.3); sodium benzoate |
| | | (26.85); octanoic (12.79); propionic (7.87); succinic (5.99); valeric (0.34); |
| 0.20 | 10.00 | sorbic (6.68); formic + acetic + propionic + benzoic (6.84); |
| 0.20 | 6.25 | acetic (13.5); butyric (8.01); calcium formate (2.71); fumaric (26.33); lactic (1.7); sodium benzoate |
| | | (16.89); octanoic (7.85); propionic (13.71); succinic (21.82); valeric (22.76); |
| 0.20 | 5.00 | sorbic (18.46); formic + acetic + propionic + benzoic (4.01); |
| 0.20 | 3.13 | acetic (4.77); fumaric (18); lactic (1.97); octanoic (6.93); succinic (19.52); valeric (0.09); |
| 0.20 | 2.50 | benzoic (24.27); sorbic (7.8); formic + acetic + propionic + benzoic (3.51); |
| 0.20 | 1.56 | formic (0.78); fumaric (1.18); lactic (1.12); octanoic (4.06); butyric (4.11); valeric (6.19); |
| 0.20 | 1.25 | sorbic (4.9); formic + acetic + propionic + benzoic (9.9); |
| 0.20 | 0.63 | sorbic (3.53); |
| 0.20 | 0.39 | fumaric (6.01); |
| 0.20 | 0.31 | benzoic (2.74); sorbic (2.77); |
| 0.20 | 0.16 | sorbic (3.17); |

TABLE 9
Synergistic inhibition of microbial growth of Shigella sonnei ATCC 29930,
I virulent, WDCM 00127, CECT 4887 (DSMZ 5570) upon treatment
with different concentrations of SHP in combination with different
concentrations of organic acid(s) or salt(s) thereof. In the details
column, the tested organic acid(s) or salt(s) thereof is indicated
followed by the observed “S” value in brackets. Individual
experimental results are separated by semicolon(s).

| SHP | acid/salt | |
|---|---|---|
| mM | mM | details |
| 100.00 | 100.00 | propionic (0.26); pyruvic (0.47); valeric (0.35); |
| 100.00 | 50.00 | butyric (0.53); pyruvic (1.58); |
| 100.00 | 25.00 | butyric (1.23); lactic (0.17); pyruvic (2); |
| 100.00 | 12.50 | butyric (3.36); citric (3.93); propionic (0.71); |
| | | pyruvic (7.81); succinic (2.7); |
| 100.00 | 6.25 | citric (5.59); propionic (13.75); |
| 100.00 | 5.00 | benzoic (9.8); |
| 100.00 | 3.13 | citric (7.2); lactic (5.55); propionic (2.46); |
| 100.00 | 2.50 | benzoic (0); |
| 100.00 | 1.56 | lactic (2.01); |
| 100.00 | 1.25 | benzoic (5.35); |
| 100.00 | 0.31 | benzoic (4.1); |
| 50.00 | 100.00 | butyric (0.7); propionic (0.35); pyruvic (0.64); |
| 50.00 | 50.00 | pyruvic (0.55); |
| 50.00 | 25.00 | butyric (1.23); pyruvic (1.09); |
| 50.00 | 12.50 | butyric (3.22); citric (3.96); propionic (1.69); |
| 50.00 | 6.25 | citric (2.62); propionic (20.38); |
| 50.00 | 5.00 | benzoic (1.72); |
| 50.00 | 3.13 | citric (2.48); lactic (1.31); propionic (0.03); |
| 50.00 | 1.56 | lactic (2.16); pyruvic (1.04); |
| 50.00 | 1.25 | benzoic (0.64); |
| 50.00 | 0.31 | benzoic (1.05); |
| 25.00 | 100.00 | butyric (0.04); propionic (0.09); pyruvic (0.65); |
| 25.00 | 50.00 | butyric (0.09); pyruvic (1.01); |
| 25.00 | 25.00 | pyruvic (0.78); |
| 25.00 | 12.50 | butyric (0.89); citric (1.98); |
| 25.00 | 6.25 | propionic (15.47); pyruvic (1.63); |
| 25.00 | 1.56 | lactic (2.01); pyruvic (6.28); |
| 25.00 | 1.25 | benzoic (6.29); |
| 25.00 | 0.63 | benzoic (1.77); |
| 25.00 | 0.31 | benzoic (1.37); |
| 12.50 | 100.00 | pyruvic (0.04); |
| 12.50 | 50.00 | propionic (0.18); pyruvic (1.47); |
| 12.50 | 25.00 | citric (0.14); |
| 12.50 | 12.50 | citric (5.38); propionic (1.55); |
| 12.50 | 6.25 | citric (4.56); propionic (10.9); |
| 12.50 | 3.13 | citric (10.86); octanoic (0.1); |
| 12.50 | 1.56 | lactic (3.34); octanoic (2.6); pyruvic (3.47); |
| 12.50 | 1.25 | benzoic (6.86); |
| 12.50 | 0.63 | benzoic (3.98); |
| 12.50 | 0.31 | benzoic (4.69); |
| 6.25 | 100.00 | butyric (0.39); pyruvic (0.36); |
| 6.25 | 50.00 | butyric (0.03); |
| 6.25 | 25.00 | butyric (0.06); |
| 6.25 | 12.50 | citric (4.01); |
| 6.25 | 6.25 | octanoic (2.59); propionic (4.13); |
| 6.25 | 3.13 | citric (2.27); octanoic (4.65); pyruvic (3.76); |
| 6.25 | 1.56 | lactic (2.01); octanoic (3.75); valeric (2.97); |
| 6.25 | 1.25 | benzoic (6.47); |
| 6.25 | 0.63 | benzoic (2.1); |
| 6.25 | 0.31 | sorbic (3.85); |
| 3.13 | 50.00 | butyric (0.06); pyruvic (0.15); |
| 3.13 | 25.00 | butyric (0.45); pyruvic (0.46); |
| 3.13 | 12.50 | citric (4.23); |
| 3.13 | 6.25 | propionic (8.62); |
| 3.13 | 3.13 | citric (1.62); |
| 3.13 | 1.56 | lactic (0.75); |
| 3.13 | 1.25 | benzoic (3.5); |
| 3.13 | 0.63 | sorbic (2.72); |
| 3.13 | 0.31 | sorbic (7.91); |
| 3.13 | 0.16 | sorbic (6.36); |
| 1.56 | 100.00 | pyruvic (0.09); valeric (0.3); |
| 1.56 | 25.00 | butyric (0.71); pyruvic (0.29); |
| 1.56 | 12.50 | citric (3.78); |
| 1.56 | 6.25 | propionic (10.39); |
| 1.56 | 3.13 | citric (7.5); lactic (1.93); octanoic (0.95); |
| 1.56 | 1.56 | octanoic (1.45); |
| 1.56 | 1.25 | benzoic (3.11); |
| 1.56 | 0.63 | benzoic (1.26); |
| 0.78 | 100.00 | butyric (0.2); formic (0.07); pyruvic (0.01); |
| 0.78 | 50.00 | pyruvic (0.27); |
| 0.78 | 25.00 | butyric (0.06); pyruvic (0.25); |
| 0.78 | 12.50 | citric (4.1); |
| 0.78 | 6.25 | lactic (6.27); octanoic (2.52); propionic (2.51); |
| 0.78 | 3.13 | citric (3.33); lactic (8.29); octanoic (3.81); |
| | | pyruvic (0.15); |
| 0.78 | 1.56 | citric (2.05); lactic (11.92); octanoic (5.55); |
| | | valeric (4.79); |
| 0.78 | 1.25 | benzoic (6.82); |
| 0.78 | 0.63 | benzoic (3.23); |
| 0.78 | 0.31 | benzoic (1.59); |
| 0.39 | 100.00 | butyric (0.38); formic (0.31); octanoic (1.67); |
| | | pyruvic (0.48); |
| 0.39 | 50.00 | formic (0.37); pyruvic (0.3); |
| 0.39 | 25.00 | butyric (0.1); |
| 0.39 | 12.50 | citric (1.22); octanoic (6.33); propionic (0.05); |
| 0.39 | 6.25 | citric (0.51); lactic (4.7); octanoic (10.88); |
| | | valeric (1.03); |
| 0.39 | 5.00 | benzoic (2.26); |
| 0.39 | 3.13 | citric (5.49); lactic (20.35); octanoic (9.99); |
| 0.39 | 1.56 | lactic (3.63); octanoic (8.91); |
| 0.39 | 1.25 | benzoic (2.15); |
| 0.20 | 100.00 | butyric (0.75); pyruvic (1.22); |
| 0.20 | 50.00 | formic (0); pyruvic (0.73); |
| 0.20 | 25.00 | butyric (0.23); pyruvic (0.54); |
| 0.20 | 12.50 | octanoic (3.12); propionic (0.61); |
| 0.20 | 6.25 | lactic (6.52); octanoic (4.22); |
| 0.20 | 3.13 | citric (4.8); lactic (1.26); octanoic (9.98); |
| 0.20 | 2.50 | benzoic (18.5); |
| 0.20 | 1.56 | lactic (0.08); octanoic (6.77); |
| 0.20 | 1.25 | benzoic (24.49); |
| 0.20 | 0.63 | benzoic (10.39); |
| 0.20 | 0.31 | benzoic (11.98); sorbic (0.29); |

TABLE 10
Synergistic inhibition of microbial growth of Shigella sonnei ATCC 29930, I virulent, WDCM 00127, CECT 4887 (DSMZ
5570) upon treatment with different concentrations of PHP in combination with different concentrations of organic acid(s) or salt(s)
thereof. In the details column, the tested organic acid(s) or salt(s) thereof is indicated followed by the observed “S” value in brackets.
Individual experimental results are separated by semicolon(s).

| PHP | acid/salt | |
|---|---|---|
| mM | mM | details |
| 100.00 | 100.00 | acetic (1.37); octanoic (16.32); propionic (0.09); succinic (0.51); valeric (0.65); |
| 100.00 | 50.00 | acetic (0.37); formic (0.11); octanoic (21.3); succinic (0.62); |
| 100.00 | 25.00 | acetic (0.57); butyric (0.32); octanoic (13.6); succinic (0.55); valeric (0.31); |
| 100.00 | 12.50 | acetic (14.08); butyric (0.5); formic (7.12); fumaric (17.84); octanoic (3.19); propionic (6.38); succinic |
| | | (24.43); |
| 100.00 | 10.00 | sorbic (7.51); |
| 100.00 | 6.25 | formic (7.03); fumaric (29.1); octanoic (7.69); propionic (4.26); succinic (67.99); |
| 100.00 | 5.00 | sorbic (8.39); |
| 100.00 | 3.13 | acetic (19.88); fumaric (22.28); octanoic (3.81); succinic (8.06); |
| 100.00 | 2.50 | sorbic (12.57); |
| 100.00 | 1.56 | fumaric (21.68); octanoic (13.75); |
| 100.00 | 1.25 | sorbic (21.83); |
| 100.00 | 0.78 | fumaric (28.64); |
| 100.00 | 0.63 | sorbic (16.11); |
| 100.00 | 0.39 | fumaric (27.39); |
| 100.00 | 0.31 | sorbic (14.33); |
| 100.00 | 0.16 | sorbic (12.73); |
| 50.00 | 100.00 | acetic (0.25); formic (0.67); octanoic (23.64); succinic (0.63); |
| 50.00 | 50.00 | octanoic (39.01); succinic (0.73); |
| 50.00 | 25.00 | butyric (0.54); octanoic (27.08); |
| 50.00 | 12.50 | acetic (12.32); butyric (1.99); formic (12.44); fumaric (21.12); octanoic (9.7); propionic (4.75); succinic |
| | | (26.53); valeric (0.01); |
| 50.00 | 6.25 | formic (20.87); fumaric (33.45); octanoic (14.61); succinic (83.95); |
| 50.00 | 5.00 | sorbic (8.12); |
| 50.00 | 3.13 | formic (20.89); fumaric (27.83); octanoic (13.02); succinic (30.05); |
| 50.00 | 2.50 | sorbic (8.24); |
| 50.00 | 1.56 | formic (24.55); fumaric (25.21); octanoic (29.92); succinic (24.2); |
| 50.00 | 0.78 | fumaric (31.61); |
| 50.00 | 0.63 | sorbic (18.25); |
| 50.00 | 0.31 | sorbic (22.33); |
| 50.00 | 0.16 | sorbic (32.06); |
| 25.00 | 100.00 | octanoic (26.47); succinic (0.54); valeric (0.32); |
| 25.00 | 50.00 | octanoic (43.77); valeric (0.37); |
| 25.00 | 25.00 | octanoic (30.87); succinic (0.33); valeric (0.05); |
| 25.00 | 12.50 | acetic (6.31); butyric (0.19); formic (10.57); fumaric (21.63); octanoic (15.85); propionic (5.25); |
| | | succinic (31.81); |
| 25.00 | 10.00 | sorbic (1.59); |
| 25.00 | 6.25 | formic (7.18); fumaric (28.27); octanoic (21.24); succinic (85.59); |
| 25.00 | 5.00 | sorbic (11.97); |
| 25.00 | 3.13 | fumaric (26.58); octanoic (28.3); succinic (66.87); |
| 25.00 | 2.50 | sorbic (21.47); |
| 25.00 | 1.56 | acetic (3.81); formic (25.19); fumaric (22.36); octanoic (35.62); succinic (37.81); |
| 25.00 | 0.78 | fumaric (32.06); |
| 25.00 | 0.39 | fumaric (30.78); |
| 25.00 | 0.16 | sorbic (47.35); |
| 12.50 | 100.00 | formic (0.1); octanoic (25.54); succinic (0.68); |
| 12.50 | 50.00 | octanoic (44.85); succinic (0.32); valeric (0.16); |
| 12.50 | 25.00 | octanoic (27.79); |
| 12.50 | 12.50 | acetic (7.07); formic (8.65); fumaric (22); octanoic (13.95); propionic (5.06); succinic (30.45); |
| 12.50 | 6.25 | formic (42.89); fumaric (33.22); octanoic (19.33); succinic (105.47); |
| 12.50 | 5.00 | sorbic (20.04); |
| 12.50 | 3.13 | acetic (3.2); formic (6.83); fumaric (31.1); octanoic (25.55); succinic (38.2); |
| 12.50 | 2.50 | sorbic (14.91); |
| 12.50 | 1.56 | acetic (12.77); fumaric (28.23); octanoic (37.76); succinic (41.98); |
| 12.50 | 0.78 | fumaric (37.13); |
| 12.50 | 0.63 | sorbic (28.69); |
| 12.50 | 0.39 | fumaric (40.24); |
| 12.50 | 0.31 | sorbic (28.68); |
| 12.50 | 0.16 | sorbic (23.66); |
| 6.25 | 100.00 | octanoic (29.68); succinic (1.34); valeric (0.17); |
| 6.25 | 50.00 | octanoic (46.11); succinic (0.55); |
| 6.25 | 25.00 | octanoic (35.28); succinic (0.61); |
| 6.25 | 12.50 | acetic (5.12); formic (6.78); fumaric (24.69); octanoic (16.29); propionic (4.52); succinic (36.83); valeric |
| | | (0.92); |
| 6.25 | 10.00 | sorbic (6.34); |
| 6.25 | 6.25 | acetic (2.13); formic (40.7); fumaric (34.03); octanoic (24.51); propionic (3.02); succinic (89.84); |
| 6.25 | 5.00 | sorbic (32.85); |
| 6.25 | 3.13 | acetic (13.66); butyric (3.24); fumaric (30.5); octanoic (36.02); succinic (88.87); |
| 6.25 | 2.50 | sorbic (24.21); |
| 6.25 | 1.56 | acetic (28.99); butyric (6.02); fumaric (23.84); octanoic (40.4); succinic (93.75); |
| 6.25 | 1.25 | sorbic (21.74); |
| 6.25 | 0.78 | fumaric (47.31); |
| 6.25 | 0.63 | sorbic (51.86); |
| 6.25 | 0.39 | fumaric (50.67); |
| 6.25 | 0.31 | sorbic (19.09); |
| 6.25 | 0.16 | sorbic (86.92); |
| 3.13 | 100.00 | octanoic (20.04); succinic (0.71); valeric (0.56); |
| 3.13 | 50.00 | octanoic (33.34); succinic (0.82); |
| 3.13 | 25.00 | octanoic (19.33); |
| 3.13 | 12.50 | acetic (4.13); formic (6.55); fumaric (25.61); octanoic (4.25); propionic (5.81); succinic (33.76); valeric |
| | | (0.26); |
| 3.13 | 10.00 | sorbic (4.57); |
| 3.13 | 6.25 | acetic (2.5); formic (21.1); fumaric (30.65); octanoic (7.9); succinic (112.96); |
| 3.13 | 5.00 | sorbic (35.36); |
| 3.13 | 3.13 | acetic (28.91); butyric (0.45); fumaric (23.98); octanoic (31.04); propionic (2.88); succinic (113.97); |
| 3.13 | 2.50 | sorbic (42.34); |
| 3.13 | 1.56 | acetic (25.51); butyric (2.68); fumaric (36.15); octanoic (30.54); propionic (1.37); succinic (99.92); |
| 3.13 | 1.25 | sorbic (36.58); |
| 3.13 | 0.78 | fumaric (50.43); |
| 3.13 | 0.63 | sorbic (34.59); |
| 3.13 | 0.39 | fumaric (55.42); |
| 3.13 | 0.31 | sorbic (26.7); |
| 3.13 | 0.16 | sorbic (92.17); |
| 1.56 | 100.00 | octanoic (26.5); |
| 1.56 | 50.00 | octanoic (46.08); |
| 1.56 | 25.00 | octanoic (26.92); succinic (0.77); |
| 1.56 | 12.50 | acetic (5.04); formic (6.54); fumaric (26.14); octanoic (3.35); propionic (6.68); succinic (31.53); |
| 1.56 | 6.25 | acetic (3.34); formic (9.3); fumaric (32.58); octanoic (22.42); propionic (2.14); succinic (103.3); |
| 1.56 | 5.00 | sorbic (14.96); |
| 1.56 | 3.13 | acetic (21.63); butyric (4.02); fumaric (26.49); octanoic (31.95); propionic (2.91); succinic (9.58); |
| 1.56 | 2.50 | sorbic (19.44); |
| 1.56 | 1.56 | acetic (26.45); butyric (2.32); fumaric (39.5); propionic (7.82); succinic (98.08); |
| 1.56 | 0.78 | fumaric (48.14); |
| 1.56 | 0.63 | sorbic (3.58); |
| 1.56 | 0.39 | fumaric (49.89); |
| 1.56 | 0.16 | sorbic (65.52); |
| 0.78 | 100.00 | octanoic (34.45); succinic (0.93); valeric (0.06); |
| 0.78 | 50.00 | octanoic (48.89); succinic (0.56); |
| 0.78 | 25.00 | octanoic (33.31); succinic (1.23); |
| 0.78 | 12.50 | acetic (5.03); formic (5.55); fumaric (26.3); octanoic (12.79); propionic (6.41); succinic (33.1); |
| 0.78 | 6.25 | formic (18.83); fumaric (34.23); octanoic (30.03); propionic (1.8); succinic (113.35); |
| 0.78 | 5.00 | sorbic (18.69); |
| 0.78 | 3.13 | acetic (23.59); butyric (6.35); fumaric (31.61); octanoic (41.87); propionic (3.96); succinic (114.86); |
| 0.78 | 2.50 | sorbic (23.26); |
| 0.78 | 1.56 | acetic (25.56); butyric (5.89); fumaric (40.2); propionic (6.59); succinic (101); |
| 0.78 | 0.78 | fumaric (48.66); |
| 0.78 | 0.39 | fumaric (50.68); |
| 0.78 | 0.31 | sorbic (42.24); |
| 0.78 | 0.16 | sorbic (53.08); |
| 0.39 | 100.00 | formic (0.01); octanoic (27.09); valeric (0.2); |
| 0.39 | 50.00 | octanoic (44.72); succinic (0.31); |
| 0.39 | 25.00 | octanoic (30.42); succinic (0.45); |
| 0.39 | 12.50 | acetic (7.27); formic (4.32); fumaric (23.41); octanoic (10.33); propionic (11.14); succinic (32.13); |
| | | valeric (0.57); |
| 0.39 | 6.25 | formic (26.23); fumaric (38.94); octanoic (11.13); propionic (2.79); succinic (36.73); |
| 0.39 | 5.00 | sorbic (22.04); |
| 0.39 | 3.13 | acetic (27.58); butyric (5.93); fumaric (34.29); octanoic (41.91); propionic (8.26); succinic (18.85); |
| 0.39 | 2.50 | sorbic (34.8); |
| 0.39 | 1.56 | acetic (23.13); butyric (1.86); fumaric (42.25); propionic (11.58); succinic (77.71); |
| 0.39 | 0.78 | fumaric (52.88); |
| 0.39 | 0.63 | sorbic (13.74); |
| 0.39 | 0.39 | fumaric (55.06); |
| 0.39 | 0.31 | sorbic (1.46); |
| 0.39 | 0.16 | sorbic (56); |
| 0.20 | 100.00 | octanoic (37.3); succinic (1.43); valeric (0.39); |
| 0.20 | 50.00 | octanoic (66.52); succinic (0.46); |
| 0.20 | 25.00 | octanoic (49.47); succinic (0.06); |
| 0.20 | 12.50 | acetic (9.36); fumaric (35.96); octanoic (19.6); propionic (11.4); succinic (32); valeric (3.28); |
| 0.20 | 6.25 | acetic (7.22); butyric (9.37); formic (36.1); fumaric (48.52); octanoic (26.98); propionic (4.89); succinic |
| | | (73.04); valeric (3.41); |
| 0.20 | 5.00 | sorbic (11.72); |
| 0.20 | 3.13 | acetic (27.47); butyric (15.95); fumaric (54.76); octanoic (69.52); propionic (1.27); succinic (67.04); |
| 0.20 | 2.50 | sorbic (38.81); |
| 0.20 | 1.56 | butyric (10.22); fumaric (40.55); butyric (49.26); |
| 0.20 | 0.78 | fumaric (69.17); |
| 0.20 | 0.63 | sorbic (3.69); |
| 0.20 | 0.39 | fumaric (86.39); |
| 0.20 | 0.16 | sorbic (39.15); |

TABLE 11
Synergistic inhibition of microbial growth of Clostridium perfringens (CCUG 47895; type A as described in Johansson et al.
2004. Vet Microbiol. 99(3-4): 251-257) upon treatment with different concentrations of SHP in combination with different
concentrations of organic acid(s) or salt(s) thereof. In the details column, the tested organic acid(s) or salt(s) thereof is indicated
followed by the observed “S” value in brackets. Individual experimental results are separated by semicolon(s).

| SHP | acid/salt | |
|---|---|---|
| mM | mM | details |
| 100.00 | 100.00 | formic + acetic + propionic (0.4); |
| 100.00 | 50.00 | formic + acetic + propionic (0.2); formic (6.84); |
| 100.00 | 25.00 | formic + acetic + propionic (0.57); sodium benzoate (6.07); propionic (5.02); |
| 100.00 | 20.00 | formic + acetic + propionic + benzoic (4.19); |
| 100.00 | 12.50 | formic + acetic + propionic (0.15); formic (10.04); sodium benzoate (13.17); |
| 100.00 | 10.00 | formic + acetic + propionic + benzoic (24.89); |
| 100.00 | 6.25 | formic + acetic + propionic (3.76); sodium benzoate (3.6); |
| 100.00 | 3.13 | propionic (33.28); |
| 100.00 | 1.56 | formic (0.26); propionic (38.56); |
| 100.00 | 1.25 | benzoic (0); |
| 50.00 | 400.00 | sodium benzoate (0.1); |
| 50.00 | 200.00 | sodium benzoate (0.2); |
| 50.00 | 100.00 | formic + acetic + propionic + benzoic (0.02); |
| 50.00 | 50.00 | formic (10.46); sodium benzoate (2.49); |
| 50.00 | 40.00 | formic + acetic + propionic + benzoic (0.17); |
| 50.00 | 25.00 | calcium formate (3.26); sodium benzoate (16.56); propionic (0.28); |
| 50.00 | 20.00 | formic + acetic + propionic + benzoic (4.48); |
| 50.00 | 12.50 | formic + acetic + propionic (2.63); sodium benzoate (18.96); |
| 50.00 | 10.00 | formic + acetic + propionic + benzoic (33.58); |
| 50.00 | 6.25 | calcium formate (3.43); sodium benzoate (34.39); |
| 50.00 | 5.00 | formic + acetic + propionic + benzoic (8.68); |
| 50.00 | 3.13 | propionic (7.52); |
| 50.00 | 2.50 | formic + acetic + propionic + benzoic (34.9); |
| 50.00 | 1.56 | acetic (22.41); propionic (1.79); |
| 50.00 | 0.63 | benzoic (7.21); |
| 25.00 | 400.00 | sodium benzoate (0.07); |
| 25.00 | 200.00 | sodium benzoate (0.44); |
| 25.00 | 100.00 | formic (2.97); sodium benzoate (0.24); propionic (2.5); |
| 25.00 | 80.00 | formic + acetic + propionic + benzoic (0.1); |
| 25.00 | 50.00 | formic (12.2); sodium benzoate (2.09); propionic (1.62); |
| 25.00 | 40.00 | formic + acetic + propionic + benzoic (0.57); |
| 25.00 | 25.00 | calcium formate (2.36); sodium benzoate (17.78); propionic (21.83); |
| 25.00 | 20.00 | formic + acetic + propionic + benzoic (10.4); |
| 25.00 | 12.50 | formic (36.12); sodium benzoate (28.38); propionic (4.91); |
| 25.00 | 10.00 | formic + acetic + propionic + benzoic (44.66); |
| 25.00 | 6.25 | formic (3.4); sodium benzoate (11.66); |
| 25.00 | 3.13 | formic (44.01); propionic (58.57); |
| 25.00 | 2.50 | formic + acetic + propionic + benzoic (29.36); |
| 25.00 | 1.56 | formic (46.87); propionic (37.93); |
| 12.50 | 400.00 | sodium benzoate (0.1); |
| 12.50 | 200.00 | calcium formate (3.49); sodium benzoate (0.45); |
| 12.50 | 100.00 | formic (3.35); sodium benzoate (0.41); propionic (0.05); |
| 12.50 | 80.00 | formic + acetic + propionic + benzoic (0.23); |
| 12.50 | 50.00 | formic (22.58); sodium benzoate (2.85); |
| 12.50 | 40.00 | formic + acetic + propionic + benzoic (0.79); |
| 12.50 | 25.00 | formic + acetic + propionic (0.21); sodium benzoate (17.82); propionic (13.33); |
| 12.50 | 20.00 | benzoic (1.66); formic + acetic + propionic + benzoic (3.08); |
| 12.50 | 12.50 | formic (10.94); sodium benzoate (10.83); |
| 12.50 | 10.00 | formic + acetic + propionic + benzoic (49.6); |
| 12.50 | 6.25 | formic (0.68); sodium benzoate (29.58); propionic (7.21); |
| 12.50 | 5.00 | formic + acetic + propionic + benzoic (10.13); |
| 12.50 | 3.13 | propionic (7.83); |
| 12.50 | 2.50 | formic + acetic + propionic + benzoic (46.89); |
| 12.50 | 1.56 | formic (11.83); |
| 12.50 | 1.25 | formic + acetic + propionic + benzoic (22.94); |
| 6.25 | 400.00 | sodium benzoate (0.36); |
| 6.25 | 200.00 | calcium formate (18.56); sodium benzoate (0.49); |
| 6.25 | 100.00 | formic (2.34); sodium benzoate (0.47); propionic (6.11); |
| 6.25 | 80.00 | formic + acetic + propionic + benzoic (0.41); |
| 6.25 | 50.00 | formic (12.41); sodium benzoate (2.44); propionic (3.59); |
| 6.25 | 40.00 | formic + acetic + propionic + benzoic (0.47); |
| 6.25 | 25.00 | formic + acetic + propionic (0.11); calcium formate (7.53); sodium benzoate (6.87); propionic (41.61); |
| 6.25 | 20.00 | benzoic (6.22); formic + acetic + propionic + benzoic (7.49); |
| 6.25 | 12.50 | acetic (5.54); formic (43.92); sodium benzoate (4.85); propionic (31.22); |
| 6.25 | 10.00 | formic + acetic + propionic + benzoic (28.19); |
| 6.25 | 6.25 | formic + acetic + propionic (4.96); calcium formate (9.52); formic (3.66); sodium benzoate (19.68); |
| | | propionic (12); |
| 6.25 | 5.00 | benzoic (9.14); |
| 6.25 | 3.13 | formic (24.7); propionic (48.14); |
| 6.25 | 2.50 | formic + acetic + propionic + benzoic (23.85); |
| 6.25 | 1.56 | propionic (11.59); |
| 3.13 | 200.00 | calcium formate (11.88); |
| 3.13 | 100.00 | calcium formate (2.44); formic (1.61); propionic (2.83); |
| 3.13 | 50.00 | formic (16.83); propionic (1.35); |
| 3.13 | 25.00 | calcium formate (3.75); propionic (32.93); |
| 3.13 | 20.00 | benzoic (5.44); |
| 3.13 | 12.50 | formic (1.98); propionic (7.85); |
| 3.13 | 10.00 | formic + acetic + propionic + benzoic (11.28); |
| 3.13 | 6.25 | formic (29.22); propionic (24.49); |
| 3.13 | 5.00 | benzoic (28.07); |
| 3.13 | 3.13 | formic (33.97); propionic (30.77); |
| 3.13 | 2.50 | formic + acetic + propionic + benzoic (26.76); |
| 3.13 | 1.56 | formic (3.15); propionic (24.52); |
| 3.13 | 1.25 | benzoic (6.79); |
| 1.56 | 200.00 | calcium formate (11.73); |
| 1.56 | 100.00 | acetic (0.04); formic (3.84); propionic (5.11); |
| 1.56 | 80.00 | formic + acetic + propionic + benzoic (0.42); |
| 1.56 | 50.00 | formic (19.35); propionic (4.03); |
| 1.56 | 40.00 | formic + acetic + propionic + benzoic (0.34); |
| 1.56 | 25.00 | calcium formate (13.6); propionic (33.5); |
| 1.56 | 20.00 | benzoic (4.85); formic + acetic + propionic + benzoic (10.35); |
| 1.56 | 12.50 | acetic (3.87); formic (28.96); |
| 1.56 | 10.00 | formic + acetic + propionic + benzoic (30.73); |
| 1.56 | 6.25 | calcium formate (8.24); formic (0.04); propionic (37.52); |
| 1.56 | 5.00 | benzoic (1.14); formic + acetic + propionic + benzoic (0.53); |
| 1.56 | 3.13 | acetic (12.37); formic (35.96); propionic (71.87); |
| 1.56 | 2.50 | formic + acetic + propionic + benzoic (29.83); |
| 1.56 | 1.56 | propionic (56.35); |
| 0.78 | 200.00 | calcium formate (9.24); |
| 0.78 | 100.00 | calcium formate (1.97); formic (2.9); |
| 0.78 | 50.00 | formic (18.35); |
| 0.78 | 25.00 | calcium formate (3.71); |
| 0.78 | 20.00 | formic + acetic + propionic + benzoic (8.39); |
| 0.78 | 12.50 | formic (18.13); |
| 0.78 | 10.00 | formic + acetic + propionic + benzoic (32.22); |
| 0.78 | 6.25 | calcium formate (6.73); |
| 0.78 | 5.00 | benzoic (6.38); |
| 0.78 | 1.56 | formic (2.63); |
| 0.39 | 200.00 | calcium formate (3.52); |
| 0.39 | 100.00 | formic + acetic + propionic (0.18); formic (2.32); propionic (0.86); |
| 0.39 | 80.00 | formic + acetic + propionic + benzoic (0.09); |
| 0.39 | 50.00 | formic + acetic + propionic (0.12); calcium formate (2.23); formic (19.62); propionic (0.53); |
| 0.39 | 40.00 | formic + acetic + propionic + benzoic (0.3); |
| 0.39 | 25.00 | formic + acetic + propionic (0.52); propionic (5.36); |
| 0.39 | 20.00 | formic + acetic + propionic + benzoic (9.95); |
| 0.39 | 12.50 | acetic (1.32); formic (1.42); |
| 0.39 | 10.00 | formic + acetic + propionic + benzoic (21.87); |
| 0.39 | 6.25 | calcium formate (0.47); |
| 0.39 | 5.00 | benzoic (41.37); formic + acetic + propionic + benzoic (1.36); |
| 0.39 | 3.13 | acetic (26.67); propionic (17.45); |
| 0.39 | 2.50 | formic + acetic + propionic + benzoic (32.09); |
| 0.39 | 1.56 | formic (8.53); |
| 0.39 | 0.63 | benzoic (0.5); |
| 0.20 | 200.00 | calcium formate (4.06); |
| 0.20 | 100.00 | acetic (1.65); formic + acetic + propionic (0.48); calcium formate (5.09); propionic (0.36); |
| 0.20 | 50.00 | acetic (0.23); formic + acetic + propionic (0.71); calcium formate (3.09); formic (12.71); sodium benzoate |
| | | (0.23); propionic (0.56); |
| 0.20 | 25.00 | formic + acetic + propionic (0.67); calcium formate (12.7); |
| 0.20 | 20.00 | formic + acetic + propionic + benzoic (7.25); |
| 0.20 | 12.50 | acetic (7.26); formic + acetic + propionic (4.31); |
| 0.20 | 10.00 | formic + acetic + propionic + benzoic (6.5); |
| 0.20 | 6.25 | formic (3.55); |
| 0.20 | 5.00 | formic + acetic + propionic + benzoic (1.48); |
| 0.20 | 3.13 | acetic (7.24); |
| 0.20 | 2.50 | formic + acetic + propionic + benzoic (21.04); |

TABLE 12
Synergistic inhibition of microbial growth of Clostridium perfringens (CCUG 47895; type A as described in Johansson et al.
2004. Vet Microbiol. 99(3-4): 251-257) upon treatment with different concentrations of MHP in combination with different
concentrations of organic acid(s) or salt(s) thereof. In the details column, the tested organic acid(s) or salt(s) thereof is indicated
followed by the observed “S” value in brackets. Individual experimental results are separated by semicolon(s).

| MHP | acid/salt | |
|---|---|---|
| mM | mM | details |
| 100.00 | 400.00 | sodium benzoate (0.82); |
| 100.00 | 200.00 | sodium benzoate (1.1); |
| 100.00 | 100.00 | formic + acetic + propionic (1.78); calcium formate (15.12); sodium benzoate (1.41); |
| 100.00 | 80.00 | formic + acetic + propionic + benzoic (3.78); |
| 100.00 | 50.00 | formic + acetic + propionic (2.01); calcium formate (3.05); sodium benzoate (3.1); |
| 100.00 | 40.00 | formic + acetic + propionic + benzoic (3.51); |
| 100.00 | 25.00 | formic + acetic + propionic (2.03); sodium benzoate (25.13); |
| 100.00 | 20.00 | formic + acetic + propionic + benzoic (16.84); |
| 100.00 | 12.50 | formic + acetic + propionic (9.51); calcium formate (1.62); sodium benzoate (47.07); |
| 100.00 | 10.00 | formic + acetic + propionic + benzoic (15.83); |
| 100.00 | 6.25 | sodium benzoate (44.55); |
| 100.00 | 5.00 | benzoic (1.82); formic + acetic + propionic + benzoic (50.52); |
| 100.00 | 2.50 | formic + acetic + propionic + benzoic (26.75); |
| 100.00 | 1.56 | propionic (9.65); |
| 100.00 | 1.25 | benzoic (37.42); formic + acetic + propionic + benzoic (33.01); |
| 100.00 | 0.63 | benzoic (23.55); |
| 100.00 | 0.31 | benzoic (21.48); |
| 50.00 | 400.00 | sodium benzoate (0.19); |
| 50.00 | 200.00 | sodium benzoate (0.37); |
| 50.00 | 100.00 | formic + acetic + propionic (1.22); calcium formate (12.49); formic (2.84); sodium benzoate (0.68); |
| 50.00 | 80.00 | formic + acetic + propionic + benzoic (1.65); |
| 50.00 | 50.00 | formic + acetic + propionic (1.65); calcium formate (23.52); sodium benzoate (2.59); |
| 50.00 | 40.00 | formic + acetic + propionic + benzoic (1.65); |
| 50.00 | 25.00 | formic + acetic + propionic (1.72); calcium formate (6.53); formic (1.81); sodium benzoate (21.49); |
| 50.00 | 20.00 | formic + acetic + propionic + benzoic (9.16); |
| 50.00 | 12.50 | formic + acetic + propionic (2.51); calcium formate (10.82); sodium benzoate (40.43); |
| 50.00 | 10.00 | formic + acetic + propionic + benzoic (15); |
| 50.00 | 6.25 | sodium benzoate (33.67); |
| 50.00 | 5.00 | benzoic (2.28); formic + acetic + propionic + benzoic (24.6); |
| 50.00 | 1.56 | propionic (13.59); |
| 50.00 | 1.25 | benzoic (42.17); formic + acetic + propionic + benzoic (1.54); |
| 50.00 | 0.63 | benzoic (9.23); |
| 50.00 | 0.31 | benzoic (4.22); |
| 25.00 | 400.00 | sodium benzoate (0.15); |
| 25.00 | 200.00 | sodium benzoate (0.77); |
| 25.00 | 100.00 | calcium formate (3.54); sodium benzoate (0.98); propionic (8.53); |
| 25.00 | 80.00 | formic + acetic + propionic + benzoic (0.35); |
| 25.00 | 50.00 | formic + acetic + propionic (0.71); calcium formate (3.48); sodium benzoate (2.79); propionic (23.64); |
| 25.00 | 40.00 | formic + acetic + propionic + benzoic (0.91); |
| 25.00 | 25.00 | formic + acetic + propionic (0.75); sodium benzoate (27.17); propionic (13.4); |
| 25.00 | 20.00 | formic + acetic + propionic + benzoic (9.87); |
| 25.00 | 12.50 | calcium formate (7.43); sodium benzoate (48.37); propionic (34.98); |
| 25.00 | 10.00 | formic + acetic + propionic + benzoic (4.55); |
| 25.00 | 6.25 | calcium formate (17.15); sodium benzoate (32.59); propionic (38.72); |
| 25.00 | 5.00 | formic + acetic + propionic + benzoic (0.46); |
| 25.00 | 3.13 | propionic (32.77); |
| 25.00 | 2.50 | benzoic (7.58); |
| 25.00 | 1.56 | propionic (81.18); |
| 25.00 | 1.25 | benzoic (13.1); |
| 25.00 | 0.63 | benzoic (5.37); |
| 25.00 | 0.31 | benzoic (15.9); |
| 12.50 | 400.00 | sodium benzoate (0.11); |
| 12.50 | 200.00 | calcium formate (0.54); sodium benzoate (0.55); |
| 12.50 | 100.00 | calcium formate (1.02); sodium benzoate (0.8); |
| 12.50 | 80.00 | formic + acetic + propionic + benzoic (1.52); |
| 12.50 | 50.00 | acetic (3.23); formic + acetic + propionic (0.51); sodium benzoate (2.66); propionic (2.44); |
| 12.50 | 40.00 | formic + acetic + propionic + benzoic (1.64); |
| 12.50 | 25.00 | formic + acetic + propionic (0.29); sodium benzoate (25.16); |
| 12.50 | 20.00 | formic + acetic + propionic + benzoic (10.27); |
| 12.50 | 12.50 | formic + acetic + propionic (4.06); calcium formate (4.8); sodium benzoate (36.95); |
| 12.50 | 10.00 | formic + acetic + propionic + benzoic (27.52); |
| 12.50 | 6.25 | calcium formate (15.13); sodium benzoate (28.89); |
| 12.50 | 5.00 | formic + acetic + propionic + benzoic (39.83); |
| 12.50 | 2.50 | formic + acetic + propionic + benzoic (35.13); |
| 12.50 | 1.25 | benzoic (11.13); formic + acetic + propionic + benzoic (20.05); |
| 12.50 | 0.63 | benzoic (20.22); |
| 12.50 | 0.31 | benzoic (10.35); |
| 6.25 | 400.00 | sodium benzoate (0.51); |
| 6.25 | 200.00 | calcium formate (2.6); sodium benzoate (0.57); |
| 6.25 | 100.00 | acetic (4.01); formic + acetic + propionic (0.8); formic (1.4); sodium benzoate (0.87); propionic (24.27); |
| 6.25 | 80.00 | formic + acetic + propionic + benzoic (0.98); |
| 6.25 | 50.00 | acetic (8.74); formic + acetic + propionic (1.24); formic (0.31); sodium benzoate (1.08); propionic (41.78); |
| 6.25 | 40.00 | formic + acetic + propionic + benzoic (0.78); |
| 6.25 | 25.00 | acetic (23.77); formic + acetic + propionic (1.51); calcium formate (1.07); sodium benzoate (23.56); |
| | | propionic (65.38); |
| 6.25 | 20.00 | formic + acetic + propionic + benzoic (6.59); |
| 6.25 | 12.50 | acetic (17.4); formic + acetic + propionic (5.96); sodium benzoate (25.38); propionic (71.12); |
| 6.25 | 10.00 | formic + acetic + propionic + benzoic (21.06); |
| 6.25 | 6.25 | calcium formate (2.44); formic (1.61); sodium benzoate (25.06); propionic (24.02); |
| 6.25 | 5.00 | formic + acetic + propionic + benzoic (15.63); |
| 6.25 | 3.13 | formic + acetic + propionic (34.08); formic (19.77); propionic (4.64); |
| 6.25 | 2.50 | formic + acetic + propionic + benzoic (17.25); |
| 6.25 | 1.56 | propionic (84.83); |
| 6.25 | 1.25 | benzoic (7.69); formic + acetic + propionic + benzoic (0.4); |
| 6.25 | 0.63 | benzoic (7.07); |
| 6.25 | 0.31 | benzoic (10.48); |
| 3.13 | 100.00 | acetic (0.53); formic + acetic + propionic (0.48); calcium formate (4.52); |
| 3.13 | 80.00 | formic + acetic + propionic + benzoic (0.77); |
| 3.13 | 50.00 | acetic (3.54); formic + acetic + propionic (0.85 propionic (7.35); |
| 3.13 | 40.00 | ); formic + acetic + propionic + benzoic (0.69); |
| 3.13 | 25.00 | formic + acetic + propionic (0.72); sodium benzoate (3.46); |
| 3.13 | 6.25 | formic + acetic + propionic (12.13); |
| 3.13 | 5.00 | benzoic (3.59); formic + acetic + propionic + benzoic (0.83); |
| 3.13 | 3.13 | acetic (0.69); |
| 3.13 | 2.50 | formic + acetic + propionic + benzoic (6.05); |
| 3.13 | 1.56 | formic + acetic + propionic (2.61); propionic (1.31); |
| 3.13 | 1.25 | benzoic (21.91); |
| 3.13 | 0.63 | benzoic (28.9); |
| 3.13 | 0.31 | benzoic (2.16); |
| 1.56 | 200.00 | calcium formate (1.57); |
| 1.56 | 100.00 | acetic (0.32); formic + acetic + propionic (0.27); calcium formate (2.37); |
| 1.56 | 80.00 | formic + acetic + propionic + benzoic (1.4); |
| 1.56 | 50.00 | acetic (4.33); formic + acetic + propionic (0.61); |
| 1.56 | 40.00 | formic + acetic + propionic + benzoic (1.09); |
| 1.56 | 25.00 | acetic (6); formic + acetic + propionic (0.67); |
| 1.56 | 20.00 | formic + acetic + propionic + benzoic (12.53); |
| 1.56 | 12.50 | formic + acetic + propionic + benzoic (8.8); |
| 1.56 | 6.25 | calcium formate (15.93); |
| 1.56 | 5.00 | formic + acetic + propionic + benzoic (47.91); |
| 1.56 | 2.50 | formic + acetic + propionic + benzoic (28.59); |
| 1.56 | 1.25 | formic + acetic + propionic + benzoic (5.75); |
| 0.78 | 200.00 | calcium formate (1.34); |
| 0.78 | 100.00 | acetic (0.07); calcium formate (9.53); propionic (19.18); |
| 0.78 | 80.00 | formic + acetic + propionic + benzoic (0.21); |
| 0.78 | 50.00 | acetic (3.61); formic + acetic + propionic (0.24); propionic (35.64); |
| 0.78 | 40.00 | formic + acetic + propionic + benzoic (0.28); |
| 0.78 | 25.00 | formic + acetic + propionic (0.14); calcium formate (0.18); sodium benzoate (5.2); propionic (38.97); |
| 0.78 | 20.00 | formic + acetic + propionic + benzoic (2.88); |
| 0.78 | 12.50 | propionic (76.58); |
| 0.78 | 10.00 | formic + acetic + propionic + benzoic (12.84); |
| 0.78 | 6.25 | calcium formate (5.08); propionic (11.83); |
| 0.78 | 5.00 | formic + acetic + propionic + benzoic (21.63); |
| 0.78 | 1.56 | propionic (53.67); |
| 0.78 | 1.25 | benzoic (21.2); |
| 0.78 | 0.63 | benzoic (15.51); |
| 0.78 | 0.31 | benzoic (3.53); |
| 0.39 | 100.00 | formic + acetic + propionic (0.28); calcium formate (0.92); formic (0.06); propionic (18.75); |
| 0.39 | 80.00 | formic + acetic + propionic + benzoic (0.64); |
| 0.39 | 50.00 | acetic (1.62); formic + acetic + propionic (0.83); propionic (36.68); |
| 0.39 | 40.00 | formic + acetic + propionic + benzoic (0.57); |
| 0.39 | 25.00 | formic + acetic + propionic (1.01); propionic (57.42); |
| 0.39 | 20.00 | formic + acetic + propionic + benzoic (9.65); |
| 0.39 | 12.50 | formic + acetic + propionic (7);propionic (76.38); |
| 0.39 | 10.00 | formic + acetic + propionic + benzoic (12.74); |
| 0.39 | 6.25 | calcium formate (0.18); |
| 0.39 | 5.00 | formic + acetic + propionic + benzoic (47.73); |
| 0.39 | 3.13 | formic + acetic + propionic (3.52); propionic (26.72); |
| 0.39 | 2.50 | formic + acetic + propionic + benzoic (20.34); |
| 0.39 | 1.56 | formic + acetic + propionic (0.77); propionic (68.09); |
| 0.39 | 0.63 | benzoic (15.9); |
| 0.20 | 100.00 | acetic (1.73); calcium formate (14.7); propionic (0.84); |
| 0.20 | 80.00 | formic + acetic + propionic + benzoic (0.49); |
| 0.20 | 50.00 | acetic (4.66); formic + acetic + propionic (0.63); calcium formate (9.64); propionic (16.34); |
| 0.20 | 40.00 | formic + acetic + propionic + benzoic (0.72); |
| 0.20 | 25.00 | formic + acetic + propionic (0.35); sodium benzoate (8.11); |
| 0.20 | 20.00 | formic + acetic + propionic + benzoic (4.9); |
| 0.20 | 12.50 | acetic (11.43); propionic (20.35); |
| 0.20 | 10.00 | formic + acetic + propionic + benzoic (18.15); |
| 0.20 | 6.25 | formic + acetic + propionic (5.41); calcium formate (1.77); |
| 0.20 | 5.00 | formic + acetic + propionic + benzoic (27.7); |
| 0.20 | 3.13 | formic + acetic + propionic (2.68); |
| 0.20 | 2.50 | formic + acetic + propionic + benzoic (6.55); |
| 0.20 | 1.56 | formic + acetic + propionic (2.75); propionic (6.85); |
| 0.20 | 1.25 | benzoic (6.94); |
| 0.20 | 0.63 | benzoic (24.72); |

TABLE 13
Synergistic inhibition of microbial growth of Clostridium perfringens (CCUG 47895; type A as described in Johansson et al.
2004. Vet Microbiol. 99(3-4): 251-257) upon treatment with different concentrations of PHP in combination with different
concentrations of organic acid(s) or salt(s) thereof. In the details column, the tested organic acid(s) or salt(s) thereof is
indicated followed by the observed “S” value in brackets. Individual experimental results are separated by
semicolon(s).

| PHP | acid/salt | |
|---|---|---|
| mM | mM | details |
| 100.00 | 100.00 | formic + acetic + propionic (0.8); |
| 100.00 | 80.00 | formic + acetic + propionic + benzoic (0.49); |
| 100.00 | 50.00 | formic + acetic + propionic (0.71); calcium formate (16.03); sodium benzoate (0.01); |
| 100.00 | 40.00 | formic + acetic + propionic + benzoic (0.41); |
| 100.00 | 25.00 | acetic (1.31); formic + acetic + propionic (0.69); |
| 100.00 | 20.00 | formic + acetic + propionic + benzoic (9.99); |
| 100.00 | 12.50 | sodium benzoate (4.63); |
| 100.00 | 6.25 | formic + acetic + propionic (2.55); sodium benzoate (3.55); propionic (1.64); |
| 100.00 | 3.13 | acetic (16.07); |
| 100.00 | 1.56 | acetic (24.61); |
| 50.00 | 100.00 | formic + acetic + propionic (0.6); sodium benzoate (0.05); |
| 50.00 | 80.00 | formic + acetic + propionic + benzoic (0.17); |
| 50.00 | 50.00 | formic + acetic + propionic (0.86); calcium formate (8.16); |
| 50.00 | 40.00 | formic + acetic + propionic + benzoic (0.38); |
| 50.00 | 25.00 | formic + acetic + propionic (0.84); |
| 50.00 | 20.00 | formic + acetic + propionic + benzoic (11.18); |
| 50.00 | 6.25 | formic + acetic + propionic (29.6); sodium benzoate (9.7); |
| 50.00 | 5.00 | formic + acetic + propionic + benzoic (21.39); |
| 50.00 | 3.13 | acetic (4.42); |
| 50.00 | 2.50 | formic + acetic + propionic + benzoic (0.24); |
| 50.00 | 1.56 | acetic (12.45); formic (18.84); |
| 50.00 | 0.31 | benzoic (17.76); |
| 25.00 | 400.00 | sodium benzoate (0.01); |
| 25.00 | 200.00 | sodium benzoate (0.11); |
| 25.00 | 100.00 | formic + acetic + propionic (0.09); calcium formate (7.41); sodium benzoate (0.3); |
| 25.00 | 50.00 | formic + acetic + propionic (0.37); calcium formate (3.87); sodium benzoate (1.95); |
| 25.00 | 40.00 | formic + acetic + propionic + benzoic (0); |
| 25.00 | 25.00 | formic + acetic + propionic (0.43); sodium benzoate (5.93); |
| 25.00 | 20.00 | formic + acetic + propionic + benzoic (9.98); |
| 25.00 | 12.50 | formic + acetic + propionic (2.47); |
| 25.00 | 10.00 | formic + acetic + propionic + benzoic (13.82); |
| 25.00 | 6.25 | formic + acetic + propionic (10.3); sodium benzoate (24.65); |
| 25.00 | 5.00 | formic + acetic + propionic + benzoic (3.42); |
| 25.00 | 2.50 | formic + acetic + propionic + benzoic (15.29); |
| 25.00 | 1.56 | formic (21.82); propionic (4.06); |
| 12.50 | 200.00 | sodium benzoate (0.39); |
| 12.50 | 100.00 | formic + acetic + propionic (0.08); calcium formate (12.39); formic (2.39); |
| 12.50 | 50.00 | formic + acetic + propionic (0.33); calcium formate (4.4); formic (1.36); sodium benzoate (1.8); |
| 12.50 | 40.00 | formic + acetic + propionic + benzoic (0.04); |
| 12.50 | 25.00 | acetic (21.11); formic + acetic + propionic (0.19); sodium benzoate (2.62); |
| 12.50 | 20.00 | benzoic (0.86); formic + acetic + propionic + benzoic (10.49); |
| 12.50 | 12.50 | sodium benzoate (1.37); |
| 12.50 | 10.00 | formic + acetic + propionic + benzoic (9.25); |
| 12.50 | 6.25 | acetic (21.49); formic + acetic + propionic (5.2);formic (8.47); sodium benzoate (24.85); |
| 12.50 | 5.00 | formic + acetic + propionic + benzoic (7.61); |
| 12.50 | 3.13 | acetic (22.13); |
| 12.50 | 2.50 | formic + acetic + propionic + benzoic (0.93); |
| 12.50 | 1.56 | acetic (8.26); formic + acetic + propionic (14.9); |
| 12.50 | 1.25 | formic + acetic + propionic + benzoic (14.07); |
| 12.50 | 0.31 | benzoic (1.9); |
| 6.25 | 400.00 | calcium formate (3.13); sodium benzoate (0.09); |
| 6.25 | 100.00 | formic + acetic + propionic (0.3); formic (2); propionic (1.51); |
| 6.25 | 50.00 | formic + acetic + propionic (0.38); calcium formate (1.84); formic (1.71); |
| 6.25 | 25.00 | formic + acetic + propionic (0.31); propionic (7.65); |
| 6.25 | 20.00 | benzoic (0.85); formic + acetic + propionic + benzoic (11.29); |
| 6.25 | 12.50 | formic (7.96); propionic (1.97); |
| 6.25 | 10.00 | formic + acetic + propionic + benzoic (2.12); |
| 6.25 | 6.25 | formic + acetic + propionic (5.73); sodium benzoate (10.41); propionic (4.6); |
| 6.25 | 5.00 | benzoic (2.59); formic + acetic + propionic + benzoic (3.84); |
| 6.25 | 3.13 | formic + acetic + propionic (14.29); propionic (37.32); |
| 6.25 | 2.50 | benzoic (2.39); formic + acetic + propionic + benzoic (18.9); |
| 6.25 | 1.56 | acetic (34.4); formic + acetic + propionic (5.11 formic (38.2); |
| 6.25 | 1.25 | ); formic + acetic + propionic + benzoic (14.76); |
| 3.13 | 100.00 | calcium formate (10.91); formic (6.74); propionic (2.02); |
| 3.13 | 80.00 | formic + acetic + propionic + benzoic (0.02); |
| 3.13 | 50.00 | calcium formate (6.5); formic (4.08); |
| 3.13 | 40.00 | formic + acetic + propionic + benzoic (0.05); |
| 3.13 | 25.00 | propionic (6.88); |
| 3.13 | 20.00 | benzoic (1.02); formic + acetic + propionic + benzoic (10.57); |
| 3.13 | 12.50 | formic (30.05); |
| 3.13 | 10.00 | formic + acetic + propionic + benzoic (9.38); |
| 3.13 | 6.25 | formic + acetic + propionic (4.35); sodium benzoate (4.59); |
| 3.13 | 5.00 | benzoic (2.11); formic + acetic + propionic + benzoic (14.08); |
| 3.13 | 3.13 | propionic (20.02); |
| 3.13 | 2.50 | formic + acetic + propionic + benzoic (35.36); |
| 3.13 | 1.25 | formic + acetic + propionic + benzoic (26.7); |
| 1.56 | 100.00 | acetic (2.03); calcium formate (10.7); |
| 1.56 | 50.00 | acetic (1.98); |
| 1.56 | 40.00 | formic + acetic + propionic + benzoic (0.11); |
| 1.56 | 25.00 | acetic (11.57); |
| 1.56 | 20.00 | benzoic (1.02); formic + acetic + propionic + benzoic (11.17); |
| 1.56 | 12.50 | acetic (10.44); |
| 1.56 | 10.00 | formic + acetic + propionic + benzoic (0.24); |
| 1.56 | 6.25 | formic + acetic + propionic (24.84); sodium benzoate (6.87); |
| 1.56 | 5.00 | formic + acetic + propionic + benzoic (21.6); |
| 1.56 | 3.13 | acetic (11.34); |
| 1.56 | 2.50 | formic + acetic + propionic + benzoic (37.18); |
| 1.56 | 1.56 | acetic (21.5); |
| 1.56 | 1.25 | formic + acetic + propionic + benzoic (24.5); |
| 1.56 | 0.31 | benzoic (12.23); |
| 0.78 | 100.00 | formic + acetic + propionic (0.25); calcium formate (20.76); formic (3.62); |
| 0.78 | 50.00 | formic + acetic + propionic (0.21); formic (2.75); |
| 0.78 | 25.00 | acetic (17.98); formic + acetic + propionic (0.21); |
| 0.78 | 20.00 | formic + acetic + propionic + benzoic (10.76); |
| 0.78 | 12.50 | formic (10.57); |
| 0.78 | 6.25 | formic + acetic + propionic (19.15); propionic (26.92); |
| 0.78 | 5.00 | formic + acetic + propionic + benzoic (5.56); |
| 0.78 | 3.13 | formic + acetic + propionic (14.4); |
| 0.78 | 2.50 | formic + acetic + propionic + benzoic (20.38); |
| 0.78 | 1.25 | formic + acetic + propionic + benzoic (6.98); |
| 0.39 | 200.00 | calcium formate (6.25); |
| 0.39 | 100.00 | acetic (2.76); calcium formate (0.3); formic (1.5); sodium benzoate (0.15); propionic (0.98); |
| 0.39 | 50.00 | acetic (2.18); formic (0.85); propionic (0.41); |
| 0.39 | 25.00 | acetic (47.27); |
| 0.39 | 20.00 | formic + acetic + propionic + benzoic (9.47); |
| 0.39 | 12.50 | acetic (11.32); formic + acetic + propionic (0.4); formic (5.51); |
| 0.39 | 10.00 | formic + acetic + propionic + benzoic (0.79); |
| 0.39 | 6.25 | acetic (4.83); formic + acetic + propionic (24.3); formic (8.61); |
| 0.39 | 3.13 | acetic (72.31); propionic (0.31); |
| 0.39 | 2.50 | formic + acetic + propionic + benzoic (13.46); |
| 0.39 | 1.56 | acetic (22.27); formic (13.08); |
| 0.20 | 400.00 | calcium formate (0.28); sodium benzoate (0.1); |
| 0.20 | 200.00 | sodium benzoate (0.45); |
| 0.20 | 100.00 | calcium formate (15.66); formic (3.09); sodium benzoate (0.41); |
| 0.20 | 80.00 | formic + acetic + propionic + benzoic (0.06); |
| 0.20 | 50.00 | formic + acetic + propionic (0.23); calcium formate (5.06); formic (2.04); sodium benzoate (0.4); |
| 0.20 | 40.00 | formic + acetic + propionic + benzoic (0.49); |
| 0.20 | 25.00 | acetic (17.67); formic + acetic + propionic (0.25); |
| 0.20 | 20.00 | formic + acetic + propionic + benzoic (12.1); |
| 0.20 | 10.00 | formic + acetic + propionic + benzoic (16.2); |
| 0.20 | 6.25 | formic + acetic + propionic (29.94); |
| 0.20 | 5.00 | formic + acetic + propionic + benzoic (8.72); |
| 0.20 | 3.13 | formic + acetic + propionic (6.34); |
| 0.20 | 2.50 | formic + acetic + propionic + benzoic (31.14); |
| 0.20 | 1.56 | formic + acetic + propionic (12.04); |
| 0.20 | 1.25 | formic + acetic + propionic + benzoic (4.52); |

### Example 2: Modulating Microbial Growth in Complex Matrices

To verify the applicability of a composition comprising one or more organic acid(s) (or salt(s) thereof) and one or more hypophosphorous acid(s) (or salt(s) thereof), compositions according to the invention were used to modulate microbial growth in a nutritional composition. As exemplary nutritional composition a typical pig feed was used (e.g. Masching et al. 2016. Toxins 8:(3):84; Schwartz-Zimmermann et al. 2018. World Mycotoxin Journal, DOI 10.3920/WMJ2017.2265). More specifically, the nutritional composition was mixed with Mueller-Hinton broth, as well as with a composition comprising organic acid and hypophosphite to a concentration of either 5 mM or 12.5 mM of organic acid and 2 mM of sodium hypophosphite or magnesium hypophosphite or manganese hypophosphite or potassium hypophoshite. Then, a mid-to-late exponential culture of E. coli was added to a final inoculant density of 5*105 cfu/mL. To create anaerobic conditions an enzymatic system suitable for removing oxygen from a media (e.g. Oxyrase® by Sigma-Aldrich; Spangler et al. 1993. Journal of Clinical Microbiology 31 (2): 460-462) was added to the vessels and the mixtures were flushed with nitrogen and kept anaerobic and at 37° C. for the course of the experiment. Regularly throughout the incubation, 5 mL samples were drawn from the vessels and filtered (pore size 40 μm) to exclude large particles from the nutritional composition but without removing microbial cells. The filtered samples were used to create a two-fold serial dilution in Dulbecco's phosphate buffered saline (8 g/L NaCl, 0.2 g/L KCl, 0.2 g/L KH₂PO₄, 1.15 g/L Na₂HPO₄, pH 7.4) and 1 mL of those dilutions were used for plating on tryptone bile x-glucuronide agar (Merck) for colony counting. The prepared agar plates were incubated and E. coli colonies were counted. If necessary, dilution steps were added or reduced to allow convenient colony counting as known by a person having skill in the art.

It was found that compositions comprising one or more organic acid(s) (or salt(s) thereof) and one or more hypophosphorous acid(s) (or salt(s) thereof) were not only capable of modulating microbial growth in vitro as shown in Example 1, but also in a practical environment, i.e. in a complex matrix such as in a nutritional compositions (e.g. food or feed). Examplarily, compositions comprising 2 mM sodium hypophosphite and 5 mM benzoic acid; or comprising 2 mM sodium hypophosphite and 12.5 mM formic acid were found to lead to 97.69%; or 99.65% inhibition of E. coli growth, respectively. Notably, with regard to typical probiotic bacteria (L. reuteri, E. faecium, B. subtilis), growth inhibition is considerably lower than of potentially pathogenic bacteria (e.g. E. coli, S. enterica, S. sonnei, C. perfringens)—as also shown in Example 1.

1. Composition for modulating microbial growth, characterized in that the composition comprises at least one organic acid and/or at least one salt thereof; and hypophosphorous acid and/or at least one salt thereof.

2. The composition according to claim 1, wherein the hypophosphorous acid and/or the at least one salt thereof is/are selected from the group consisting of sodium hypophosphite, magnesium hypophosphite, manganese hypophosphite, potassium hypophosphite, aluminum hypophosphite, calcium hypophosphite, ammonium hypophosphite, ferric hypophosphite, preferably sodium hypophosphite, magnesium hypophosphite, manganese hypophosphite or potassium hypophosphite, more preferably sodium hypophosphite.

3. The composition according to claim 1, wherein the at least one organic acid and/or the at least one salt thereof is/are selected from the group consisting of short monocarboxylic acids having between 1 and 6 carbon atom(s), saturated dicarboxylic acids, unsaturated dicarboxylic acids, unsaturated carboxylic acids, saturated carboxylic acids, hydroxycarboxylic acids, aromatic carboxylic acids, and keto carboxylic acids, and/or at least one salt thereof.

4. The composition according to claim 3, wherein the at least one organic acid and/or the at least one salt thereof is selected from the group consisting of formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, gluconic acid, suberic acid, malonic acid, tannic acid, caffeic acid, ellagic acid, perillic acid, gallic acid, or at least one salt thereof (e.g. ammonium formate, potassium diformate, sodium diacetate, calcium acetate, ammonium propionate, sodium propionate, calcium propionate, calcium lactate, potassium sorbate, sodium formate, calcium formate, sodium butyrate, sodium benzoate, sodium sorbate, potassium citrate, sodium citrate, calcium citrate); preferably from formic acid, acetic acid, propionic acid, butyric acid, isobutyric acid, 3-methylbutyric acid, 2-metylbutyric acid, 2-ethylbutyric acid, valeric acid, hexanoic acid, adipic acid, succinic acid, fumaric acid, sorbic acid, oleic acid, stearic acid, octanoic (caprylic) acid, decanoic (capric) acid, dodecanoic (lauric) acid, lactic acid, malic acid, citric acid, tartaric acid, benzoic acid, cinnamic acid, pyruvic acid, or at least one salt thereof; more preferably from acetic acid, benzoic acid, butyric acid, citric acid, formic acid, fumaric acid, lactic acid, octanoic acid, propionic acid, pyruvic acid, sorbic acid, succinic acid, valeric acid, or at least one salt thereof.

5. The composition according to claim 1, wherein the molar ratio of the at least one organic acid and/or the at least one salt thereof to the hypophosphorous acid and/or at least one salt thereof is from 0.1:200 to 500:0.1, preferably from 0.16:100 to 400:0.2 (e.g. from 0.31:100 to 100:0.2).

6. The composition according to claim 1, wherein the composition comprises at least three organic acids, especially formic acid, acetic acid, propionic acid, and a salt of hypophosphorous acid selected from the group consisting of sodium hypophosphite, manganese hypophosphite, magnesium hypophosphite and potassium hypophosphite, preferably sodium hypophosphite or manganese hypophosphite, more preferably sodium hypophosphite.

7. The composition according to claim 6, wherein the composition further comprises benzoic acid.

8. Additive, such as foodstuff additive; fodder additive; feed additive; silage additive, comprising a composition for modulating microbial growth according to claim 1.

9. Method for modulating microbial growth, the method comprising the steps of
a) providing a composition for modulating microbial growth according to claim 1;
b) applying a) to a foodstuff; fodder; feed; foodstuff additive; fodder additive; feed additive; silage additive; wet distillers grain; dried distillers grain with solubles; nutritional supplement; prebiotic; probiotic; intermediate/s thereof; and/or mixture/s thereof.

10. The method according to claim 9, wherein the microbial growth is the growth of at least one microbe selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum, preferably from Enterobacter, Escherichia, Salmonella, Shigella, Yersinia, Vibrio, Aeromonas, Clostridium, Staphylococcus, Brachyspira, Streptococcus, more preferably from Escherichia, Salmonella, Shigella, Clostridium, most preferably the microbial growth is the growth of at least one microbe selected from the species of Escherichia coli, Salmonella enterica, Shigella sonnei, Clostridium perfringens.

11. A composition for use as a medicament and/or in treatment, amelioration, prophylaxis and/or diagnostics of a disease, such as in veterinary medicine, characterized in that the composition comprises hypophosphorous acid and/or at least one salt thereof, and optionally at least one organic acid and/or at least one salt thereof.

12. A composition for use in the treatment, amelioration and/or prophylaxis of dysbiosis, characterized in that the composition comprises hypophosphorous acid and/or at least one salt thereof, and optionally at least one organic acid and/or at least one salt thereof.

13. The composition for use in the treatment, amelioration and/or prophylaxis of dysbiosis according to claim 12, wherein the dysbiosis is caused by atypical concentration of at least one microorganism selected from the genera of Buttiauxella, Citrobacter, Cronobacter, Enterobacter, Escherichia, Edwardsiella, Klebsiella, Phytobacter, Plesiomonas, Pseudoescherichia, Raoultella, Salmonella, Shigella, Proteus, Yersinia, Vibrio, Aeromonas, Clostridium, Pseudomonas, Staphylococcus, Pasteurella, Brachyspira, Campylobacter, Listeria, Streptococcus, Haemophilus, Brucella, Enterococcus, Moritella, Tenacibaculum, Lactobacillus, Bifidobacterium, Saccharomyces, Bacillus, Pediococcus, Enterococcus, Streptococcus, Propionibacterium, Pseudomonas, Citrobacter, preferably from Enterobacter, Escherichia, Salmonella, Shigella, Yersinia, Vibrio, Aeromonas, Clostridium, Staphylococcus, Brachyspira, Streptococcus, Lactobacillus, Bifidobacterium, Pediococcus, Enterococcus, more preferably from Escherichia, Salmonella, Shigella, Clostridium, Bacillus, Lactobacillus, Enterococcus, most preferably the at least one microorganism is selected from the species of Escherichia coli, Salmonella enterica, Shigella sonnei, Clostridium perfringens, Lactobacillus reuteri, Enterococcus faecium, Bacillus subtilis.

14. A composition for use in the treatment, amelioration and/or prophylaxis of post-weaning diarrhea, characterized in that the composition comprises
hypophosphorous acid and/or at least one salt thereof, and optionally
at least one organic acid and/or at least one salt thereof.

15. Use of hypophosphorous acid and/or at least one salt thereof; and/or a composition according to claim 1 for modulating microbial growth.

16. The use according to claim 15, wherein the microbial growth of at least one microbe selected from Escherichia coli, Salmonella enterica, Shigella sonnei and Clostridium perfringens is reduced.
